(19) United States
(12) Patent Application Publication
(10) Pub. No.: US 2022/0249399 A1
(43) Pub. Date: Aug. 11, 2022

(54) COMPOSITION COMPRISING AN ANTI-OXIDANT TO PRESERVE CORNEAL TISSUE

(71) Applicant: University of lowa Research Foundation, lowa City, IA (US)

(72) Inventors: Aliasger K. Salem, Coralville, IA (US); Youssef Wahib Naguib lbrahim, lowa City, IA (US); Somaya Ali Mohammed Elsaid Abdelrahman, Redwood City, CA (US); Jessica M. Skeie, Ronald, WA (US); Benjamin T. Aldrich, Waukee, IA (US); Gregory Schmidt, Kalona, IA (US); Cynthia R. Reed, Solon, IA (US); Mark A. Greiner, lowa City, IA (US); Darryl Y. Nishimura, Coralville, IA (US); Sanjib Saha, lowa City, IA (US)

(21) Appl. No.: 17/436,042

(22) PCT Filed: Mar. 4, 2020
(86) PCT No.: PCT/US2020/020985

§ 371 Date: Sep. 2, 2021

Related U.S. Application Data
(60) Provisional application No. 62/813,559, filed on Mar. 4, 2019.

(51) Int. Cl. A61K 31/09; A61K 38/38; A61K 47/40; A61K 31/66; A61K 31/665; A01N 1/02

(52) U.S. Cl. CPC A61K 31/09; A61K 38/385; A01N 1/0226; A61K 31/66; A61K 31/665; A61K 47/40

(57) ABSTRACT

A composition comprising an amount of an anti-oxidant comprising one or more of ubiquinol, MitoQ, vitamin E, vitamin C, ascorbate-2-phosphate, idebenone, pyrroloquinoline quinone (PQQ), N-acetyl-L-cysteine (NAC), palmitate, reduced glutathione, or a C14-C18 saturated fatty acid effective to preserve, e.g., corneal tissue, and methods of using the composition, are provided.

### CROSS-REFERENCE TO RELATED APPLICATIONS

This application claims the benefit of the filing date of U.S. application No. 62/813,559, filed on Mar. 4, 2019, the disclosure of which is incorporated by reference herein.

### BACKGROUND

The corneal endothelium—the inner layer of the cornea which is comprised of corneal endothelial cells—is critical for deturgescence of the corneal stroma through its barrier and pump functions. Although central endothelial cell density (ECD) decreases with age, it decreases at a higher rate with corneal and ocular pathological conditions, such as Fuchs endothelial corneal dystrophy, diabetes mellitus, or following cataract surgery, glaucoma surgery, or cornea transplant surgery (keratoplasty). Approximately 30% of the corneal endothelial cells comprising the inner layer of the cornea die within 6 months following cornea transplant surgery to replace the corneal endothelium; yet, the cause is not fully understood. Failure of the corneal endothelium to recover from corneal endothelial cell damage is due to its limited ability to divide. If central ECD falls below a critical level with an insufficient number of endothelial cells and their associated sodium-potassium adenosine triphosphatase (ATP) pump sites to dehydrate the stroma, the cornea swells, vision decreases, and keratoplasty using donor corneal endothelial cells (CECs) is then indicated.

During life, the human cornea is increasingly susceptible to damage from reactive oxygen species (ROS) due to its elevated exposure to ultraviolet light (UV), exposure to dioxygen, and increased energy demands where ROS are an unavoidable byproduct. Elevated levels of ROS lead to protein, lipid, and DNA modifications and damage, eventually inducing cell death. Corneal dysfunction and endothelial cell death in Fuchs endothelial cell dystrophy—the most common disease of the corneal endothelial cell layer that affects 4% of the U.S. population and is the leading indication for cornea transplant surgery—is attributed to elevated ROS in the setting of genetic susceptibility. Also, in an animal model it has been shown that CECs have elevated levels of ROS following penetrating keratoplasty. Thus, it has been established that CECs show an increase in ROS when cells are stressed or damaged in vivo. Of note, currently there is no commercially available medical therapy such as a topically applied antioxidant drop to reduce oxidative damage from Fuchs endothelial corneal dystrophy or after cornea transplant surgery.

Because medical therapies are lacking to preserve the health of corneal endothelial cells, it is important to develop such medical therapies to prevent corneal transplant surgeries, and equally important to mitigate endothelial cell loss that occurs with corneal transplant surgery. Two important prospective studies of cornea transplant surgery have investigated various perioperative aspects in order to better understand cell loss with cornea transplant surgery: the Cornea Donor Study and the Cornea Preservation Time Study.

The Cornea Donor Study and its ancillary study, the Specular Microscopy Ancillary Study, which evaluated the effect of donor age on graft success and endothelial cell loss (ECL) following penetrating keratoplasty (PK), demonstrated the importance of ECL in estimating long-term graft survival. Five years after PK, graft success rates were similar with older and younger donor age, but ECL was greater with corneas from older donors compared with those from younger donors. This ECL difference at 5 years presaged a higher graft failure rate in the older donor age group by 10 years. In addition, ECD at 6 months, 1 year, and 5 years was associated at each time point with subsequent graft failure, irrespective of donor age.

The Cornea Preservation Time Study (CPTS) was designed to determine whether the success of Descemet stripping automated endothelial keratoplasty (DSAEK) performed for corneal conditions associated with endothelial failure is related to donor cornea preservation time (PT). With the Cornea Donor Study and the results of studies examining ECL following DSAEK in mind, the determination of ECL and its association with PT following DSAEK was considered an important outcome in designing the CPTS protocol, particularly since there have been few clinical studies assessing the effect of PT on ECL following keratoplasty with hypothermic (2° C.-8° C.) storage solutions. None of these previous studies examined the clinical performance of these solutions for the full 14 days approved by the US Food and Drug Administration. The CPTS was a multicenter, double-masked, randomized clinical trial. Forty US clinical sites with 70 surgeons participated, with donor corneas provided by 23 US eye banks. Individuals undergoing DSAEK for Fuchs dystrophy or pseudophakic/aphakic corneal edema were included.

In the Cornea Preservation Time Study's main outcome manuscript, Rossenwasser et al. (2018) determined that the 3-year success rate in eyes undergoing DSAEK was relatively high for all groups analyzed. However, the study was unable to conclude that the success rate with donor corneas preserved 8 to 14 days was similar to that of corneas preserved 7 days or less with respect to the prespecified noninferiority limit. Longer PT was associated with a lower success rate, with PT of 12-14 days decreased graft survival compared to PT≤11 days as follows: success rates of 96.5% (95% CI, 92.3%-98.4%) for PT of 4 days or less; 94.9% (95% CI, 92.5%-96.6%) for PT of 5 to 7 days; 93.8% (95% CI, 91.0%-95.8%) for PT of 8 to 11 days, and 89.3% (95% CI, 84.4%-92.7%) for PT of 12 to 14 days (P=0.01 [PT analyzed as categorical variable]).

Additionally, Lass et al. (2017) evaluated whether endothelial cell loss 3 years after successful DSAEK surgery was related to PT. The authors found that increasing preservation time is associated with increased endothelial cell loss, as follows: at 3 years, the mean (SD) ECD decreased from baseline by 37% (21%) in the 0-7d PT group and 40% (22%) in the 8-14d PT group to 1722 (626) cells/mm² and 1642 (631) cells/mm², respectively (mean difference, 73 cells/mm²; 95% CI, 8-138 cells/mm²; P=0.03). When analyzed as a continuous variable (days), longer PT was also associated with lower ECD (mean difference by days, 15 cells/mm²; 95% CI, 4-26 cells/mm²; P=0.006). Thus, the duration of time that CECs spend in hypothermic storage has a significant clinical impact on cornea transplant survival and endothelial cell health. Lass et al. (2019) also evaluated the associations of donor, recipient, and operative factors with ECD 3 years after DSAEK in the Cornea Preservation Time Study. The authors found that donor diabetes, lower screening ECD, a diagnosis of pseudophakic or aphakic corneal edema in the recipient, and operative complications were associated with lower ECD at 3 years after DSAEK surgery and may be associated with long-term graft success. Thus, the exposure of CECs to donor and recipient disease states prior to procurement and after transplantation have significant clinical impact on cornea transplant survival and endothelial cell health.

Findings from the CPTS indicate clearly that preservation time in hypothermic storage is clinically significant. Other organ and tissue hypothermic storage studies have shown that cold storage strategies to preserve tissue function by reducing metabolic strain paradoxically increases ROS and inflammation, especially when the organ/tissue is returned to body temperature.

### SUMMARY

The disclosure provides a corneal preservation composition comprising an effective amount of an anti-oxidant comprising one or more of ubiquinol, mitoquinone mesylate (MitoQ), idebenone, vitamin E, vitamin C (ascorbate), pyrroloquinoline quinone (PQQ), N-Acetyl-L-cysteine (NAC), palmitate, ascorbate-2-phosphate, reduced glutathione, or a C14-C18 fatty acid, or any combination thereof. In one embodiment, the amount is cytoprotective, decreases ROS, decreases corneal endothelial cell death, decreases apoptosis, decreases necrosis, increases mitochondrial function, increase mitochondrial or non-mitochondrial cellular respiration, allows for maintenance of ECD, or any combination thereof. In one embodiment, the fatty acid is a saturated C14-C18 fatty acid, e.g., comprises palmitic acid or BSA-palmitate. In one embodiment, the composition further comprises an amount of chondroitin sulfate or one or more omega 3 fatty acids. In one embodiment, the omega 3 fatty acid comprises docosahexaenoic acid (DHA), eicosapentaenoic acid (EPA) and/or alpha-linolenic acid. In one embodiment, the composition further comprises one or more carriers. In one embodiment, the carrier comprises cyclodextrin. In one embodiment, the carrier comprises polyethylene glycol (PEG), e.g., having molecular weights of about 1,000, 2,000, 2,500, 3,000, 3,500 or 4,000, PEG dodecyl ether (Brij L4®), PEG hexadecyl ether (Brij 58®), lipid-based solubilizers like Labrafil® and Labrafac®, pluronics, e.g., Pluronic F68 (Poloxamer 188), polysorbate 80 and 20 or lipid nanoparticles. In one embodiment, if the carrier is a surfactant, the surfactant ratio is about 2:1 to 1:10. In one embodiment, the carrier comprises PEG at about 0.1% to about 0.8% or about 0.3% to about 0.5%. In one embodiment, the anti-oxidant comprises solubilized ubiquinol. In one embodiment, the composition is formulated for topical eye drops. In one embodiment, the composition is formulated for injection. In one embodiment, the composition is a powder. In one embodiment, the composition is associated with a contact lens. In one embodiment, the composition is associated with a punctal plug. In one embodiment, the composition is associated with a wearable ocular ring. In one embodiment, the composition is a tablet, e.g., which may be placed in a corneal compatible medium. In one embodiment, the composition further comprises a full thickness cornea, e.g., which is stored at 2-40° C. for less than a day or up to 3, 5, 7, 10, 12, 14, 21 or 28 or more days. In one embodiment, the composition further comprises a partial thickness cornea. In one embodiment, the composition further comprises corneal endothelium. In one embodiment, the full or partial thickness cornea or corneal endothelium is human. In one embodiment, the anti-oxidant and the carrier form complexes. In one embodiment, the complexes are about 200 to about 400 nm in diameter. In one embodiment, the ubiquinol or idebenone in the composition is about 0.05 μM to about 100 μM, e.g., about 0.05 μM to about 5 μM or about 7 μM to about 15 μM or about 10 μM to about 30 μM or about 30 μM to about 50 μM. In one embodiment, the concentration of vitamin C or ascorbate-2-phosphate is about 0.1 μM to about 10 μM, about 0.1 μM about 0.4 μM or about 0.2 μM to about 0.3 μM. In one embodiment, the concentration of vitamin A is about 0.05 μM to about 10 μM, about 0.3 μM to about 0.7 μM about 0.4 μM to about 0.6 μM, or about 50 μM to about 1 mM. In one embodiment, the concentration of vitamin E is about 0.1 μM to about 10 μM, about 0.01 μM to about 0.04 μM or about 0.015 μM to about 0.03 μM. In one embodiment, the concentration of PQQ is about 0.1 μM to about 100 μM, e.g., about 1 μM to about 50 μM or about 5 μM to about 15 μM. In one embodiment, the concentration of NAC is about 0.1 mM to about 10 mM, e.g., about 0.1 mM to 50 mM or about 0.5 mM to about 15 mM. In one embodiment, the concentration of palmitate-BSA is about 0.1 μM to about 750 μM, e.g., about 10 μM to about 500 μM. In one embodiment, the concentration of reduced glutathione about 0.1 μM to about 10 μM, about 0.05 μM to about 0.4 μM or about 0.1 μM to about 0.3 μM. In one embodiment, the concentration of the complexes in the composition comprises about 0.1 μM to about 5 μM. In one embodiment, the concentration of the complexes in the composition comprises about 5 μM to about 50 μM. In one embodiment, the concentration of the complexes comprises about 50 μM to about 150 μM. In one embodiment, the composition further comprises a base medium and one or more of chondroitin sulfate, dextran, insulin, a buffer, non-essential amino acids, or sodium bicarbonate. Ratios of ubiquinol to cyclodextrin may be 1:10, 1:2 or 1:5.

Also provided is a method of making complexes of one or more anti-oxidants comprising ubiquinol, idebenone, MitoQ, vitamin A, vitamin C, ascorbate-2-phosphate, PQQ, NAC, palmitate, reduced glutathione, vitamin E, or a C14-C18 saturated fatty acid, and a carrier, comprising: combining an amount of the one or more anti-oxidants and an amount of a carrier and conditions so as to form complexes of about 100 nm to about 1000 nm, e.g., about 100 nm to about 500 nm, in diameter. In one embodiment, the molar ratio of the anti-oxidant to the carrier is from x:y, where x and y are independently any integer between 1 and 1000, e.g., 1:1 to 1:1000, 2:1 to 1:10 or 3:1 to 1:20. In one embodiment, the molar ratio of ubiquinol to cyclodextrin, e.g., hydroxypropyl beta-cyclodextrin or gamma-cyclodextrin is about 1:15, 1:10, 1:5, or 1:20.

In one embodiment, the composition is for ophthalmic use, e.g., a topical eye drop in humans with corneal diseases including but not limited to Fuchs endothelial cell dystrophy and diabetes mellitus, e.g., and in humans with prior corneal transplant surgery including but not limited to partial thickness cornea transplant techniques and full thickness cornea transplant techniques. In one embodiment, the composition is for tissue preservation, e.g., of any tissue including but not limited to whole corneas, partial corneas, endothelium, for instance, corneal endothelium, epithelium, for instance, corneal epithelium.

Further provided is a method of preserving a cornea, corneal tissue or corneal endothelium of a mammal, comprising: providing a cornea, corneal tissue or corneal endothelium of a mammal; and combining the cornea, corneal tissue or corneal endothelium and the composition described herein. In one embodiment, the mammal is a human.

In addition, a method of treating corneal tissue, e.g., corneal endothelium, corneal epithelium, corneal keratocytes, corneal stroma, or corneal nerves, conjunctival epithelium, conjunctival stroma, Tenon's capsule, trabecular meshwork, corneoscleral angle, lens epithelium, or lens in a mammal is provided. The method comprises administering to a mammal in need thereof an effective amount of the composition described herein. In one embodiment, the mammal is a human, e.g., an individual with an ocular disease such as diabetes or Fuchs endothelial cell dystrophy, or an individual that will undergo ocular surgery such as cataract surgery, cornea transplant surgery, corneal surgery, ocular surface surgery including pterygium excision and lesion biopsy, e.g., and intravitreal surgery, and vitreoretinal surgery. In one embodiment, the composition is injected into the anterior or posterior segment. The composition may be topically administered. The composition may be intraocularly administered.

The compositions disclosed herein may be delivered by any device, e.g., drug eluting intraocular devices, e.g., in the anterior or posterior segment, drug eluting ring devices placed on the eye surface, drug eluting devices implanted into the punctate of the lacrimal drainage system, or drug impregnated contact lens.

### BRIEF DESCRIPTION OF THE FIGURES

FIG. 1. High OCR of CEC supplemented with ubiquinol (red) compared to non-supplemented CEC (blue).

FIG. 2. A549 human endothelial lung cancer cells treatment with ubiquinol either as free drug, or in CD-complex, with or without antimycin A (AM). It can be seen that only ubiquinol in complex were able to significantly decrease ROS levels (outlined as dihydroethidium (DHE) fluorescence, p<0.05) following pre-treatment of cells with AM. Free ubiquinol was able to decrease the ROS levels in the cells below the normal levels, as did the complex, but the free drug failed to decrease ROS levels after AM treatment.

FIG. 3. Gamma-cyclodextrin-ubiquinol compositions (left) show high water dispersion compared to free ubiquinol.

FIG. 4. Mitochondrial respiration of corneal endothelial cells exposed to palmitate-BSA (red) or BSA alone (blue) is shown on left. Effects of exposure to palmitate-BSA and BSA on the corneal endothelium cell apoptosis and necrosis is shown on right.

FIGS. 5A-B. OCR results from sod2 null mice.

FIG. 6. Mitochondrial respiration of corneal endothelial cells exposed to enzyme CoQ10 (red) or BSA alone (blue) is shown on left. Effects of exposure to enzyme CoQ10 is shown on right.

FIG. 7. Seahorse results of two examples of immortalized human corneal endothelial cells treated with cyclodextrin-CoQ10.

FIG. 8. Mitochondrial ROS in relative fluorescence units (RFUs). Higher RFUs indicate higher levels of ROS.

FIG. 9. Complexes of different cyclodextrins.

FIG. 10. Left shows 50 mg of kneaded complex added to 10 mL H₂O and shaken for 2 hours. Right shows 5 mg CoQ10 added to 10 mL H₂O and shaken for 2 hours.

FIG. 11. Left shows 50 mg of kneaded complex added to 10 mL H₂O and shaken for 24 hours. Right shows 5 mg CoQ10 added to 10 mL H₂O and shaken for 24 hours.

FIG. 12. A) Differential scanning calorimetry. B) X-ray diffraction.

FIG. 13. Scanning electron microscopy.

FIG. 14. Samples for ROS assay.

FIG. 15. ROS assay results.

FIG. 16. Fluorescence assay results.

FIG. 17. HPLC analysis. Agilent 1100 series HPLC station with a Waters RP-C18 4.6×150 mm column, pore size 5 μm, set at room temperature. Mobile phase: Acetonitrile:THF:Water 60:35:5. Flow rate: 1 ml/min. Wavelength: 290 nm for ubiquinol, and 280 nm for coenzyme Q10 (ubiquinone) and coenzyme Q9. Injection volume: 50 μl.

FIG. 18. Amount of total CoQ10, ubiquinol and oxidized CoQ10 in complexes and not in complexes.

FIGS. 19A-19B. Seahorse assay with MitoQ. A) Oxygen consumption rates (pmol/min/cell; vertical axis) representing the ATP linked respiration of primary cultures of corneal endothelial cells treated with different concentrations of MitoQ (horizontal axis). Control and 10 μM treatments were significantly different (P<0.001) N=3. B) Oxygen consumption rates (pmol/min/cell; vertical axis) representing the spare respiratory capacity of primary cultures of corneal endothelial cells treated with different concentrations of MitoQ (horizontal axis). Control and 10 μM treatments were significantly different (P<0.001) N=3.

FIG. 20. Mitochondrial respiration assay using Seahorse XFe24 extracellular flux analyzer of human donor corneal endothelial cells following 5 days of storage in Optisol GS supplemented with 10 μM ubiquinol (red) compared to non-supplemented CECs (blue). Right panel: top and bottom figures show % necrotic and % apoptotic cells, respectively, of CEC following 5 days of storage in Optisol GS supplemented with 10 μM ubiquinol compared to non-supplemented CECs.

FIG. 21. Mitochondrial respiration assay using Seahorse XFe24 extracellular flux analyzer of human donor corneal endothelial cells following 5 days of storage in Optisol GS supplemented with 1 mM ascorbate 2-phosphate (red) compared to non-supplemented CECs (blue).

FIG. 22. Mitochondrial respiration assay using Seahorse XFe24 extracellular flux analyzer of human donor corneal endothelial cells following 5 days of storage in Optisol GS supplemented with 100 μM palmitate-BSA (red) compared to non-supplemented CECs (blue). Right panel: top and bottom figures show % necrotic and % apoptotic cells, respectively, of CEC following 5 days of storage in Optisol GS supplemented with palmitate-BSA compared to non-supplemented CECs.

FIGS. 23A-23B. Mitochondrial respiration assay using Seahorse XF24 extracellular flux analyzer of cortical synaptosomes isolated from Sod+/+ and Sod−/− mice.

FIG. 24. Mitochondrial ROS in relative fluorescence units (RFUs) of human immortalized corneal endothelial cells exposed to ubiquinol-γ-cyclodextrin complex (CyCoQ10) equivalent to ubiquinol concentration of 1 or 100 μM. Higher RFUs indicate higher levels of ROS. The lower concentration of complex significantly reduced levels of ROS (P<0.001), while the higher concentration did not (P=0.37).

FIG. 25. Left shows 50 mg of kneaded complex added to 10 mL H₂O and shaken for 2 hours. Right shows 5 mg CoQ10 added to 10 mL H₂O and shaken for 2 hours.

FIG. 26. Left shows 50 mg of kneaded complex added to 10 mL H₂O and shaken for 24 hours. Right shows 5 mg CoQ10 added to 10 mL H₂O and shaken for 24 hours.

FIGS. 27A-27B. A) Differential scanning calorimetry (DSC). B) X-ray diffraction (XRD).

FIG. 28. Scanning electron microscopy (SEM).

FIG. 29. A549 human epithelial lung cancer cells treatment with ubiquinol either as free drug, or in CD-complex, with or without antimycin A (AM, ROS inducer).

FIGS. 30A-30C. Flow cytometric histograms of A549 cells. The cells are either untreated (untreated no AM) or with 5 mM AM (untreated) (A), treated with ubiquinol/γ-cyclodextrin complex 1:10 equivalent to 100 μM ubiquinol (complex no AM) or with ubiquinol/γ-cyclodextrin complex 1:10 equivalent to 100 μM ubiquinol and 5 mM AM (complex) (C), and treated with 100 μM Ubiquinol (coenzyme Q10 no AM) or with 100 μM ubiquinol and 5 mM AM (coenzyme Q10) (B).

FIGS. 31A-31B. A) Results of the ROS assay using A549 cells stained with dihydroethidium (DHE) represented as the geometric mean of DHE fluorescence. It can be seen that only ubiquinol in complex with γ-cyclodextrin (1:10 molar ratio) were able to significantly decrease ROS levels (p<0.05) following pre-treatment of cells with AM. Free ubiquinol was able to decrease the ROS levels in the cells below the normal levels, as did the complex, without ROS induction by AM, but the free drug failed to decrease ROS levels after AM treatment. B) An increase in the concentration of ubiquinol in γ-cyclodextrin complex will result in a significant increase of ROS inhibition. An increase in the free ubiquinol concentration does not result in any significant change in ROS inhibition.

FIG. 32. HPLC chromatogram and HPLC conditions for the analysis of Ubiquinol, and ubiquinone (coenzyme Q10).

FIGS. 33A-33C. Amount of total CoQ10 (A), ubiquinol (B) and oxidized CoQ10 (C) taken up into A549 cells after incubation of either free ubiquinol or ubiquinol in γ-cyclodextrin (1:10 molar ratio) with the cells for 1 or 3 hours at 37° C. It was found that the amount Ubiquinol, oxidized Ubiquinol, and totals coenzyme Q10 taken up into cells following the treatment with the complex was significantly higher than that with free ubiquinol (p<0.05, n=4-6).

FIGS. 34A-34B. A) Oxygen consumption rates (pmol/min/cell; Y-axis) representing the ATP linked respiration of primary cultures of corneal endothelial cells treated with different concentrations of MitoQ (X-axis). Control and 10 μM treatments were significantly different (P<0.001, n=3). B) Oxygen consumption rates (pmol/min/cell; Y-axis) representing the spare respiratory capacity of primary cultures of corneal endothelial cells treated with different concentrations of MitoQ (X-axis). Control and 10 μM treatments were significantly different (P<0.001, n=3).

FIG. 35. Mean oxygen partial pressure (pO2) over time in Krolman chambers (N=3) with and without donor tissue. Oxygen was measured using a Fibox 4 oxygen sensor (PreSens Precision Sensing GmbH, Regensburg, Germany) and chambers remained sealed during measurements. During the full duration of storage, donor corneas are exposed to elevated oxygen concentrations (>69 mm Hg pO₂) that far exceed oxygen concentrations found beneath the endothelium in the anterior chamber of healthy patients (8-21 mm Hg pO₂). Error bars represent SEM. Differences between measurements at each time point statistical significance shown by *P<0.05 and **P<0.001. Inset details differences found during first day.

FIG. 36. Mitochondrial respiration assay using Seahorse XFe24 extracellular flux analyzer of human donor corneal endothelial cells following 5 days of storage in Optisol GS supplemented with 10 μM ubiquinol (red; n=13) compared to non-supplemented CECs (blue; n=13). Statistically significant changes included: spare respiratory capacity increased 174% (p=0.001), maximal respiration increased 93% (p=0.003), and proton leak increased 80% (p=0.047) compared to controls. Right panel: top and bottom figures show % necrotic and % apoptotic cells, respectively, of CEC following 5 days of storage in Optisol GS supplemented with 10 μM ubiquinol compared to non-supplemented CECs.

FIG. 37. Mitochondrial respiration assay using Seahorse XFe24 extracellular flux analyzer of human donor corneal endothelial cells following 5 days of storage in Optisol GS supplemented with 1 mM ascorbate 2-phosphate (red; n=5) compared to non-supplemented CECs (blue; n=5).

FIG. 38. Mitochondrial respiration assay using Seahorse XFe24 extracellular flux analyzer of human donor corneal endothelial cells following 5 days of storage in Optisol GS supplemented with 100 μM palmitate-BSA (red; n=7) compared to non-supplemented CECs (blue; n=7). Right panel: top and bottom figures show % necrotic and % apoptotic cells, respectively, of CEC following 5 days of storage in Optisol GS supplemented with palmitate-BSA compared to non-supplemented CECs. Cells treated with palmitate-BSA had a 90% increase in necrosis (p=0.024) and 200% increase in apoptosis (p=0.028).

FIG. 39. Mitochondrial respiration assay using Seahorse XFe24 extracellular flux analyzer of primary CECs. Left: Oxygen consumption rates (pmol/min/cell; Y-axis) representing the ATP linked respiration of primary cultures of CECs treated with different concentrations of MitoQ (X-axis). Control and 10 μM treatments were significantly different (P<0.001, n=3). Right: Oxygen consumption rates (pmol/min/cell; Y-axis) representing the spare respiratory capacity of primary cultures of CECs treated with different concentrations of MitoQ (X-axis). Control and 10 μM treatments were significantly different (P<0.001, n=3).

FIG. 40. Mitochondrial respiration assay using Seahorse XFe24 extracellular flux analyzer of immortalized human corneal endothelial cells following 25 days of growth in 5.5-13.0 mM glucose and treated with 8.3 μM PQQ (n=18). Maximal respiration and spare respiratory capacity are lowered in HCECs (P<0.0001) due to hyperglycemic growing conditions. The supplementation of the hyperglycemic medium with PQQ mitigates this effect (P<0.0001).

FIG. 41. Mitochondrial respiration assay using Seahorse XFe24 extracellular flux analyzer of immortalized human corneal endothelial cells following 25 days of growth in 5.5-13.0 mM glucose and treated with 1.0 mM NAC (n=18). Maximal respiration and spare respiratory capacity are lowered in HCECs (P<0.0001) due to hyperglycemic growing conditions. The supplementation of the hyperglycemic medium with NAC mitigates this effect (P<0.0001).

FIG. 42. Left: 50 mg of kneaded ubiquinol complexed with γ-cyclodextrin at a molar ratio of 1:10 (equivalent to 3.125 mg ubiquinol) added to 10 mL H₂O and shaken for 2 hours. Right: 5 mg ubiquinol alone added to 10 mL H₂O and shaken for 2 hours.

FIG. 43. Left: Differential scanning calorimetry (DSC), right: X-ray diffraction (XRD) of ubiquinol, γ-CD, ubiquinol/γ-CD physical mixture, and ubiquinol/γ-CD inclusion complex (molar ratio 1:10).

FIG. 44. Scanning electron microscopy (SEM) of ubiquinol, γ-CD, ubiquinol/γ-CD physical mixture, and ubiquinol/γ-CD inclusion complex (molar ratio 1:10). Top panel: low magnification, middle panel: intermediate magnification, bottom panel: high magnification.

FIG. 45. Stability of ubiquinol alone versus ubiquinol complexed with γ-cyclodextrin in Optisol GS. Stability is measured with regard to ubiquinol (the reduced form), ubiquinone (the oxidized form), and total coenzyme Q10 (coQ10).

FIG. 46. Flow cytometric histograms of A549 cells (top), and the bar graph figures (middle and bottom) representing the values obtained from the statistical analysis (geometric means) of the DHE fluorescence signals from histograms (values are means±SD).

FIG. 47. HPLC chromatogram and HPLC conditions for the analysis of ubiquinol and ubiquinone.

FIG. 48. Amount of total coQ10, ubiquinol and oxidized ubiquinol (ubiquinone) taken up into A549 cells after incubation of either free ubiquinol or ubiquinol complexed with γ-cyclodextrin (1:10 molar ratio) with the cells for 1 or 3 hours at 37° C. It was found that the amounts of ubiquinol, oxidized ubiquinol, and total coQ10 taken up into cells following the treatment with the complex were significantly higher than those with free ubiquinol (p<0.05, n=4-6).

FIG. 49. Flow cytometric histograms of human immortalized corneal endothelial cells (bottom), and the bar graph figure (top) representing the values obtained from the statistical analysis (geometric means) of the DHE fluorescence signals from histograms (values are means±SD).

FIG. 50. Left panel: coumarin/γ-cyclodextrin complex (1:10) prepared using the same method used for ubiquinol. Right panel: complexed coumarin shows much higher corneal penetrance compared to free coumarin. Fresh porcine corneas were fixed in Ussing diffusion cells (epithelial side facing the donor compartment). After 2h of treatment with either complexed coumarin or free coumarin, the corneas were removed from the diffusion cells, rinsed thoroughly in PBS, attached on a slide cover on an anti-fade mounting medium (ProLong Gold Antifade reagent), then imaged under confocal microscope. The complexed coumarin was able to penetrate the corneas and reached the endothelial side, while the free coumarin could not.

### DETAILED DESCRIPTION

The human corneal endothelium, made of a single layer of hexagonal corneal endothelial cells (CECs), keeps the cornea clear by pumping ions to counteract the passive leak of fluid into the stroma. Activity of these cells is energy dependent, requiring ATP produced via aerobic mitochondrial metabolism under normoxic conditions. If ionic pumping fails for any reason, fluid accumulates in the cornea, resulting in reduced corneal clarity and visual acuity. Mitochondrial health and function are vital for proper CEC function, and alterations in mitochondrial function appear to impact the health of transplanted and native corneal tissue. The cornea is susceptible to damage from reactive oxygen species (ROS) due to its elevated exposure to UV, exposure to dioxygen, and increased energy demands where ROS are an unavoidable byproduct. Elevated levels of ROS lead to protein, lipid, and DNA modifications and damage, eventually inducing cell death. Corneal dysfunction in Fuchs endothelial cell dystrophy, the most common corneal endotheliopathy, is attributed to elevated ROS in the setting of genetic susceptibility. Also, in an animal model it has been shown that CECs have elevated levels of ROS following penetrating keratoplasty. Thus, it has been established that CECs show an increase in ROS when cells are stressed or damaged.

Corneas preserved in conventional hypothermic storage media such as Optisol-GS (Bausch+Lomb, Rochester, N.Y.) have reduced graft survival with increasing preservation time (PT). Currently, donor cornea tissue can be stored per U.S. Food and Drug Administration guidelines up to 14 days at 4° C. in approved corneal storage media. Prospective investigations from the Cornea Preservation Time Study have shown, however, that PT of 12-14 days decreases graft survival and endothelial cell loss increases with PT 3 years after Descemet stripping automated endothelial keratoplasty (DSAEK). Other organ and tissue hypothermic storage studies have shown that cold storage strategies to preserve tissue function by reducing metabolic strain paradoxically increases ROS and inflammation, especially when the organ/tissue is returned to body temperature. Oxygen concentrations were measured using a Fibox 4 oxygen sensor (PreSens, Regensburg, Germany). It was observed that pO₂ remains approximately 4× higher over the entire period (14 days) compared to normal anterior chamber pO₂ levels (FIG. 35). The exposure to supraphysiologic oxygen concentrations over preservation times up to 14 days, followed by the return to physiologic concentrations in the anterior chamber, may represent a source of significant oxidative stress on CECs.

Partial thickness corneal transplant procedures involve the transplant of only the corneal endothelium, as in Descemet stripping automated endothelial keratoplasty (DSAEK) and Descemet membrane endothelial keratoplasty (DMEK), rather than replacing the full thickness cornea as in penetrating keratoplasty (PK). DSAEK and DMEK are indicated whenever the corneal dysfunction is limited to the endothelium, while other corneal tissues are not primarily affected. Unfortunately, endothelial cell density (ECD) post-transplant drops by 25-37% within 6 months after DSAEK and/or DMEK. While this cell loss had been believed to occur during surgery, recent research indicates that tissue preparation prior to surgery is significantly involved. Corneal endothelial cells (CEC) health and functionality require energy, obtained via mitochondrial ATP production. Stressful conditions that may lead to decreased ECD include insufficient mitochondrial respiration and high oxidative stress with elevated levels of reactive oxygen species (ROS), as well as in ocular disease and surgery states including diabetes mellitus, Fuchs endothelial cell dystrophy, cataract surgery, glaucoma surgery or cornea transplant surgery. Controlling the ROS levels while maintaining mitochondrial respiration at high capacity may decrease endothelial cell death before ocular surgery and improve the overall ECD post-operatively

Coenzyme Q10 is a lipophilic anti-oxidant that is present in almost all animal and human tissues as either the reduced form (ubiquinol) or the oxidized form (ubiquinone) (Onur et al., 2014). It is an essential coenzyme for several processes involving mitochondrial electron transport, and its presence is crucial in the production of ATP by oxidative phosphorylation. Only the reduced form (ubiquinol) is active, and the oxidized form has to be reduced in the body by the action of NADPH to become functional. Supplementation of coenzyme Q10 was found to be beneficial in several diseases, including atherosclerosis, Parkinson disease, and stroke, where also high levels of ROS are directly involved. The delivery of readily active form ubiquinol, while considered superior to coenzyme Q10, is hindered by the facts that it is highly unstable, and practically water insoluble.

### Exemplary Compositions and Methods of Use

Compositions described herein include, in one embodiment, one or more anti-oxidants useful in corneal preservation media or formulations including but not limited to solutions, e.g., topically applied drops for ophthalmic use, lyophilized formulations, injections, tablets and the like, useful in that regard. In one embodiment, the compositions also include one or more carriers, e.g., carriers that may enhance the solubilization of the one or more anti-oxidants. Exemplary anti-oxidants include but are not limited to ubiquinol, idebenone, MitoQ, vitamin E, vitamin C, ascorbate-2-phosphate, PQQ, NAC, palmitate, reduced glutathione, or a C14-C18 saturated fatty acid. In one embodiment, exemplary carriers include but are not limited to cyclodextrin, polyethylene glycol (PEG), PEG dodecyl ether (Brij L4®), PEG hexadecyl ether (Brij 58®), lipid-based solubilizers like Labrafil® and Labrafac®, pluronics, e.g. Pluronic F68 (Poloxamer 188), polysorbate 80 and 20 or lipid nanoparticles. In one embodiment, the carrier is a surfactant. Optional agents that may be included in the compositions include but are not limited to chondroitin sulfate, dextran, insulin, a buffer such as HEPES buffer, non-essential amino acids, or sodium bicarbonate.

The compositions may be added to or mixed with other cornea compatible media including but not limited to Optisol, Optisol GS, Life4C, Cornea Cold, or Eusol; irrigating solutions such as those use during cataract surgery, e.g., BSS-Plus; biologically compatible media or buffers, e.g., PBS, media 199, MEM, DMEM, or Earl's balanced salt solution; ophthalmic solutions for clinical use including but not limited to preserved artificial tears or non-preserved artificial tears or combinations thereof.

In one embodiment, the composition comprises one or more of ubiquinol, idebeone, MitoQ, vitamin E, vitamin C, ascorbate-2-phosphate, PQQ, NAC, palmitate, reduced glutathione, or a C14-C18 saturated fatty acid, and in one embodiment further includes a cyclodextrin, base medium, chondroitin sulfate, dextran, HEPES buffer, non-essential amino acids, sodium pyruvate and sodium bicarbonate, which composition is serum-free. In one embodiment, the ubiquinol or idebenone in the composition is about 0.05 μM to about 100 μM, e.g., 0.05 to about 5 μM or about 7 μM to about 15 μM. In one embodiment, the concentration of vitamin C or ascorbate-2-phosphate is about 0.1 μM to about 10 about 0.1 μM about 0.4 μM or about 0.2 μM to about 0.3 μM. In one embodiment, the concentration of vitamin A is about to about 10 about 0.3 to about 0.7 μM or about 0.4 μM to about 0.6 μM. In one embodiment, the concentration of vitamin E is about 0.1 μM to about 10 about 0.01 μM to about 0.04 μM or about 0.015 μM to about 0.03 μM. In one embodiment, the concentration of reduced glutathione about 0.1 μM to about 10 about 0.05 μM to about 0.4 μM or about 0.1 μM to about 0.3 μM. In one embodiment, the concentration of PQQ is about 0.1 μM to about 100 μM, e.g., about 1 μM to about 50 μM. In one embodiment, the concentration of NAC is about 0.1 mM to about 10 mM, e.g., about 0.1 mM to 50 mM. In one embodiment, the concentration of the complexes in the composition comprises about 0.1 μM to about 5 μM. In one embodiment, the concentration of the complexes in the composition comprises about 5 μM to about 50 μM. In one embodiment, the concentration of the complexes comprises about 50 μM to about 150 μM. Ratios of anti-oxidant to cyclodextrin may be 1:10, 1:2 or 1:5.

In one embodiment, the composition comprises one or more of ubiquinol, idebenone, MitoQ, vitamin E, vitamin C, ascorbate-2-phosphate, PQQ, NAC, reduced glutathione, or a C14-C18 saturated fatty acid, and optionally also a cyclodextrin, dextran, and amino acids, which composition is serum-free. In one embodiment, the ubiquinol in the composition is about 0.05 μM to about 100 μM, e.g., 0.05 μM to about 5 μM or about 7 μM to about 15 μM. In one embodiment, the concentration of vitamin C or ascorbate-2-phosphate is about 0.1 μM to about 10 about 0.1 μM about 0.4 μM or about 0.2 μM to about 0.3 μM. In one embodiment, the concentration of vitamin A is about 0.01 μM to about 10 about 0.3 μM to about 0.7 μM or about 0.4 μM to about 0.6 μM. In one embodiment, the concentration of vitamin E is about 0.1 μM to about 10 about 0.01 μM to about 0.04 μM or about 0.015 μM to about 0.03 μM. In one embodiment, the concentration of reduced glutathione about 0.1 μM to about 10 about 0.05 μM to about 0.4 μM or about 0.1 μM to about 0.3 μM. In one embodiment, the concentration of PQQ is about 0.1 μM to about 100 μM, e.g., about 1 μM to about 50 μM. In one embodiment, the concentration of NAC is about 0.1 mM to about 10 mM, e.g., about 0.1 mM to 50 mM. In one embodiment, the concentration of the complexes in the composition comprises about 0.1 μM to about 5 μM. In one embodiment, the concentration of the complexes in the composition comprises about 5 μM to about 50 μM. In one embodiment, the concentration of the complexes comprises about 50 μM to about 150 μM. In one embodiment, the composition further comprises a base medium and one or more of chondroitin sulfate, dextran, insulin, a buffer, non-essential amino acids, sodium bicarbonate. Ratios of anti-oxidant to cyclodextrin may be 1:10, 1:2 or 1:5.

In one embodiment, the composition comprises ubiquinol, idebenone, ubiquinol, MitoQ, vitamin E, vitamin C, ascorbate-2-phosphate, PQQ, NAC, palmitate, reduced glutathione, or a C14-C18 saturated fatty acid, and optionally a cyclodextrin, base medium, chondroitin sulfate, dextran, a buffer, non-essential amino acids, sodium pyruvate and sodium bicarbonate, which composition is serum-free. In one embodiment, the ubiquinol, idebenone or MitoQ in the composition is about 0.05 μM to about 5 μM or about 1 μM to about 15 μM. In one embodiment, the concentration of the complexes in the composition comprises about 0.1 μM to about 5 μM. In one embodiment, the concentration of the complexes in the composition comprises about 5 μM to about 50 μM. In one embodiment, the concentration of the complexes comprises about 50 μM to about 150 μM. Ratios of anti-oxidant to cyclodextrin may be 1:10, 1:2 or 1:5.

In one embodiment, the composition comprises ubiquinol, idebenone, ubiquinol, MitoQ, vitamin E, vitamin C, ascorbate-2-phosphate, PQQ, NAC, palmitate, reduced glutathione, or a C14-C18 saturated fatty acid, and optionally a cyclodextrin, dextran, and amino acids, which composition is serum-free. In one embodiment, the ubiquinol or MitoQ in the composition is about 0.05 μM to about 100 μM, e.g., 0.05 μM to about 5 μM or about 1 μM to about 15 μM. In one embodiment, the concentration of the complexes in the composition comprises about 0.1 μM to about 5 μM. In one embodiment, the concentration of the complexes in the composition comprises about 5 μM to about 50 μM. In one embodiment, the concentration of the complexes comprises about 50 μM to about 150 μM. In one embodiment, the composition further comprises a base medium. Ratios of anti-oxidant to cyclodextrin may be 1:10, 1:2 or 1:5.

In one embodiment, the composition comprises ubiquinol, idebenone or MitoQ, and optionally a cyclodextrin, base medium, chondroitin sulfate, dextran, a buffer, non-essential amino acids, sodium pyruvate and sodium bicarbonate, which composition is serum-free. In one embodiment, the ubiquinol or MitoQ in the composition is about 0.05 μM to about 100 μM, e.g., 0.05 μM to about 5 μM or about 1 μM to about 15 μM. In one embodiment, the concentration of the complexes in the composition comprises about 0.1 μM to about 5 μM. In one embodiment, the concentration of the complexes in the composition comprises about 5 μM to about 50 μM. In one embodiment, the concentration of the complexes comprises about 50 μM to about 150 μM. Ratios of anti-oxidant to cyclodextrin may be 1:10, 1:2 or 1:5.

In one embodiment, the composition comprises ubiquinol, idebenone or MitoQ, and optionally a cyclodextrin, dextran, and amino acids, which composition is serum-free. In one embodiment, the ubiquinol or MitoQ in the composition is about 0.05 μM to about 5 μM or about 1 μM to about 15 μM. In one embodiment, the concentration of the complexes in the composition comprises about 0.1 μM to about 5 μM. In one embodiment, the concentration of the complexes in the composition comprises about 5 μM to about 50 μM. In one embodiment, the concentration of the complexes comprises about 50 μM to about 150 μM. In one embodiment, the composition further comprises a base medium. Ratios of anti-oxidant to cyclodextrin may be 1:10, 1:2 or 1:5.

In one embodiment, the composition comprises highly water-dispersible submicron-supramolecular assemblies of an anti-oxidant, e.g., ubiquinol, prepared by mixing the anti-oxidant with a carrier, e.g., cyclodextrin (CD), for instance, γ-CD, at a molar ratio of, in one embodiment, 1:1 up to 1:20, for example, 1:5 to 1:10, which mixing is optionally under shearing force. Mixing may be aided by an aqueous-based solvent mixture. In one embodiment, the solution is formed of 1:10 to 10:1 absolute ethanol:water mixture, e.g., 1:2 to 2:1. In one embodiment, heat may be applied during the mixing process. In one embodiment, the heating temperature may be at 50° C. or above. In one embodiment, the mixing process employs a porcelain mortar and pestle. In one embodiment, the mixture is dried under vacuum in light-protected and moisture-protected conditions, to make white or off-white powder. In one embodiment, the powder is dispersed in deionized ultrapure water, wherein the particle size of the macromolecular assemblies is in the range of 50 to 900 micrometers, or the range of 100 to 500 micrometers. In one embodiment, the powder is added to media such as cell culture specific growth media, for example, corneal cell growth media and/or corneal storage media. In one embodiment, the final concentration of the anti-oxidant, e.g., ubiquinol, in the media is from about 10 to about 1000 micromolar, e.g., 50 to 250 micromolar. In one embodiment, the powder is added to cell culture media to reduce reactive oxygen species (ROS) generation, to increase oxygen consumption of cells, to prolong the storage time of stored corneal tissues, or any combination thereof. In one embodiment, the powder is added in the form of either a solid powder or a dispersion in sterile deionized and pyrogen-free water.

In one embodiment, the formulation is a topical eye drop to treat defects in the corneal epithelium or endothelium due to conditions such as Fuchs endothelial corneal dystrophy and diabetes mellitus prior to, during, or after ocular surgery. In one embodiment, the formulation is a tablet which can be added to a solution which in turn, can be employed to store corneas or portions thereof prior to transplant.

In one embodiment, the formulation is a topical eye drop for ophthalmic use in humans: to protect cellular health of the corneal endothelium, corneal epithelium, corneal nerves, and/or corneal stroma; to treat dysfunction or defects of the corneal endothelium, corneal epithelium, corneal nerves, and/or corneal stroma due to conditions such as diabetes and Fuchs endothelial cell dystrophy; in the preoperative, intraoperative, perioperative or postoperative settings for ocular surgeries such as cataract surgery, glaucoma surgery, or corneal surgery including transplantation; or any combination thereof. This formulation may be in the form of an ophthalmic solution or an ophthalmic suspension

In one embodiment, the formulation is an irrigating solution for ophthalmic use in humans to protect the corneal endothelium in the intraoperative setting for ocular surgeries such as cataract surgery, glaucoma surgery, intravitreal surgery, or corneal surgery including transplantation.

In one embodiment, the formulation is a tablet that can be added to a solution which, in turn, can be employed to store corneas or portions thereof prior to cornea transplant surgery.

The compositions described herein increase the short or intermediate term (corneal storage) and/or long term (e.g., post-transplant) health, function and/or viability of corneas, and corneal tissue including the corneal endothelium, corneal epithelium, corneal nerves, or corneal stroma. For example, the compositions described herein increase the health, function and/or viability of corneas, and corneal tissue including the corneal endothelium, corneal epithelium, and corneal stroma which are stored, after procuring and optionally culturing prior to transplant, particularly when stored for longer lengths of time, such as stored from 3 days, 5 day, 7 days, 10 day, 14 days, 21 days or more, relative to compositions that do not include the anti-oxidant and/or carriers described herein. Thus, the compositions may be employed for culturing, eye banking and the like.

### EXEMPLARY EMBODIMENTS

In one embodiment, a corneal preservation composition comprising an amount of about 0.05 μM to about 15 μM ubiquinol, idebenone or MitoQ, about 0.1 μM to about 10 μM vitamin C, 0.05 μM to about 10 μM vitamin A or vitamin E, about 0.1 μM to about 10 μM ascorbate-2-phosphate, about 0.1 to about 100 μM pyrroloquinoline quinone (PQQ), about 0.1 mM to about 10 mM N-Acetyl-L-cysteine (NAC), 0.1 μM to about 750 μM palmitate, or 0.1 to about 10 μM reduced glutathione, is provided. In one embodiment, the composition comprises ubiquinol. In one embodiment, the composition comprises an amount of chondroitin sulfate or one or more omega 3 fatty acids, e.g., docosahexaenoic acid (DHA), eicosapentaenoic acid (EPA) and/or alpha-linolenic acid. In one embodiment, the composition comprises one or more carriers, e.g., cyclodextrin, polyethylene glycol (PEG), PEG dodecyl ether (Brij L4®), PEG hexadecyl ether (Brij 58®), lipid-based solubilizers like Labrafil® and Labrafac®, pluronics, e.g. Pluronic F68 (Poloxamer 188), polysorbate 80 and 20 or lipid nanoparticles, which form complexes with one of more of the other components, e.g., ubiquinol, idebenone, PQQ or NAC. In one embodiment, the composition is formulated for drops or injection. In one embodiment, the composition comprises is a tablet or a lyophilized powder. In one embodiment, the composition comprises a full thickness cornea. In one embodiment, the composition comprises a partial thickness cornea. In one embodiment, the composition comprises corneal endothelium. In one embodiment, the amount is effective to decrease corneal endothelial cell death, decrease apoptosis or decrease necrosis, or any combination thereof. In one embodiment, the complexes are about 200 to about 400 nm, about 100 to about 300 nm, about 300 to about 500 nm in diameter, or up to about 1000 nm in diameter. In one embodiment, the composition comprises about 7 μM to about 15 μM ubiquinol. In one embodiment, the composition comprises a base medium and one or more of chondroitin sulfate, dextran, insulin, a buffer such as HEPES buffer, non-essential amino acids, or sodium bicarbonate.

Further provided is a method of making complexes of one or more anti-oxidants comprising ubiquinol, idebenone, NAC, PQQ, vitamin A, vitamin C, ascorbate-2-phosphate, reduced glutathione, vitamin E, or a C14-C18 saturated fatty acid, and a carrier, comprising: combining an amount of the one or more anti-oxidants and an amount of a carrier under low light and low oxygen conditions so as to form complexes of about 100 to about 500 nm in diameter. In one embodiment, the molar ratio of the anti-oxidant to the carrier is from x:y, where x and y are independently any integer between 1 and 1000, e.g., 1:1 to 1:1000, 2:1 to 1:10 or 3:1 to 1:20. In one embodiment, the molar ratio of the anti-oxidant to the carrier is 2:1 to 1:20. In one embodiment, the molar ratio of anti-oxidant to cyclodextrin is about 1:15, 1:10, 1:5, or 1:20.

Also provided is a method of preserving a cornea, corneal tissue or corneal endothelium, or other tissue of a mammal, comprising: providing a cornea, corneal tissue or corneal endothelium, or other tissue of a mammal; and combining the cornea, corneal tissue or corneal endothelium or other tissue and the composition disclosed herein. In one embodiment, the tissue is stored for up to 21 days at 2-40, e.g., 2-8, ° C. prior to transplant. In one embodiment, the tissue is stored for up to 14 days at 2-40, e.g., 2-8, ° C. prior to transplant. In one embodiment, the tissue comprises corneal endothelium, corneal epithelium, corneal keratocytes, corneal stroma, corneal nerves, conjunctival epithelium, conjunctival stroma, Tenon's capsule, trabecular meshwork, corneoscleral angle, lens epithelium, or lens.

In addition, a method of treating corneal endothelium, corneal epithelium, corneal keratocytes, corneal stroma, corneal nerves, conjunctival epithelium, conjunctival stroma, Tenon's capsule, trabecular meshwork, corneoscleral angle, lens epithelium, or lens tissue in a mammal is provided. The method includes administering to a mammal in need thereof an effective amount of the composition. In one embodiment, the mammal is a human. In one embodiment, the mammal is a diabetic. In one embodiment, the mammal has an ocular disease. In one embodiment, the human is a candidate for ocular surgery. In one embodiment, the surgery is cataract surgery, keratoplasty, removal of corneal tissue or lesions, ocular surface surgery including but not limited to pterygium surgery and lesion biopsies, vitreoretinal surgery, or glaucoma surgery. In one embodiment, the human has had ocular surgery. In one embodiment, the composition is administered during ocular surgery. In one embodiment, the mammal has Fuchs endothelial corneal dystrophy.

The invention will be further described by the following non-limiting examples.

### Example 1

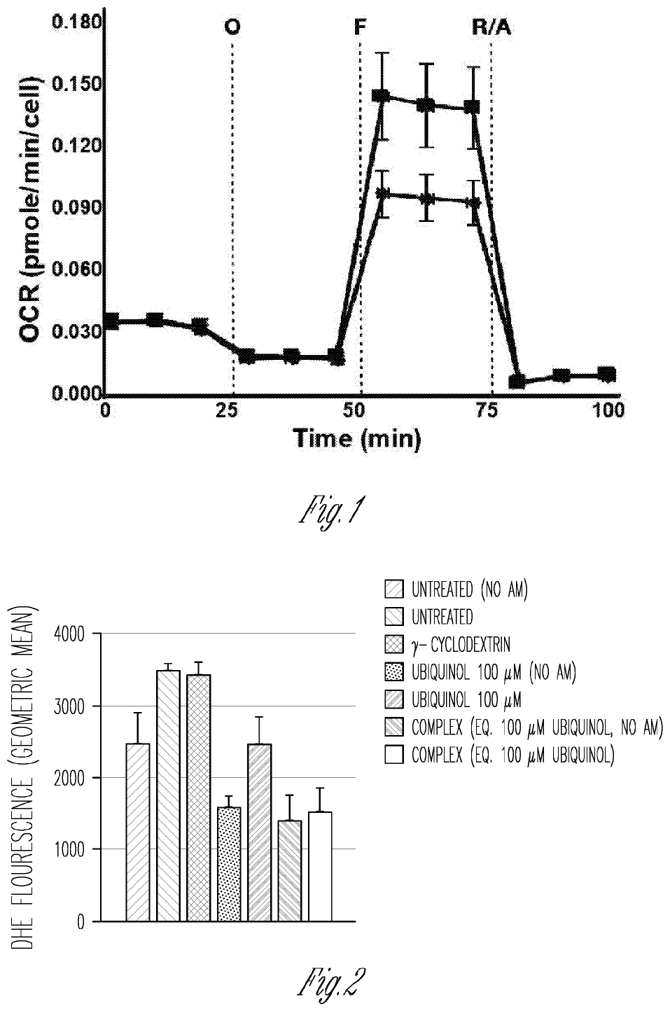

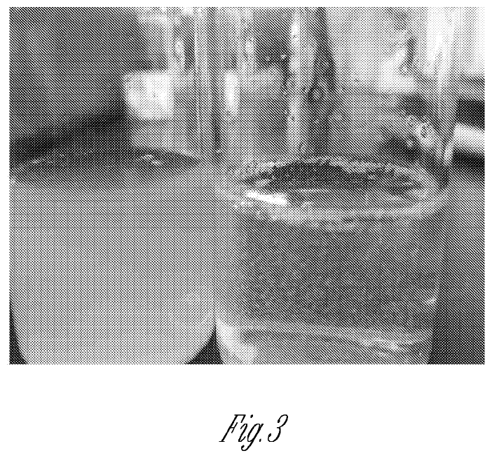

When ubiquinol is supplemented to corneal storage medium, it was found that mitochondrial respiration was significantly increased, compared to cells incubated with non-supplemented storage medium. Specifically, in corneal endothelial cells supplemented with ubiquinol, proton leak was increased by 34% (p=0.046), maximum respiration was increased by 97% (p=0.003), and spare respiratory capacity was increased by 133% (p<0.001). CEC cell death was decreased in storage due to ubiquinol supplementation. FIG. 1 shows increased oxygen consumption rate (OCR) of healthy corneal cells supplemented with ubiquinol, indicating higher spare respiratory capacity, compared to non-supplemented cells.

### Example 2

Fifty mg of ubiquinol were mixed by geometric mixing with 750 mg of γ-CD (molar ratio 1:10), then levigated using a mortar and a pestle with slow addition of water: ethanol mixture (1:1). The total volume of the ethanolic mixture is not more than 5 mL. The whole levigation/trituration may be for about 1 h in the darkness and takes place under the fume hood to minimize oxygen exposure. Water that is used is flushed with nitrogen to minimize dissolved oxygen. Trituration/levigation continues until the composition is almost dried. It is then thoroughly dried under vacuum and light protection. This composition is then added to Optisol GS and other cell culture media like Dulbecco's Modified Eagle medium (DMEM) at a concentration equivalent to 100 μM ubiquinol.

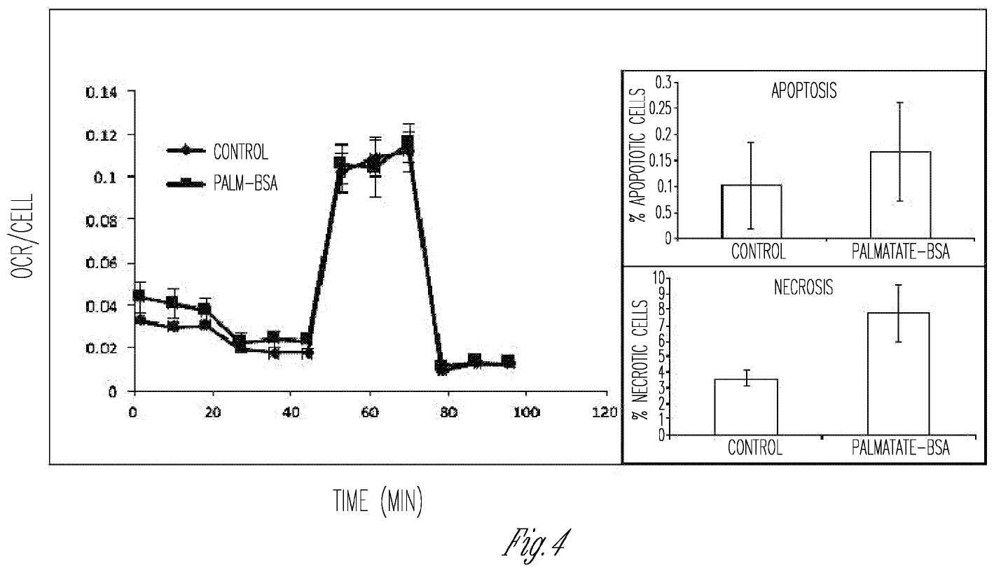

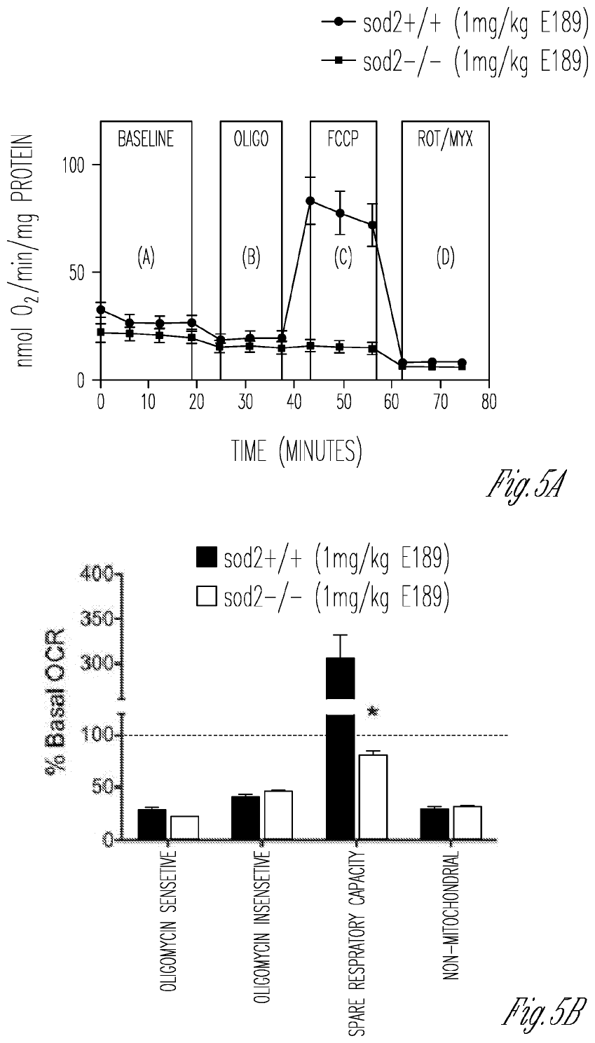

Unexpectedly, as described herein, it found that compositions comprising ubiquinol, kneaded with γ-CD, with or without heat, under light- and oxygen-protected conditions, could completely abolish the ROS generation induced by antimycin-A (AM) in human endothelial lung cancer cells (A549), while free ubiquinol was unable to inhibit ROS generated by the same concentration of AM (FIG. 2). Thus, compositions having certain amounts of an anti-oxidant and a carrier may increase mitochondrial respiration and ECD of human donor corneal endothelial cells and/or primary corneal endothelial cells, and are hence expected to markedly decrease cell loss during corneal tissue preparation prior to corneal graft procedure like DSAEK. In addition, these compositions showed high dispersibility in water, compared to free ubiquinol (FIG. 3), and appear to form submicron assemblies in the range of 200-600 nm, as shown by dynamic light scattering.

### Example 3

To determine if adding the anti-oxidant Coenzyme Q10 (CoQ10 or ubiquinol) to donor cornea storage media enhances the metabolic function of corneal endothelial cells (CECs) and/or decreases overall cell death in storage, the same quantities of ubiquinol and γ-CD are mixed together as mentioned above. The mixture is heated to 50° C. during mixing. The vacuum dried mixture is then added in the same concentration as example 1 to cell culture media in order in order to effectively inhibit the ROS levels in these cells.

### Methods

Human corneal tissue pairs were obtained by Iowa Lions Eye Bank (ILEB) from nondiabetic donors 60-75 years old and stored in Optisol GS (Bausch+Lomb, Irvine, Calif.) at 4° C. following procurement in accordance with Eye Bank Association of America and ILEB policies and procedures. For 5 days prior to testing, but within 9 days of procurement, one stored tissue from a corneal pair was treated with 10 μg/mL CoQ10, while the mate tissue was treated with diluent only as a control. Descemet membrane and endothelial cell punches were collected and mounted onto the bottom of a Seahorse assay plate (cells facing upward). Mitochondrial respiration was assayed by measuring oxygen consumption using the Seahorse XFe24 Extracellular Flux Analyzer (Agilent Technologies, Santa Clara, Calif.) over 120 minutes (9-minute intervals). Punches were labeled with a nuclear counterstain (DAPI) and remaining tissues were mounted onto slides and labeled with 488A-Annexin V, Ethidium Homodimer III, and Hoechst 33342 (Apoptotic, Necrotic, and Healthy Cells Quantification Kit; Biotium, Fremont, Calif.) to assay cell health. Nuclei were counted in each punch to normalize respirometry data. Immunohistochemistry densitometry was measured using ImageJ software.

### Results

In total, 14 paired corneas were tested. Three different aspects of mitochondrial respiration were affected by CoQ10 treatment: proton leak was increased 34% (p=0.046), maximal respiration was increased 97% (p=0.003), and spare respiratory capacity was increased 133% (p<0.001). Corneal endothelial cell necrosis was not changed, however, apoptosis was reduced 29% in treated cells (p=0.09).

### Conclusions

In this series, Coenzyme Q10 increased corneal endothelial cell mitochondrial respiration and prevented cells from dying in storage. Findings indicate that Optisol GS supplemented with CoQ10 may reduce presurgical cell death and functional decline related to tissue storage. Further studies determine the dosing strategy during storage as well as the cytoprotective effects on cell density after endothelial keratoplasty.

### Example 4

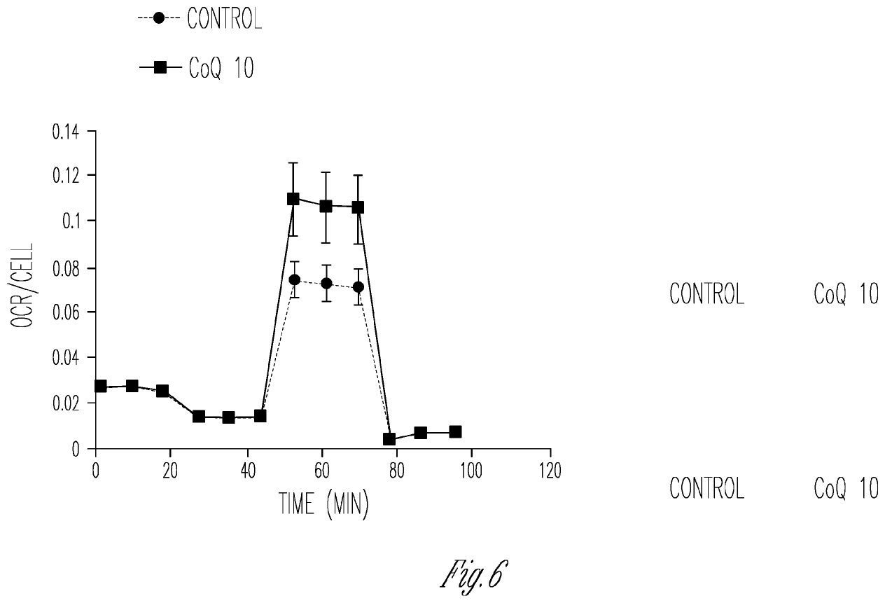

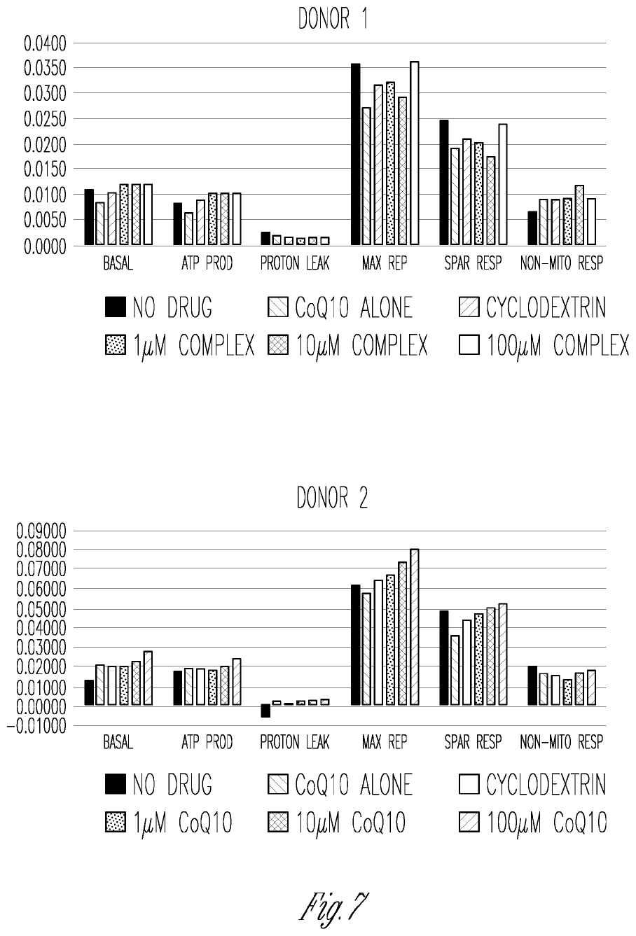

The effects of adding palmitate-BSA were determined. For 10 paired corneas, one was treated with palmitate-BSA and one was treated with BSA only, for 5 days. The only significant respiration change found was in proton leak, with treated cells having a 45% higher proton leak than untreated control cells (p-value=0.031). Looking at the overall levels of apoptosis and necrosis however, it was found that the treated cells also had a 116% higher amount of necrosis (p-value=0.0353) and 67% higher amount of apoptosis (p-value=0.0286). This indicates that increases in proton leak due to palmitate-BSA exposure is actually toxic to the cells. See FIG. 4.

### Example 5

It was determined if adding a mitochondria performance enhancing supplement anti-oxidant (coenzyme Q10, including its active form ubiquinol) to cornea storage medium enhances the metabolic function of the corneal endothelial cells and decreases their amount of cell death in storage. Increasing the metabolic function of cells in storage and mitigating cell death as well would boost cell health, making the tissue better equipped to handle the stress of both storage and transplant. The result would then be better performing tissue post-transplant, with an overall reduction in graft failure. Also, we aimed to specifically study the effects on mitochondrial ROS and depolarization by soluble coenzyme Q10 (cyclodextrin-coQ10) using human corneal endothelial cell cultures. Cytoprotection against ROS and cellular stress as well as clinically through ophthalmic preparations (topical drops, injections) in patients would protect cells against stressors ranging from disease to prior intraocular surgeries. The goal is to reduce corneal edema and decompensation that result from endothelial dysfunction. Both of these strategies would result in endothelial cells resistant to ROS mediated damage with an overall reduction in the need for transplant surgery and better performing transplanted corneal endothelial cells with an overall improvement in graft survival.

Sod2 null mice demonstrate impaired mitochondrial function as a result of mitochondrial oxidative stress. FIG. 23 shows the OCR results from the Sod2 null mice. As shown, the spare capacity is reduced when mitochondrial ROS mitigatory enzyme Sod2 is absent. This is the exact function that is bolstered by co-Q10 in cornea studies (FIG. 20).

### Methods

Human corneas were obtained by Iowa Lions Eye Bank (ILEB) from nondiabetic donors 60-75 years old and stored in Optisol GS (Bausch+Lomb, Irvine, Calif.) at 4° C. following procurement in accordance with Eye Bank Association of America and ILEB policies and procedures. Endothelial cells were isolated and cultured in Seahorse XFe96 well plates until they reached confluency. Purity of cell cultures were confirmed with anti-zonula occludens 1 (ZO-1) labeling of cellular tight junctions. Once confluent, cells were treated with different concentrations of cyclodextrin-coenzyme Q10 complex in culture (1 μM, 10 μM, or 100 μM) uncomplexed coenzyme Q10 (100 μM), cyclodextrin alone, or diluent control. Mitochondrial respiration was assayed by measuring oxygen consumption using the Seahorse XFe96 Extracellular Flux Analyzer (Agilent Technologies, Santa Clara, Calif.) over 120 minutes (9-minute intervals). Wells were labeled with a nuclear counterstain (DAPI) and nuclei were counted in each well to normalize respirometry data.

Immortalized human corneal endothelial cells were grown in 96 well plates until reaching confluency and then treated with 1 μM or 100 cyclodextrin-coenzyme Q10 complex, or diluent alone for a control. Cells were incubated for 48 hours and then assayed for mitochondrial ROS quantification using a fluorescent plate reader kit (ab219943; Abcam, Cambridge, Mass.).

### Results

Using cells cultured from donor corneas (primary cultures), it was found that coQ10 is not soluble and in fact reduces mitochondrial spare respiratory capacity. It was also found that cyclodextrin alone also reduces the mitochondrial spare respiratory capacity, as well as other measures of mitochondrial function. Three concentrations of cyclodextrin-coQ10 complexes were tested on immortalized human corneal endothelial cells. Although the results were variable, the results trend to show that high concentrations of the complex 100 μM rescues the reduction of mitochondrial function caused by cyclodextrin alone, however, not always does the effect surpass that of the control (untreated) cells.

In addition, immortalized human corneal endothelial cells with cyclodextrin-coQ10 were tested or ROS and mitochondrial depolarization using plate reader quantification assays. Two concentrations of the complex were studied, 1 μM and 100 μM. The lower concentration of complex significantly reduced levels of ROS (P<0.001), while the higher concentration did not (P=0.37). See FIG. 24.

### Conclusions

Complexed coQ10 with cyclodextrin may be employed for different applications including transplant tissue storage medium supplementation, ophthalmic topical drops, ophthalmic injections, etc.

Coenzyme Q10 is not only a safe addition to cornea storage medium, but it enhances the function of the corneal endothelial cell mitochondria and decreases their overall death. Coenzyme Q10 is a supplement that may enhance transplant tissue and reduce graft failure overall in the future. Also, soluble coQ10 developed for clinical use for ROS affected conditions (diabetes, prior surgeries) in the form of topical drops and injections may reduce the need for transplants in general. Both applications will bolster corneal endothelial cell health by reducing susceptibility to ROS mediated dysfunction, altogether preventing cell loss, vision loss from corneal edema and improving transplant survival.

### Example 6

### Results

7 different components of mitochondrial related respiratory events were examined: basal respiration, ATP production, proton leak, maximal respiration, spare respiratory capacity, non-mitochondrial respiration, and coupling efficiency. The output of these assays were measured as oxygen consumption rate, normalized to cell densities of the corneal endothelial tissues separately. An assay to assess overall levels of apoptosis and necrosis, separately, relative to live cells.

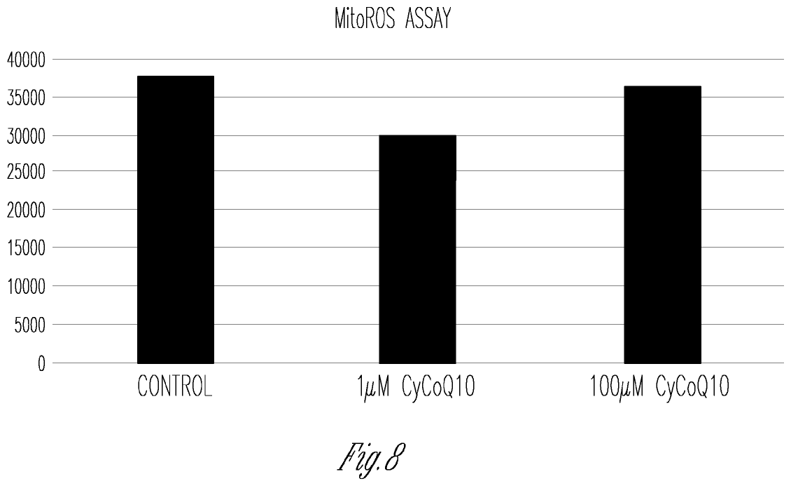

The effects of adding 10 μM coenzyme Q10 to the storage medium were examined. 13 paired corneas were tested, half incubated with co-Q10 and the mates treated with diluent only as a control, for 5 days. Three different aspects of mitochondrial respiration were affected by treatment: proton leak was increased 34% with co-Q10 treatment (p-value=0.0458), maximal respiration was increased 97% with treatment (p-value=0.003), and spare respiratory capacity was increased 133% (p-value<0.001). Overall corneal endothelial cell necrosis did not change (p-value=0.85) and apoptosis was 29% lower in treated cells (p-value=0.09). This indicates that coenzyme Q10 boost the mitochondrial function of the endothelial cells, but it also prevented the cells from dying in storage (FIG. 6).

Next, the effects of lower doses (0.5, 1, 5, and 7.5 μM) on tissues in storage were examined and it was found that 1 μM protected not only against apoptosis, but also against necrosis. This concentration did not show a bolstering of mitochondrial spare respiratory capacity function as seen with the 10 μM dose however. It did show the largest increase in non-mitochondrial respiration. Also, there was no increase in proton leak at 1 μM. This is a positive finding since proton leak may be an indicator of early depolarization. At this point, it appears that coQ10 supplementation may provide two protective effects, at different concentrations. At the lower concentration, it appears to protect overall cell health, decreasing both apoptosis and necrosis, but not alter mitochondrial respiration. At the higher dose, apoptosis is reduced and mitochondrial spare capacity is bolstered. However, also at the higher dose, proton leak increases.

### Cell Culture Assays

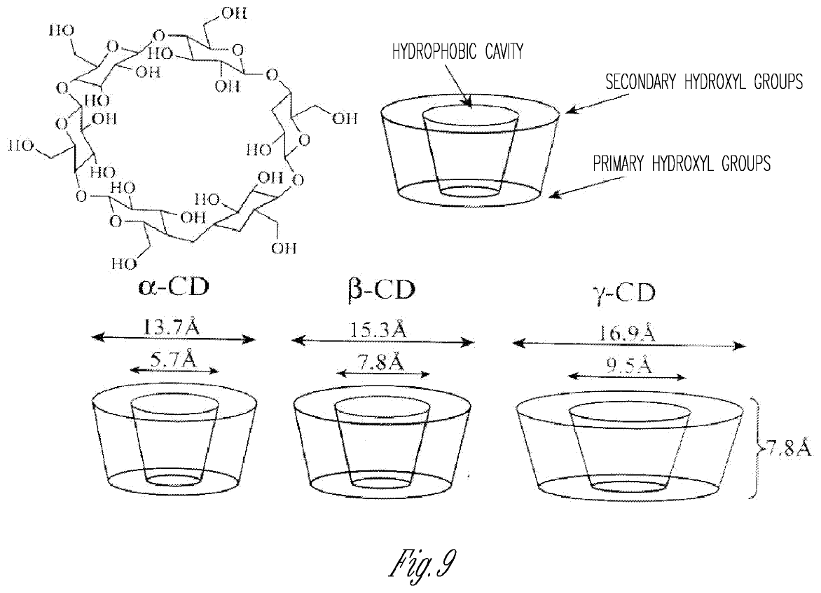

Using cells cultured from donor corneas (primary cultures), it was found that co-Q10 is not soluble and in fact reduces mitochondrial spare respiratory capacity. It was also found that cyclodextrin alone also reduces the mitochondrial spare respiratory capacity, as well as other measures of mitochondrial function. Three concentrations of cyclodextrin-coQ10 complexes were tested on immortalized human corneal endothelial cells. Although the results were variable, the results trend to show that high concentrations of the complex 100 μM rescues the reduction of mitochondrial function caused by cyclodextrin alone, however, not always does the effect surpass that of the control (untreated) cells. See FIG. 7, showing examples of primary culture Seahorse metric results.

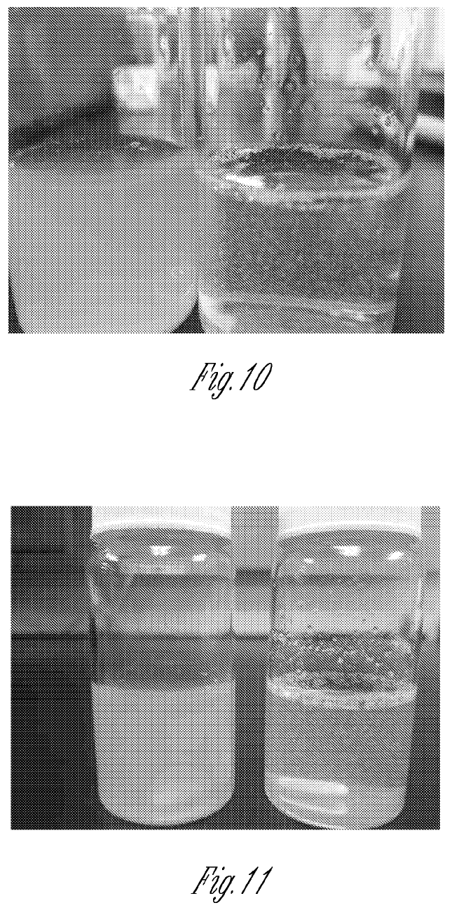

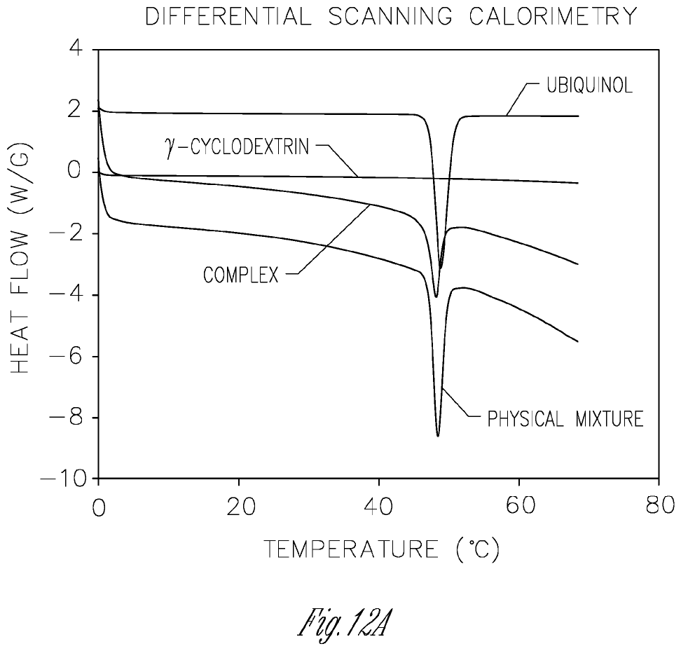

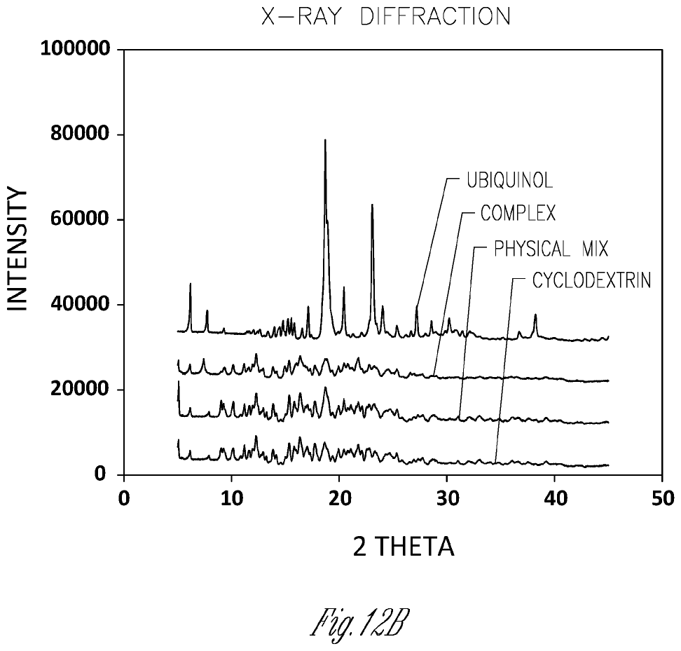

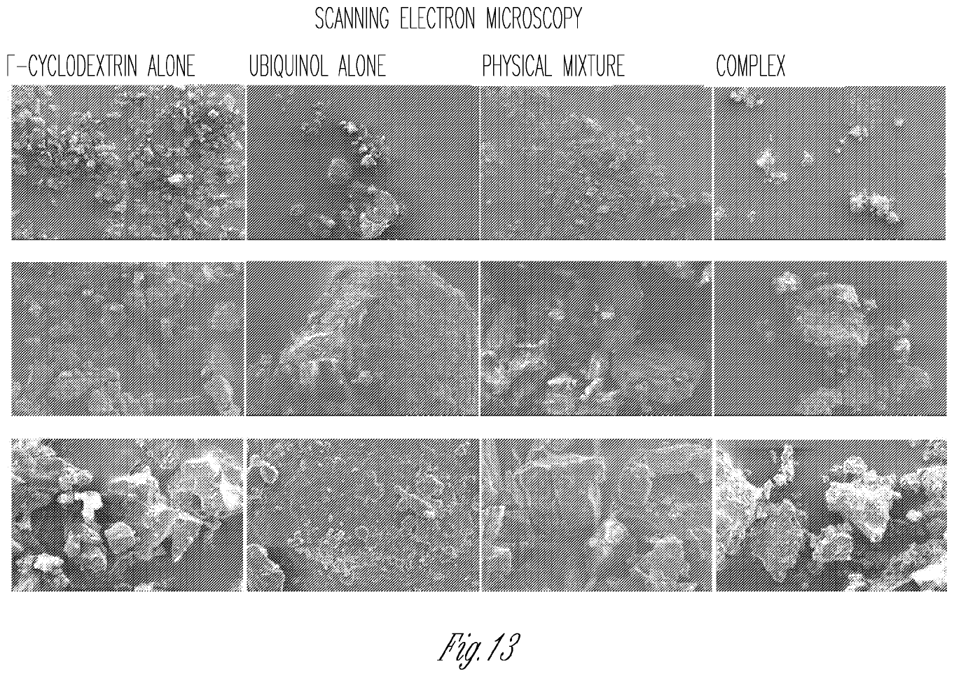

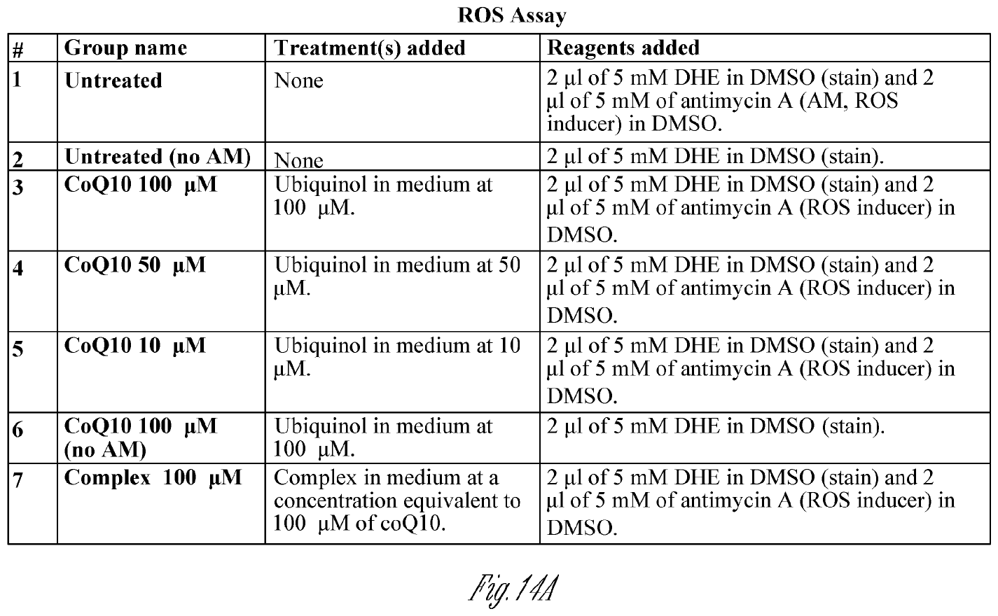

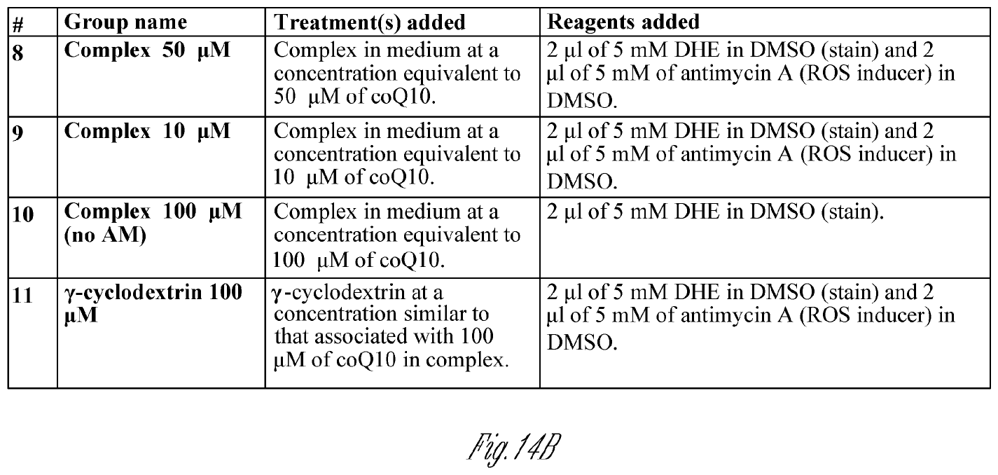

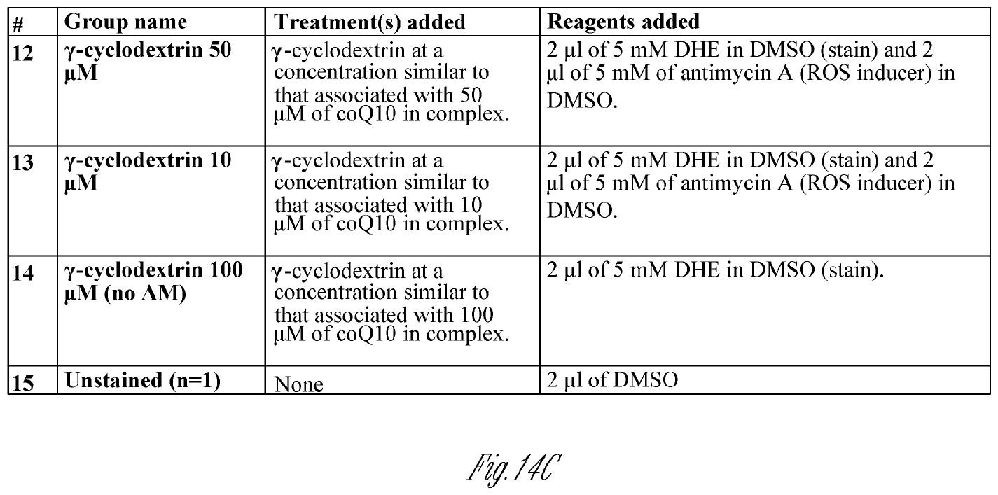

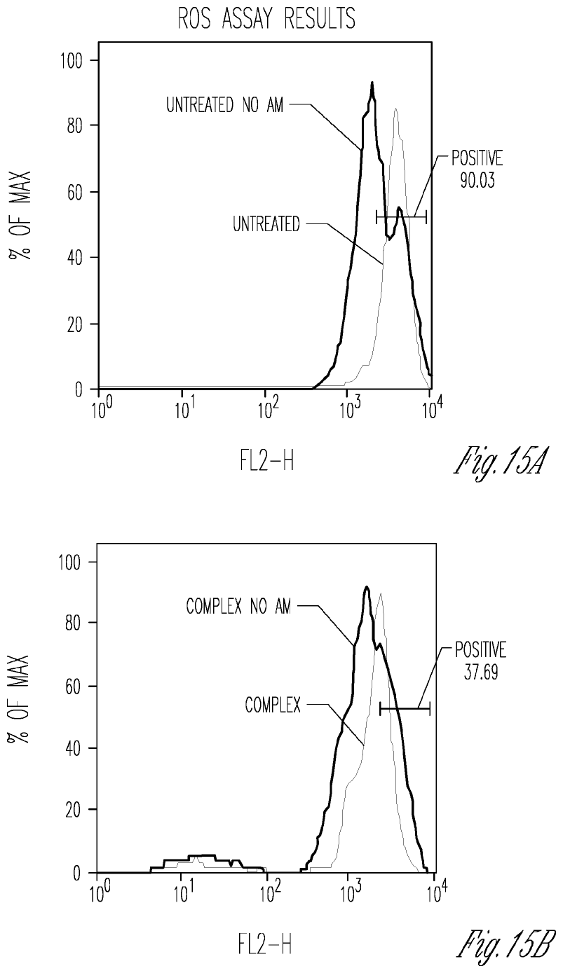

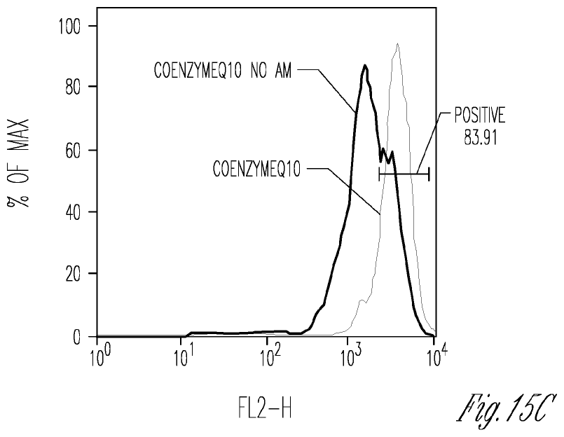

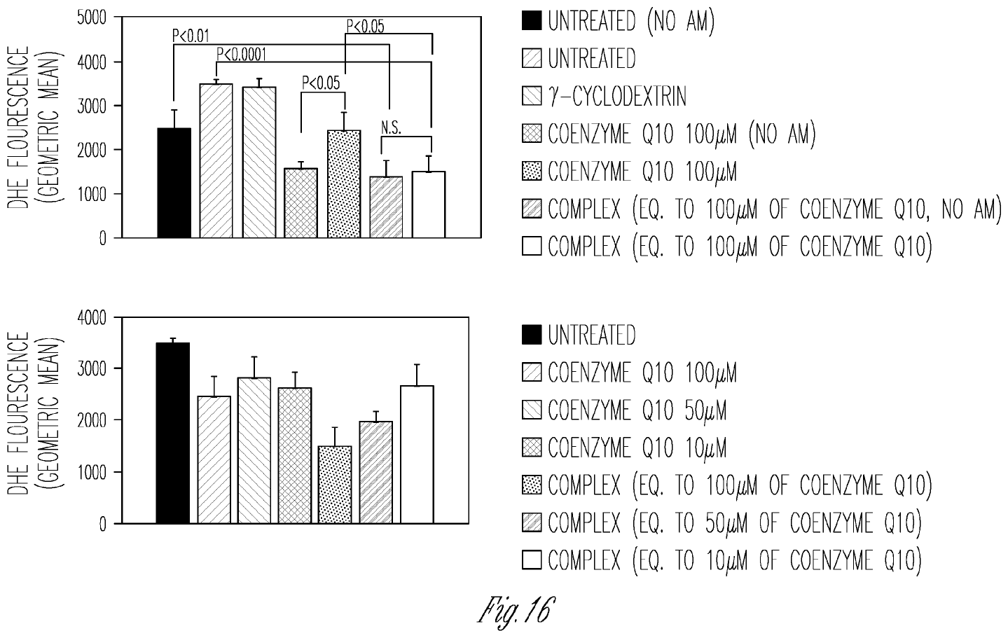

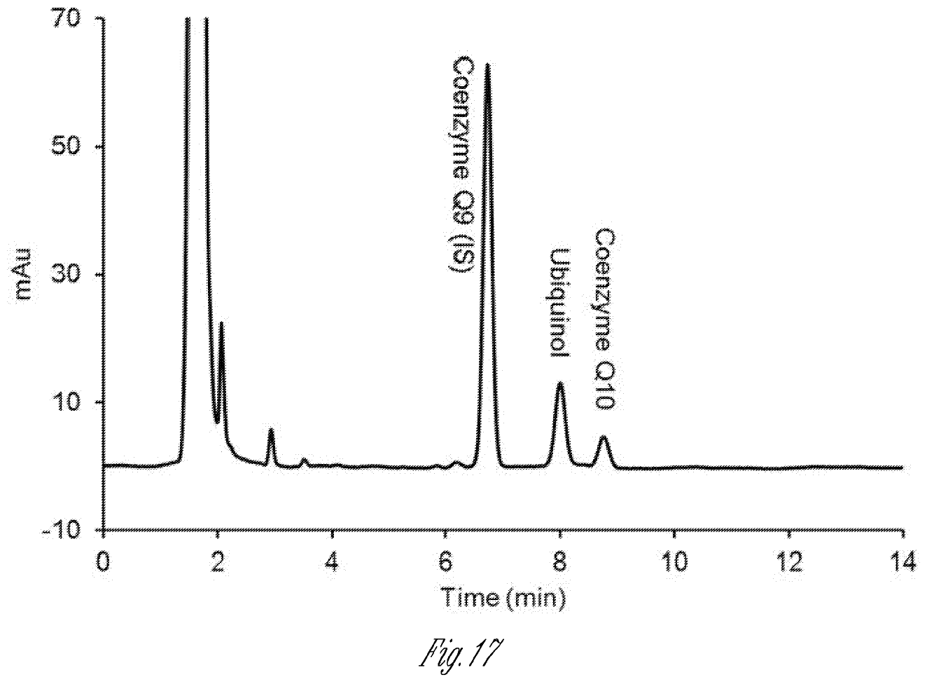

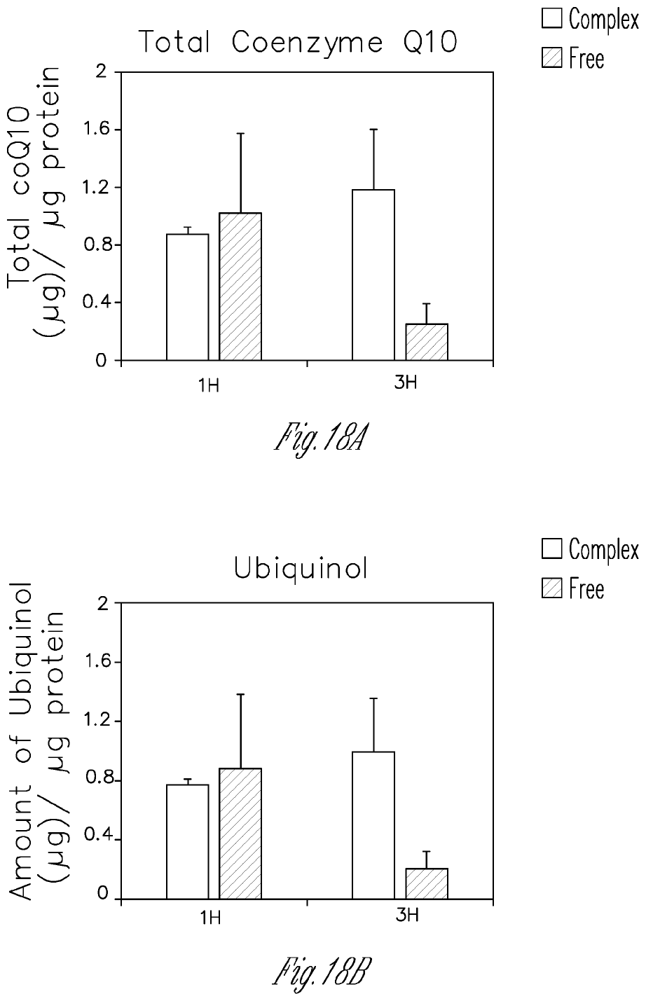

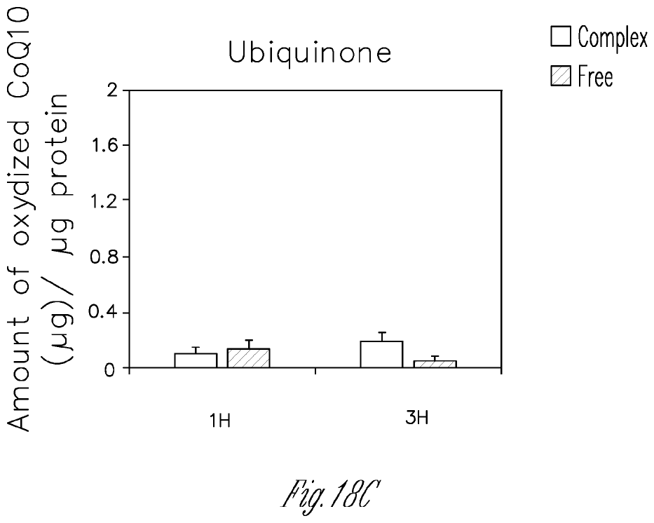

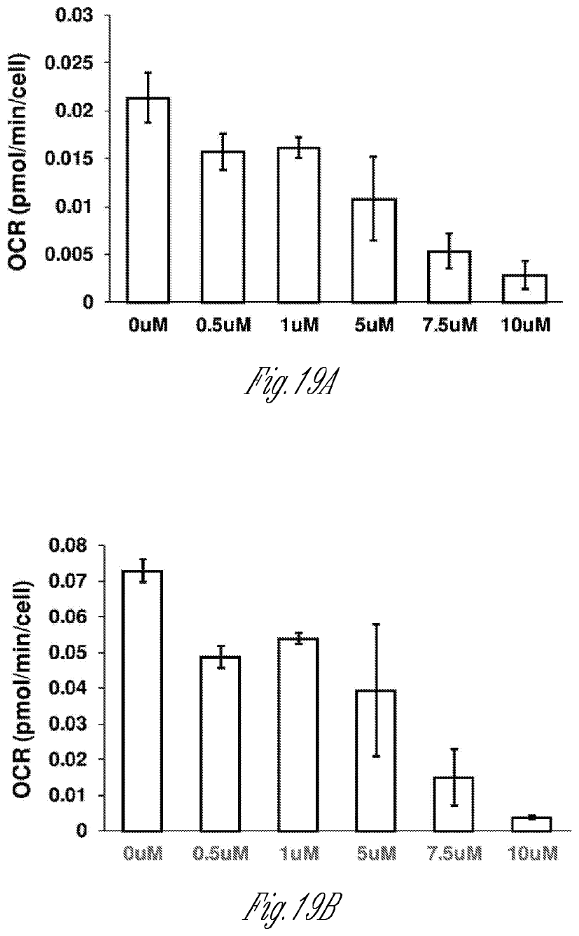

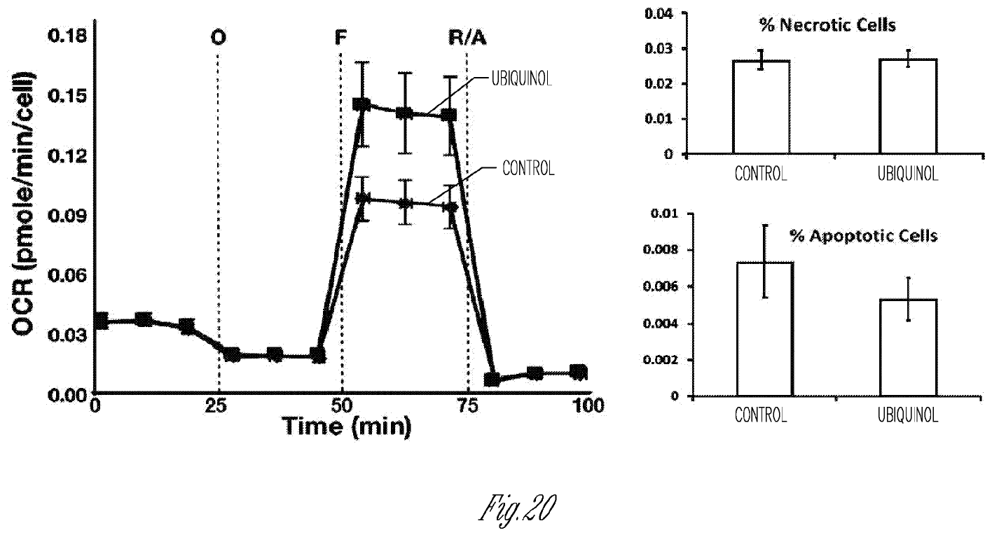

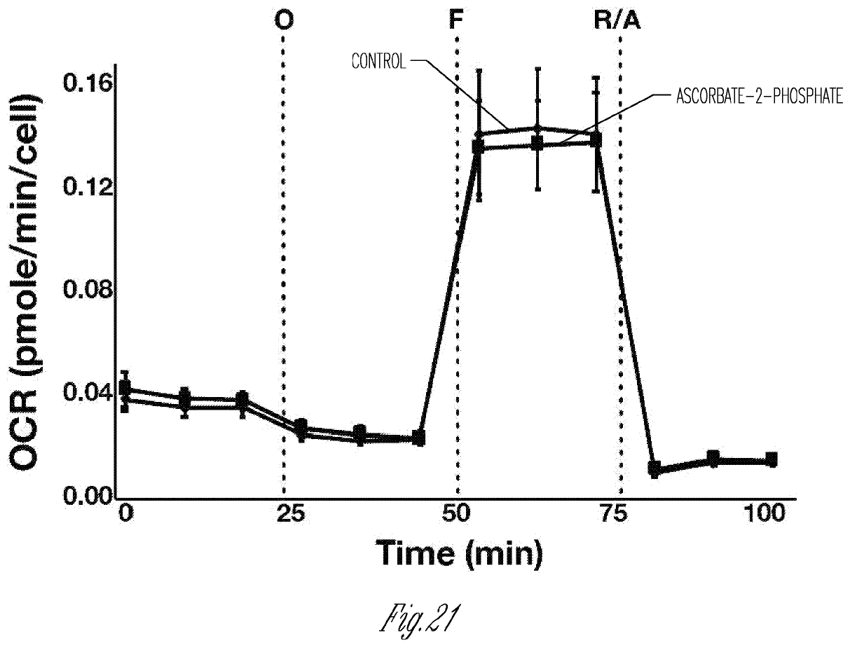

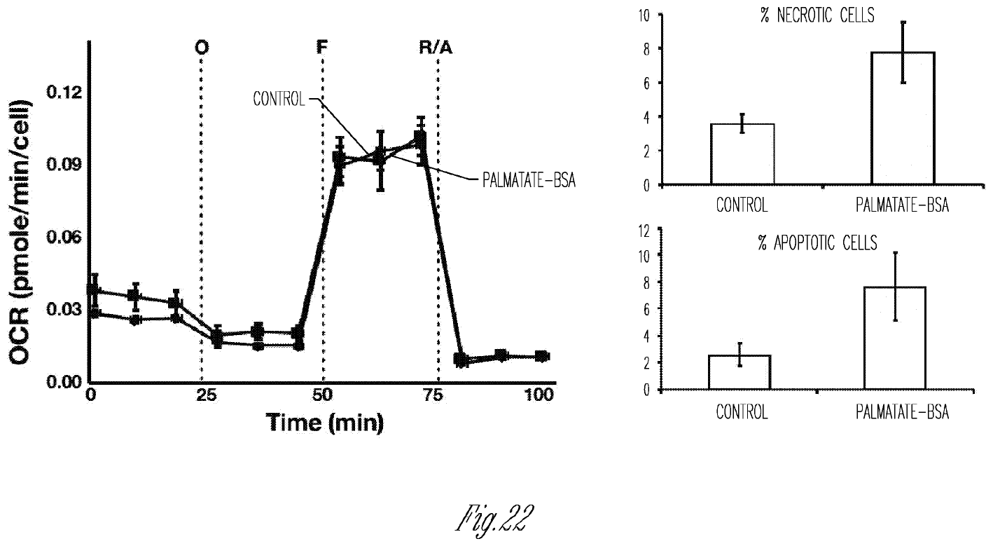

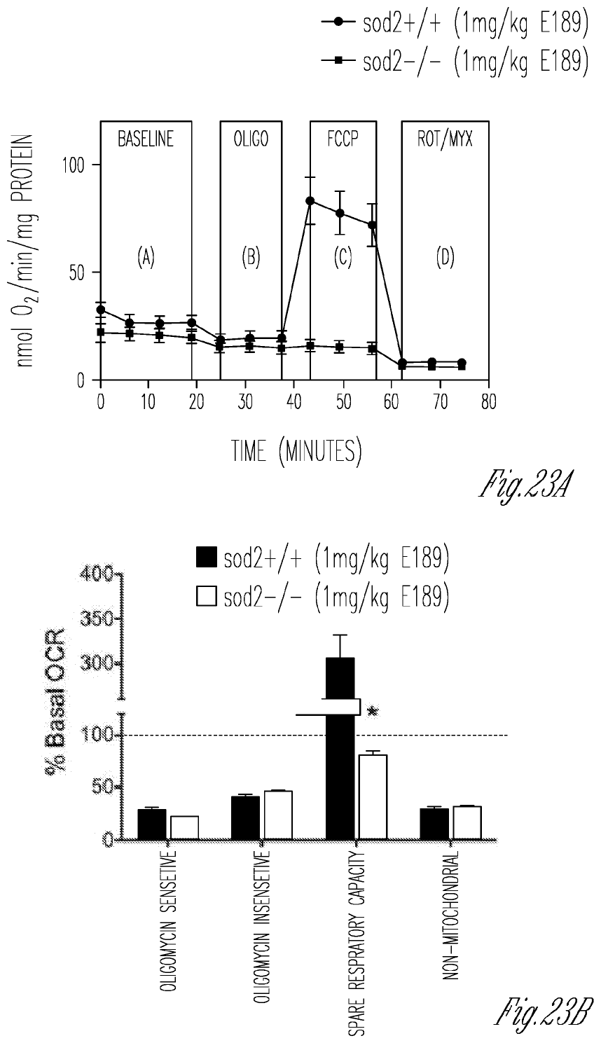

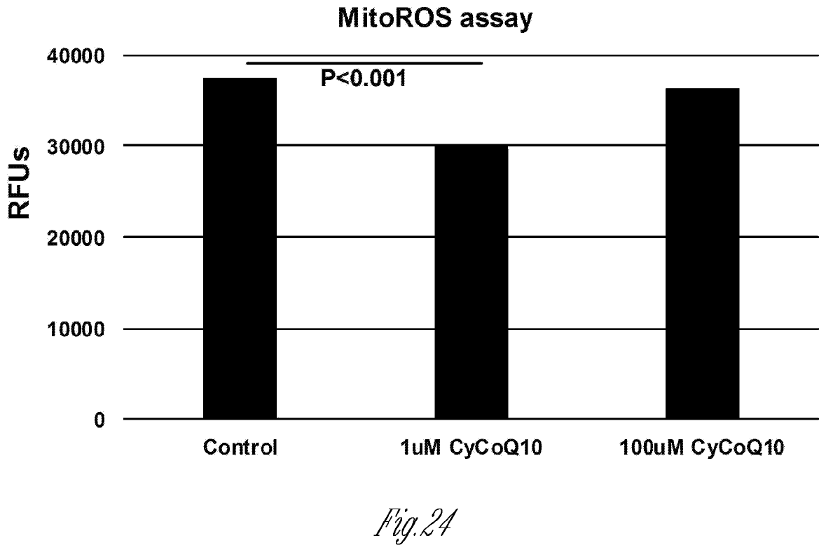

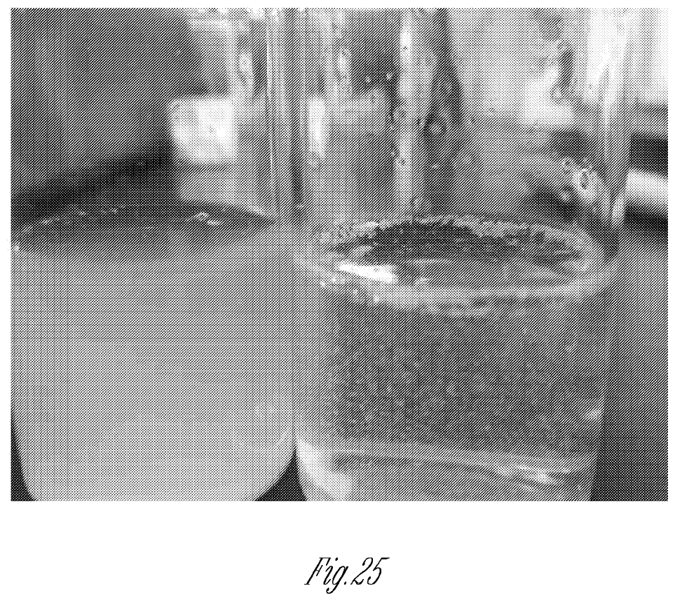

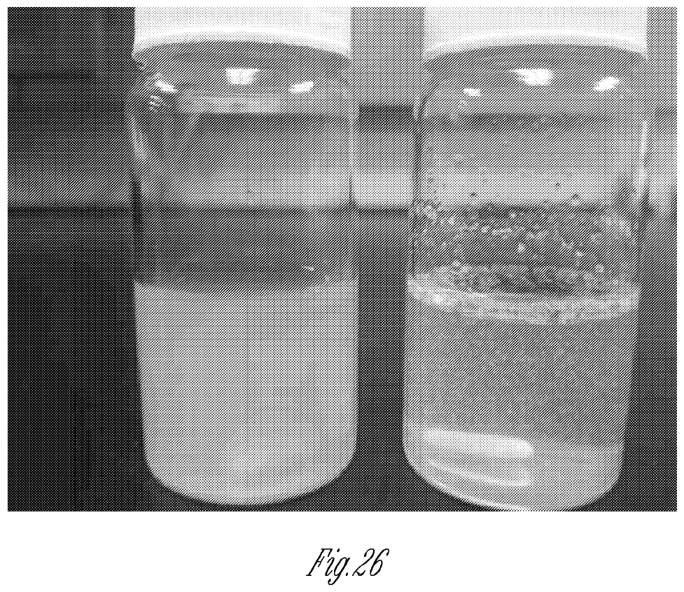

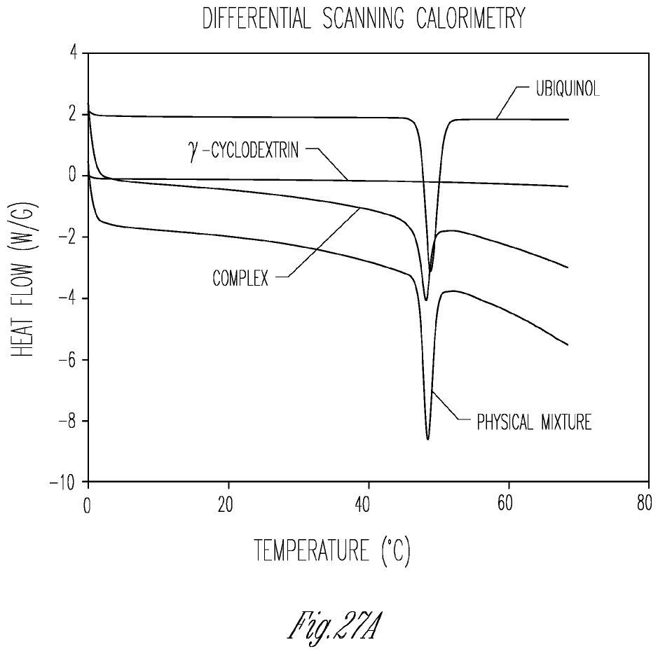

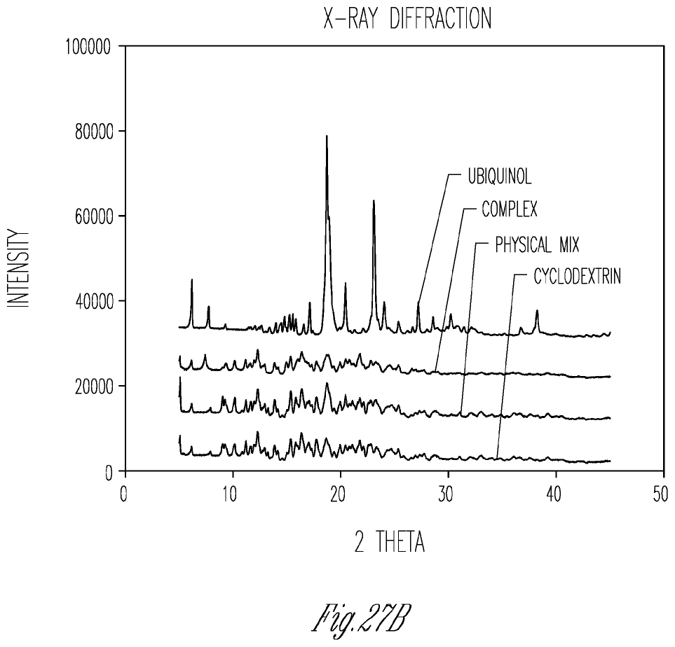

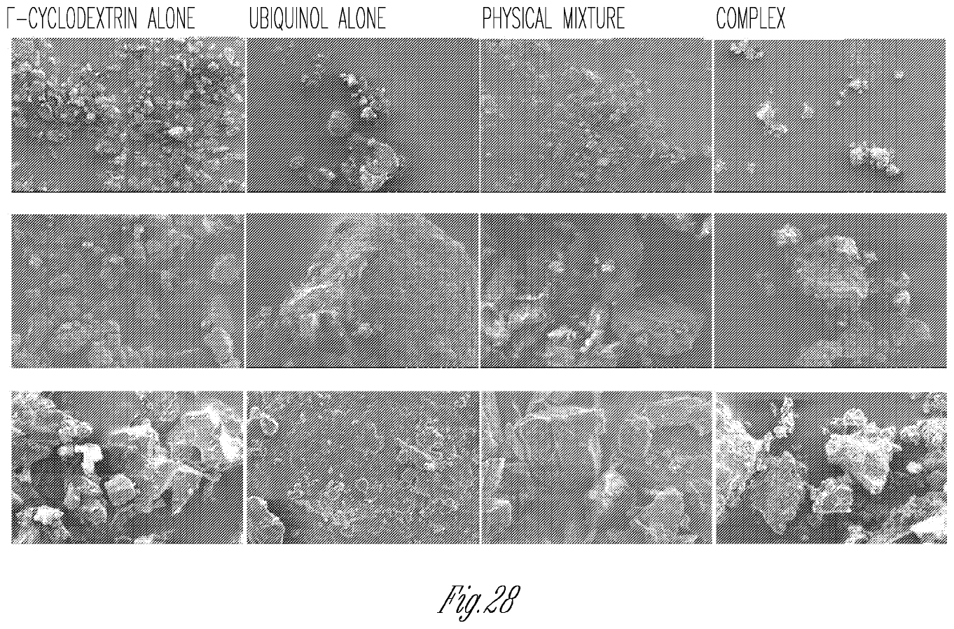

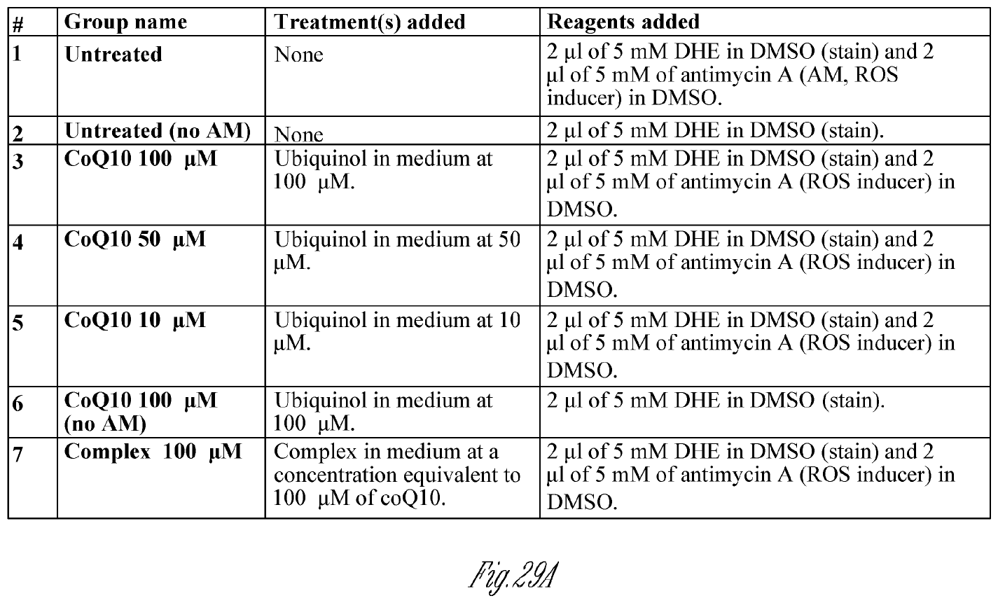

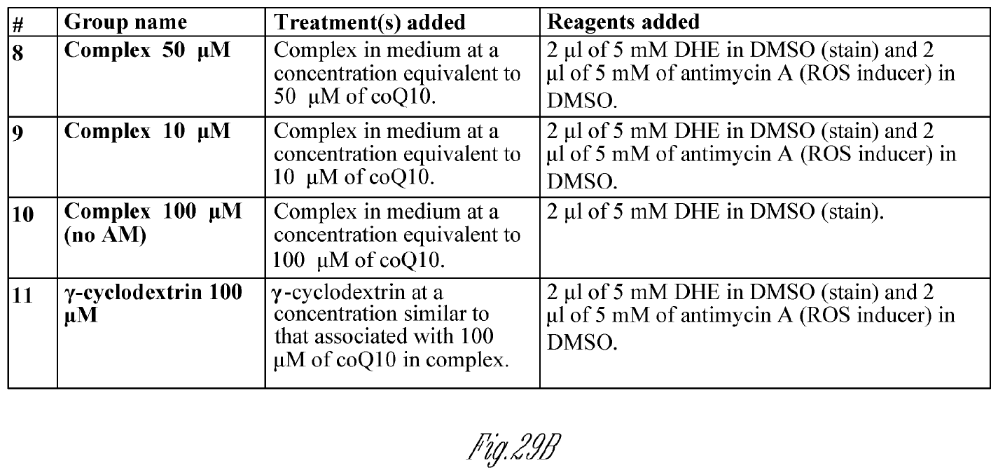

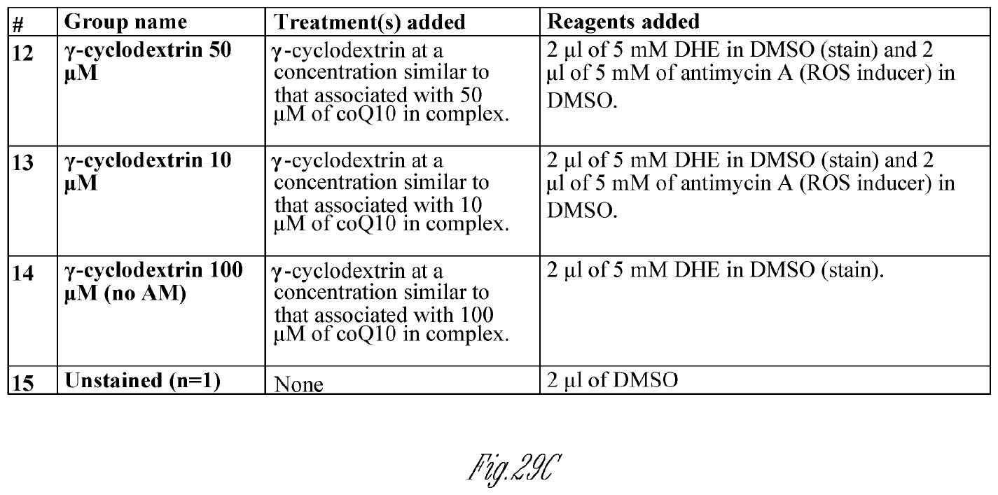

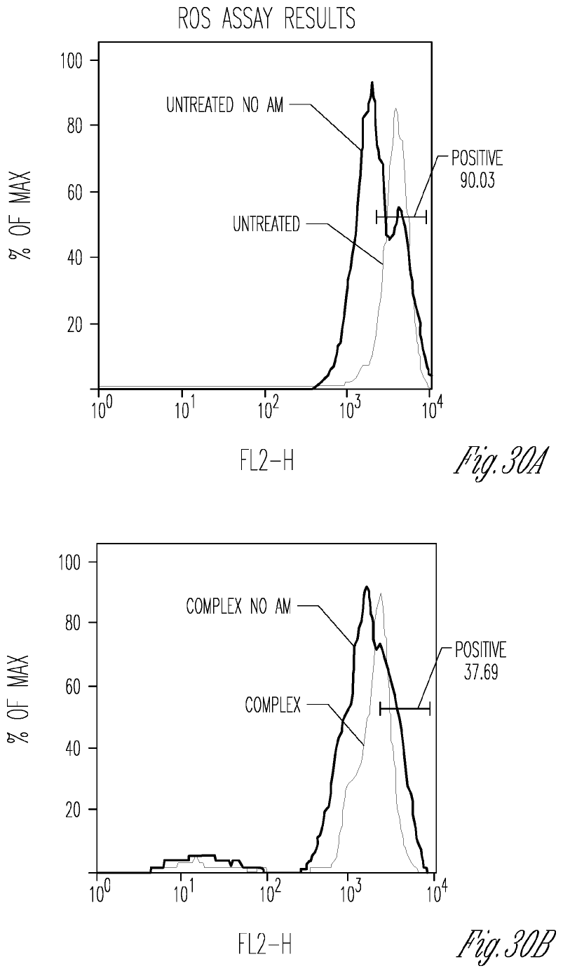

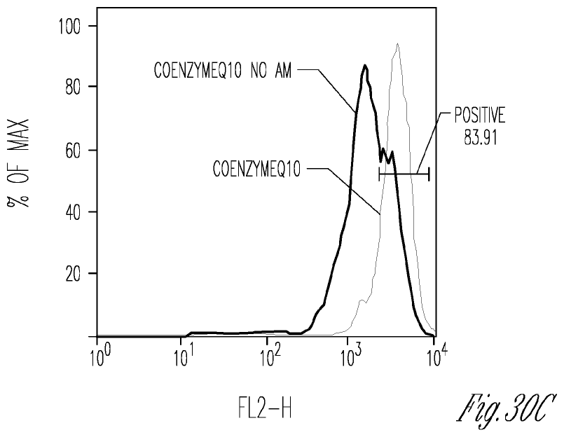

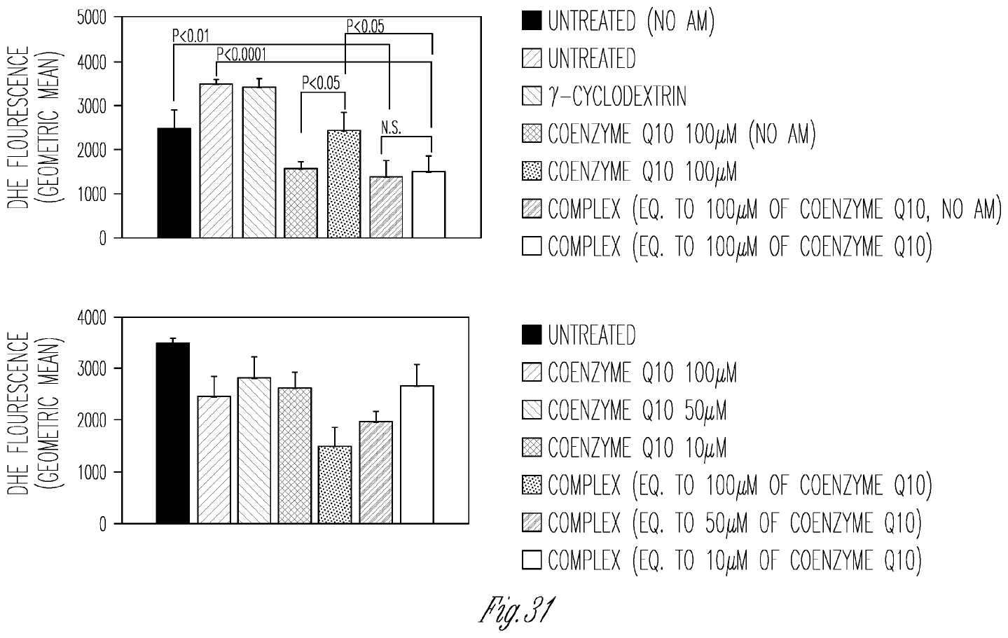

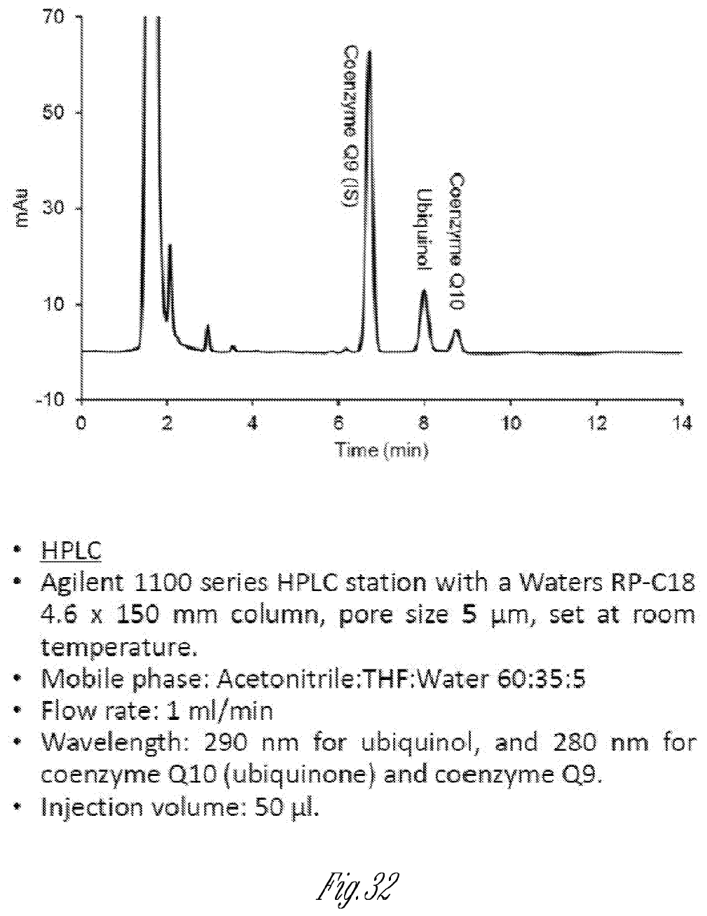

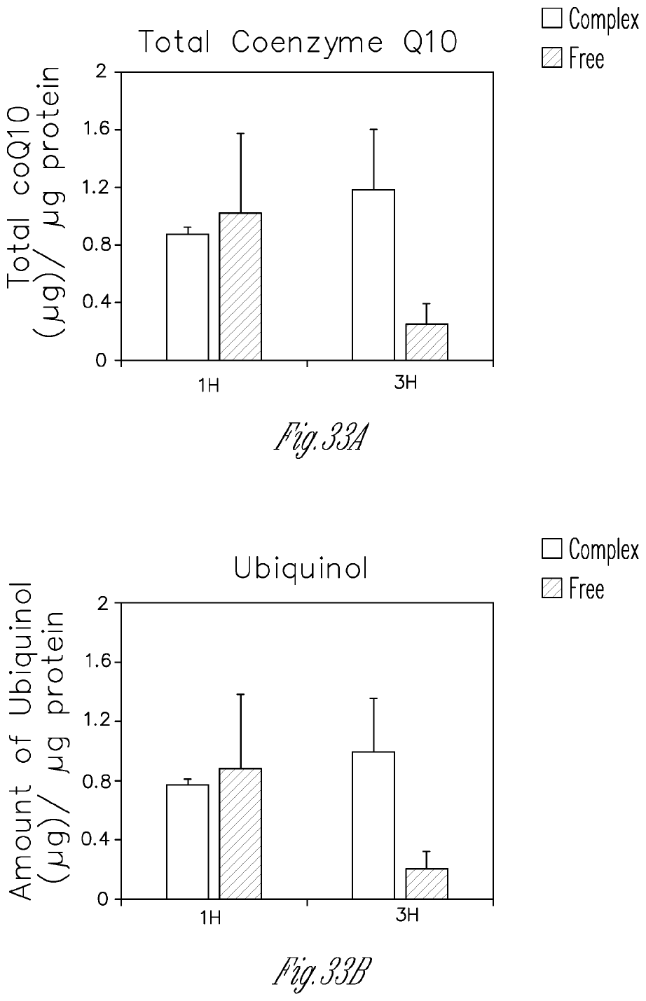

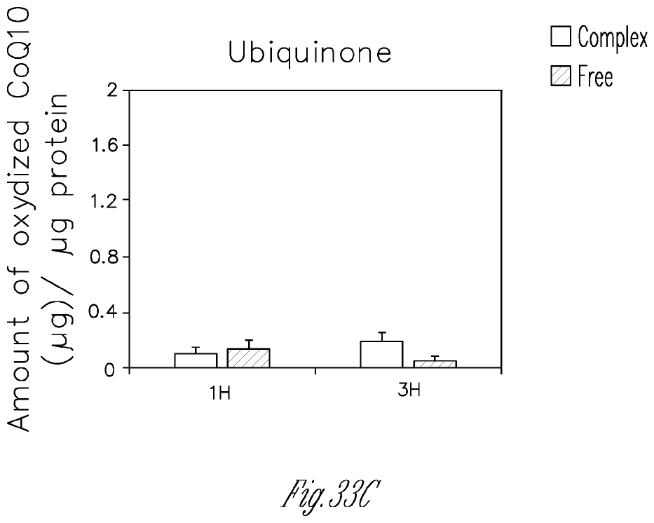

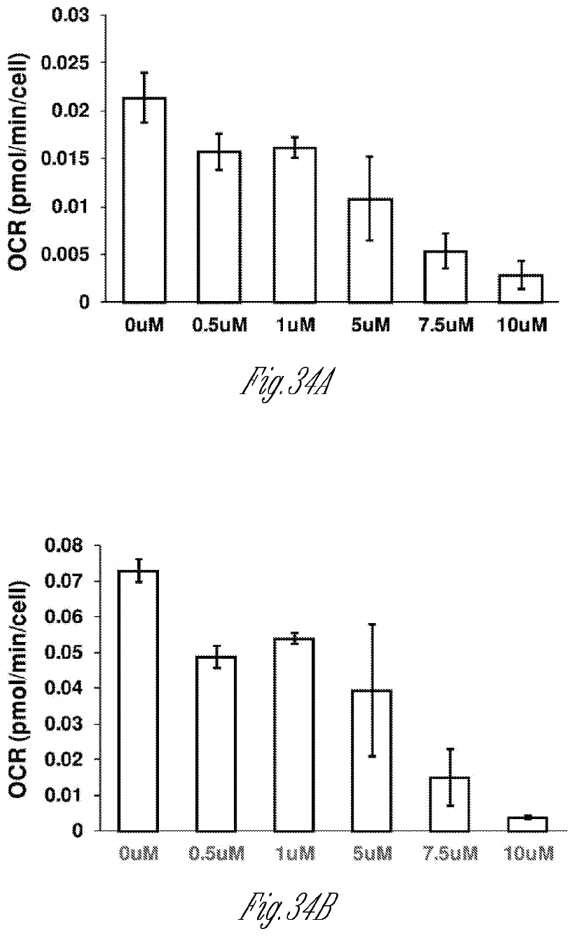

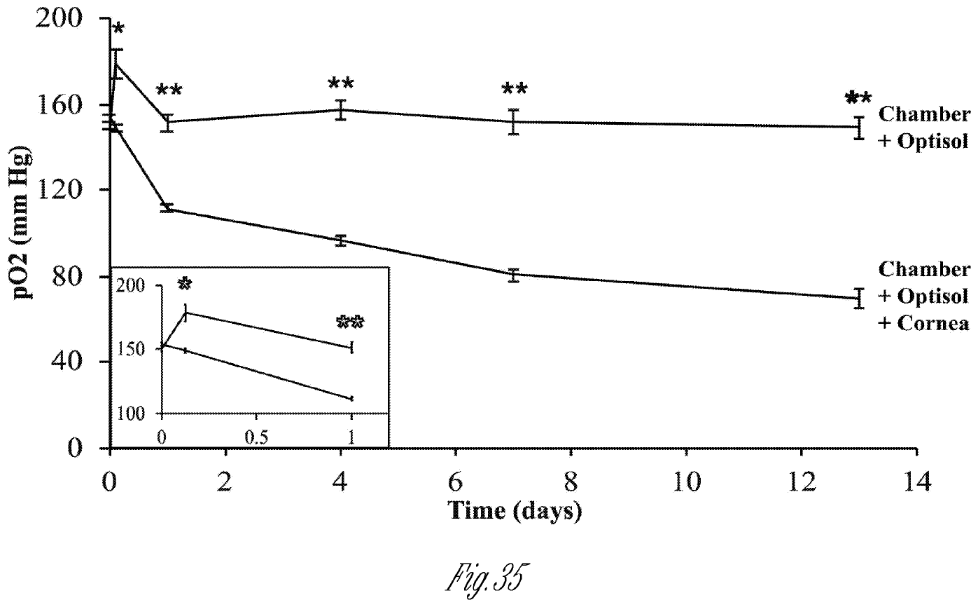

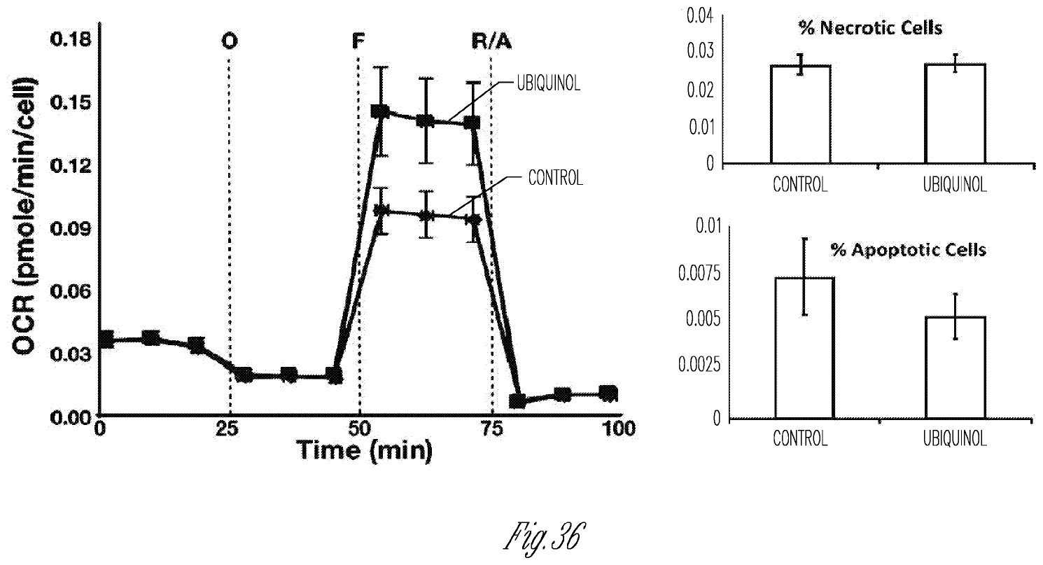

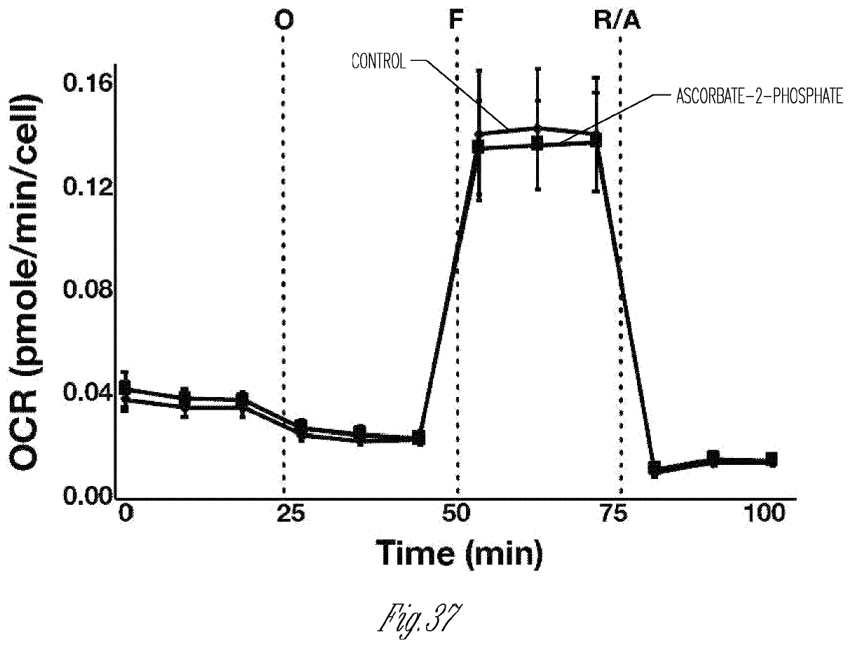

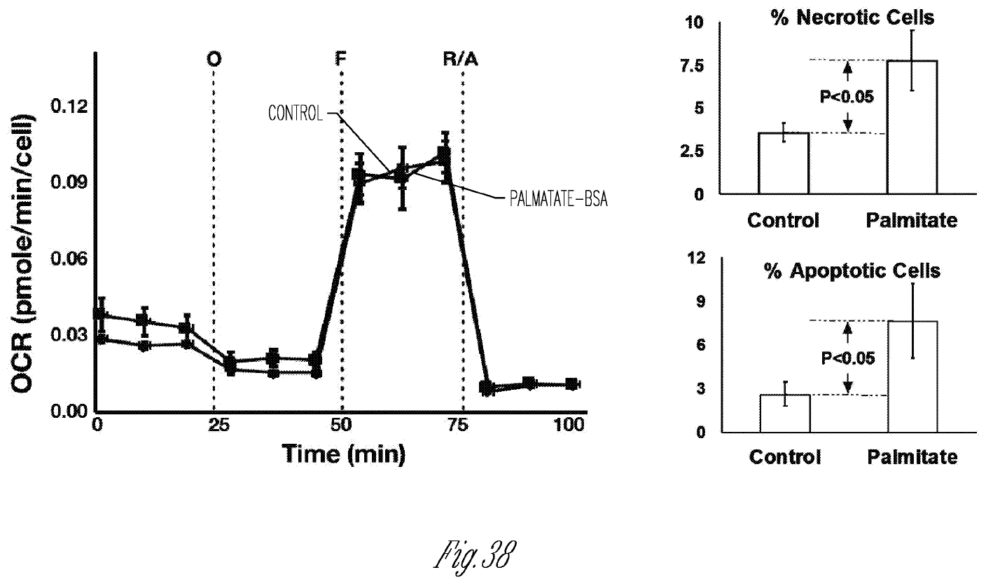

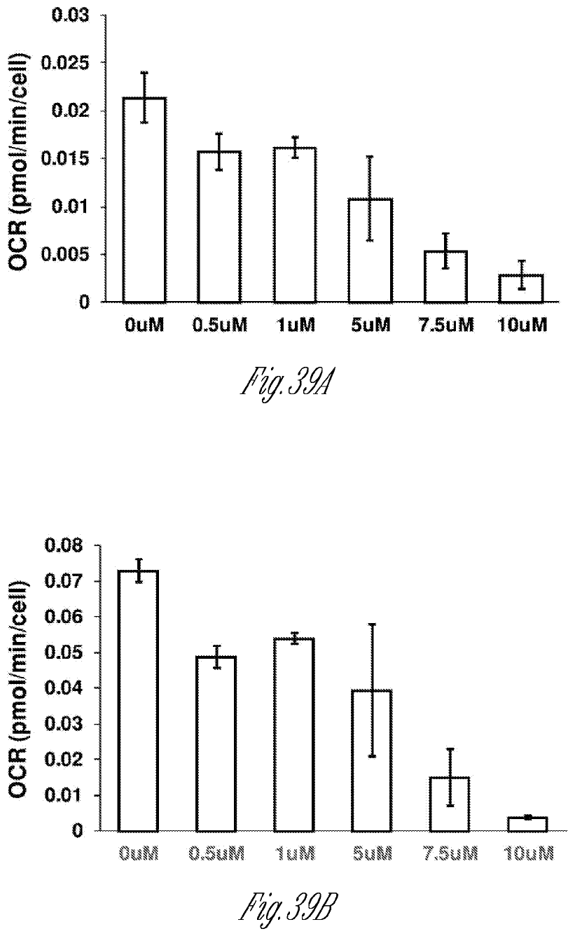

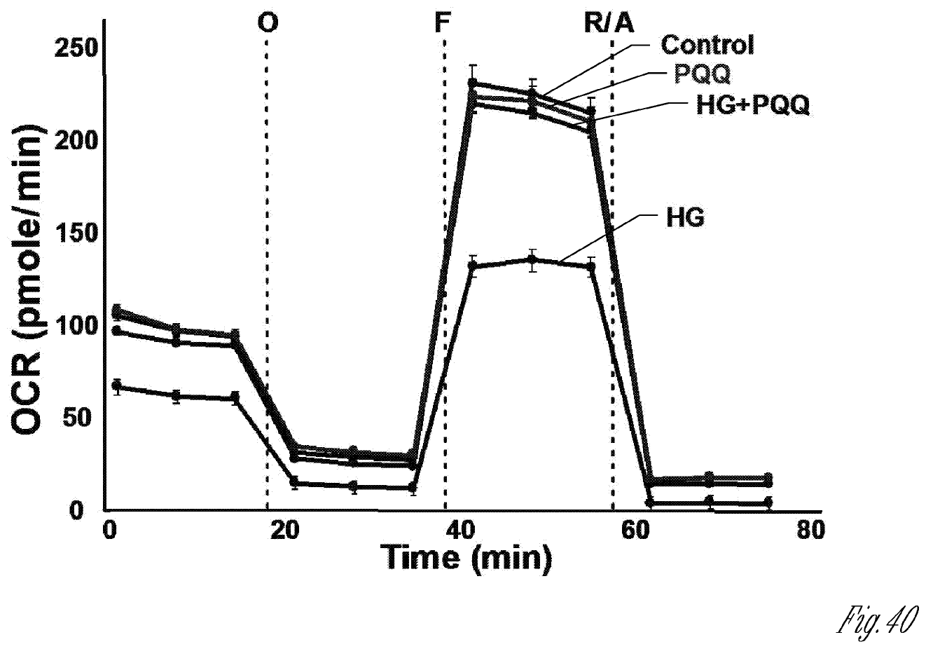

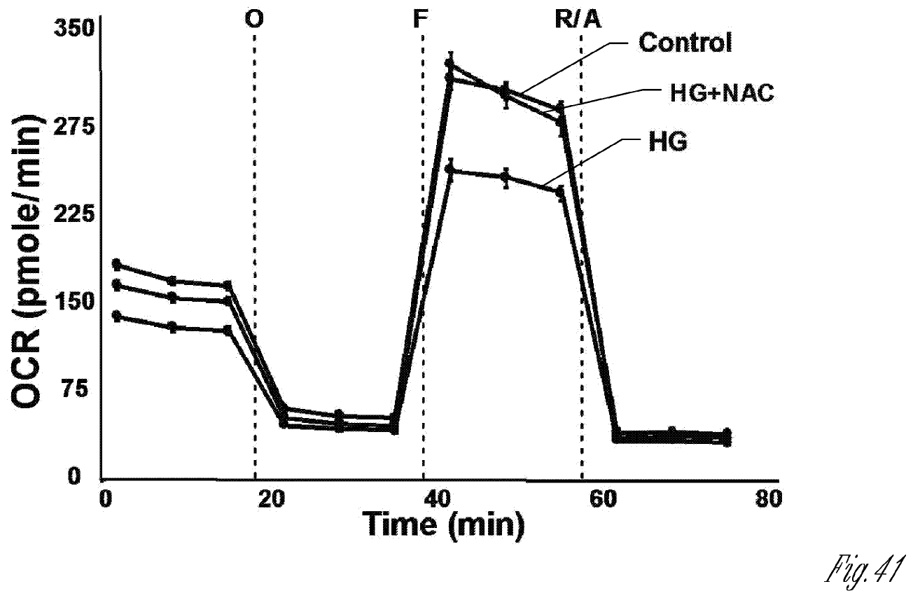

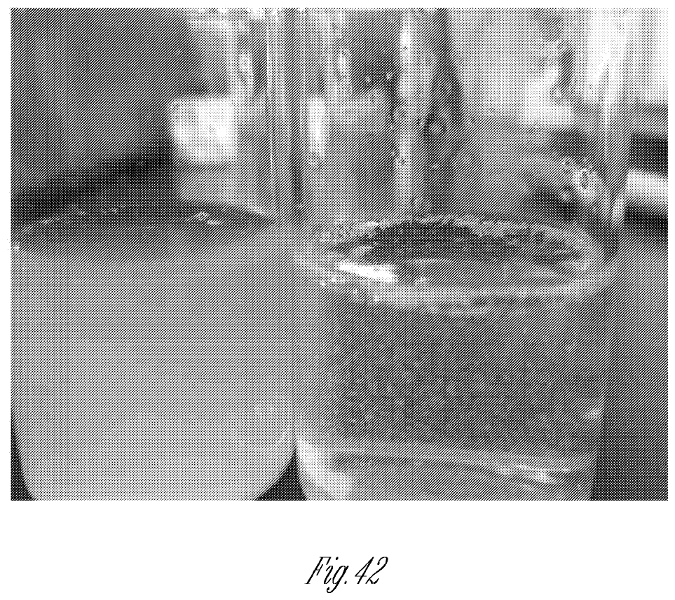

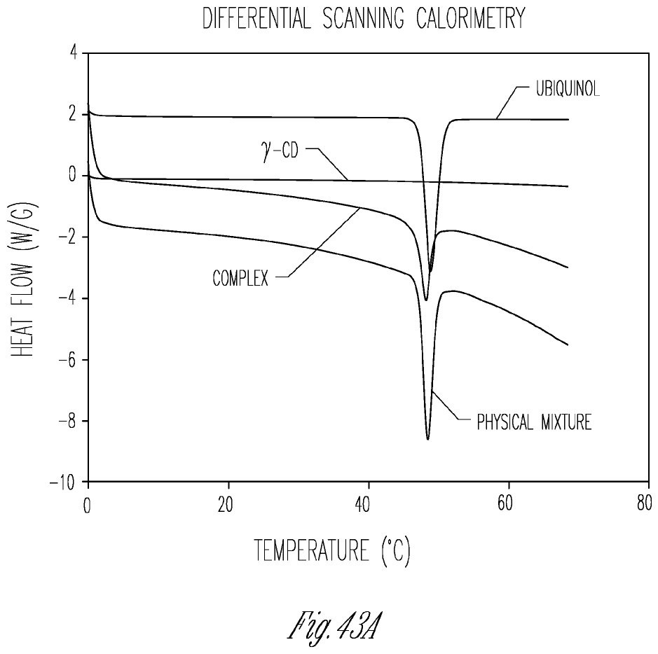

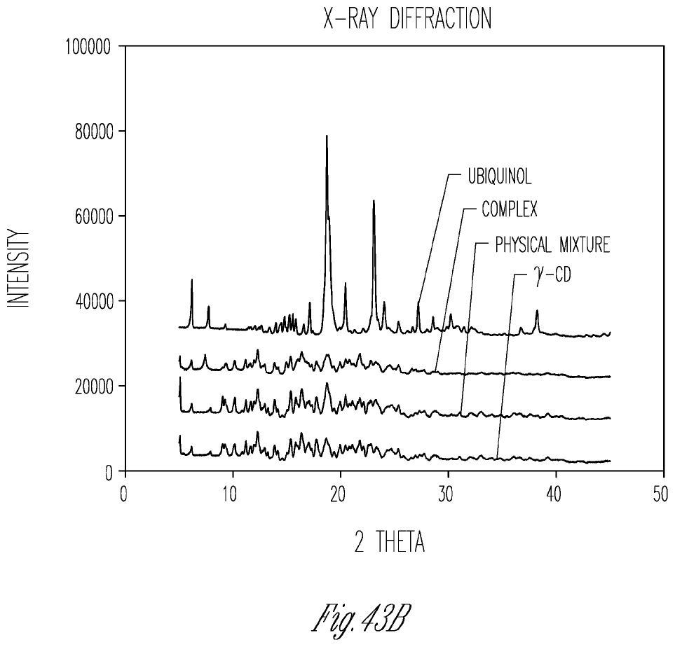

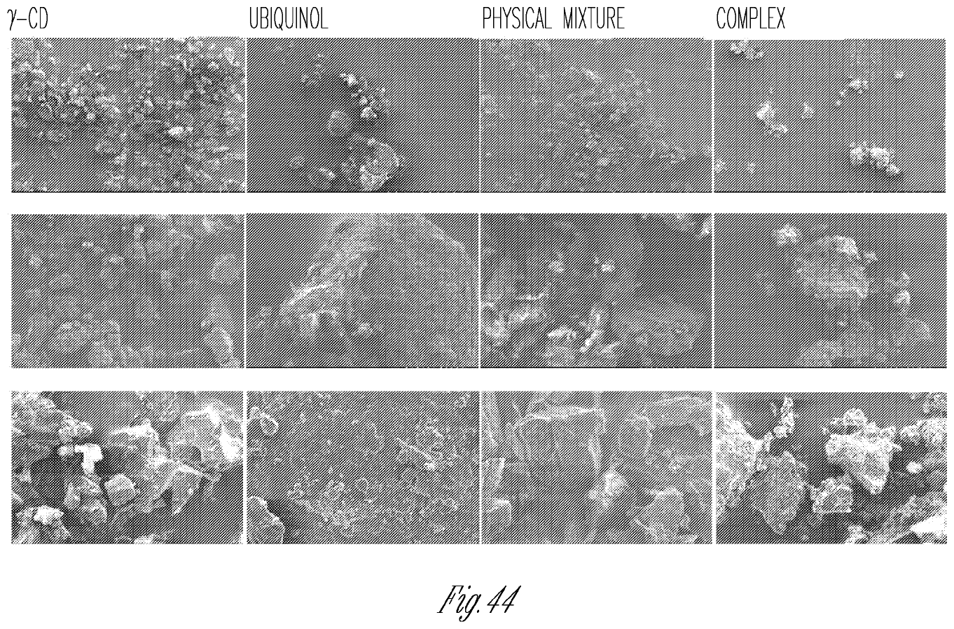

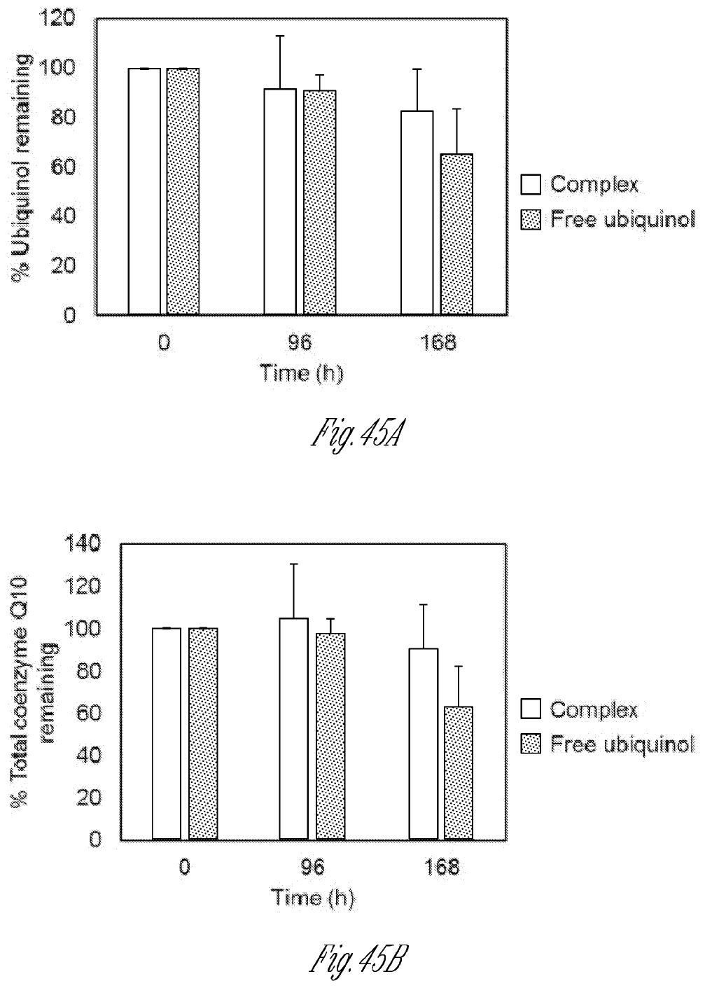

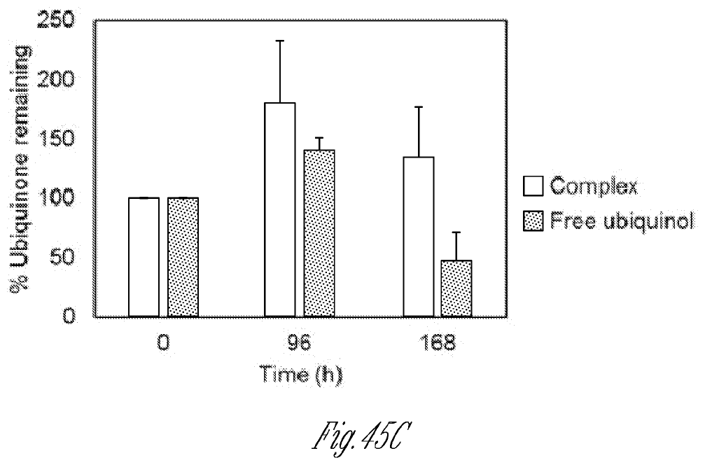

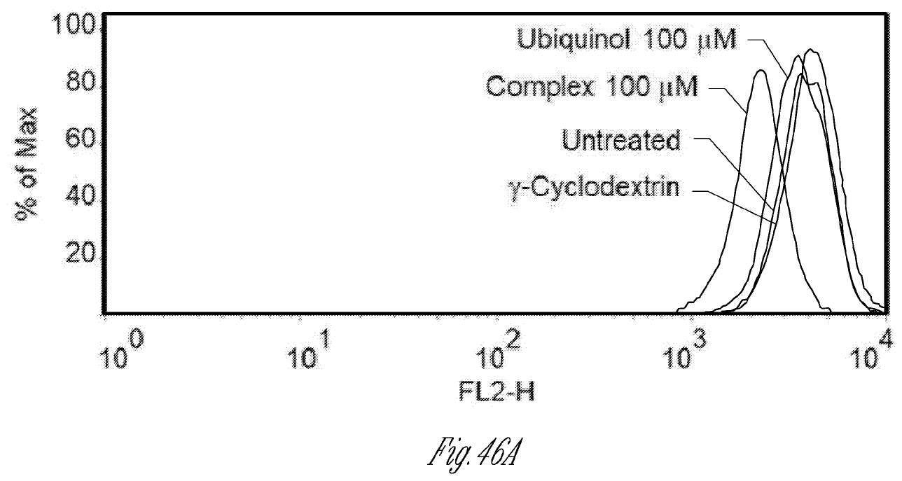

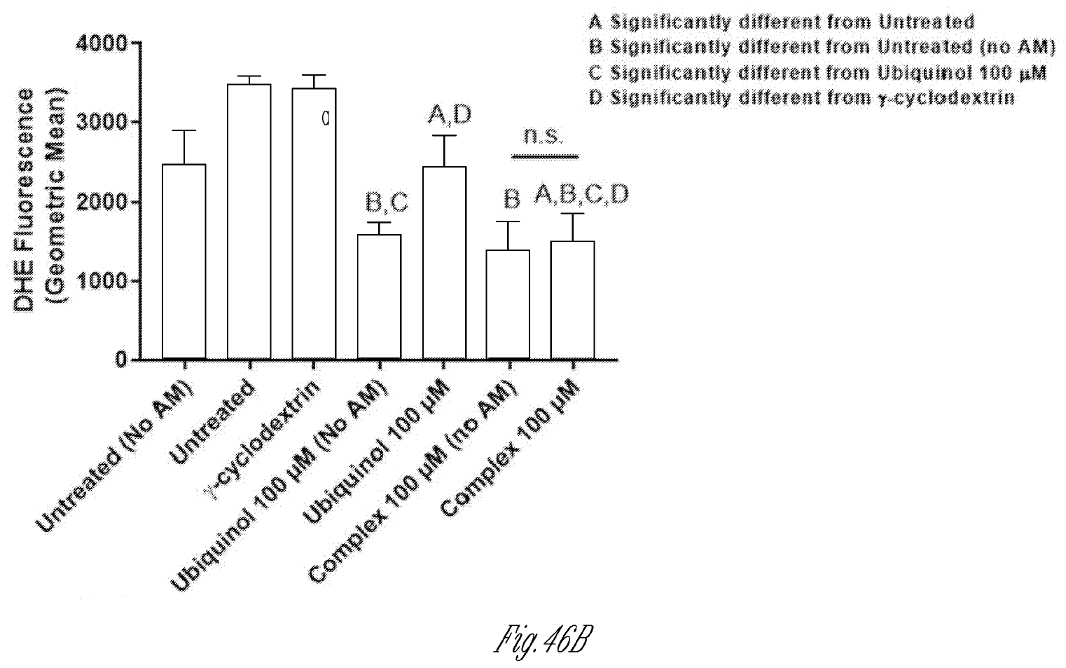

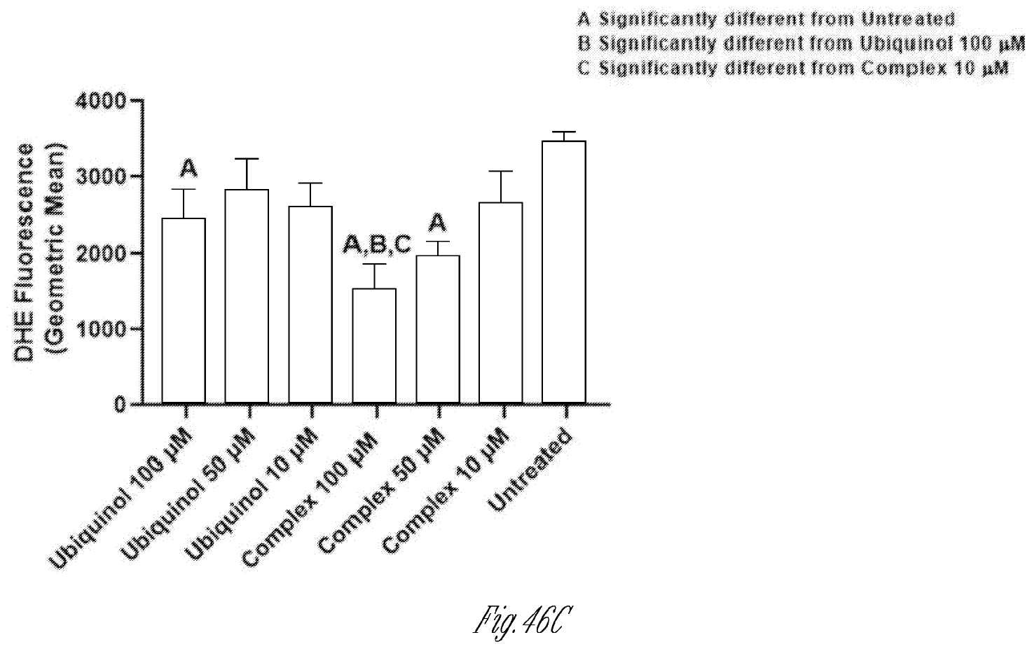

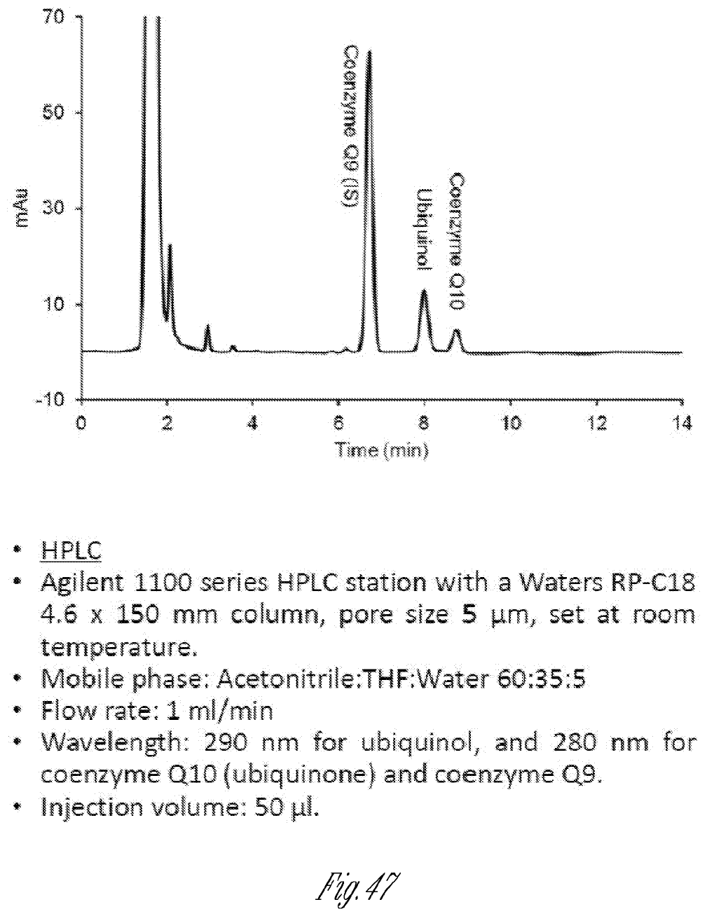

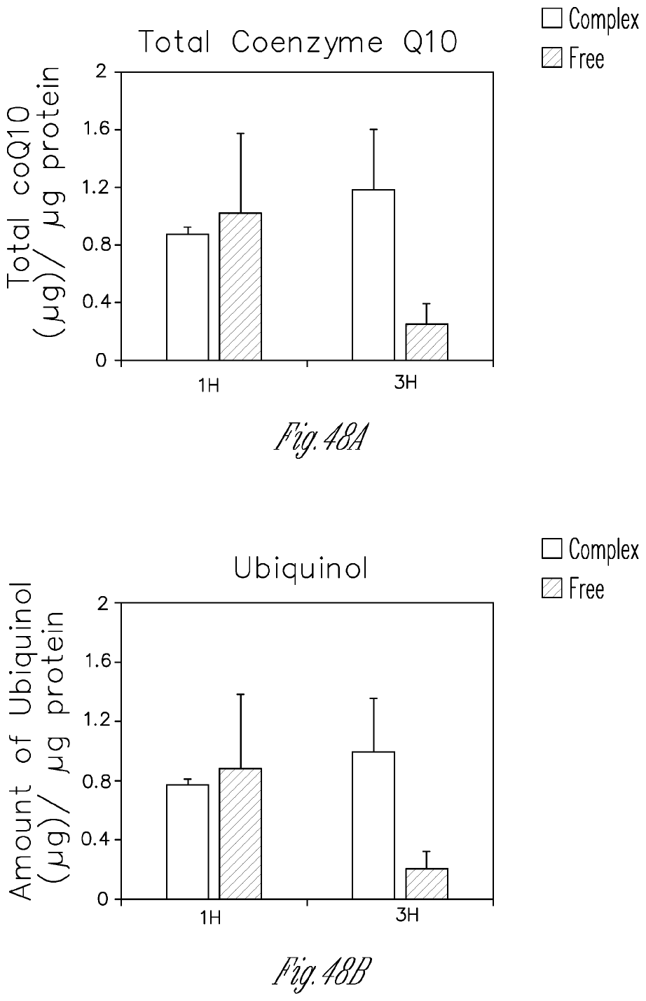

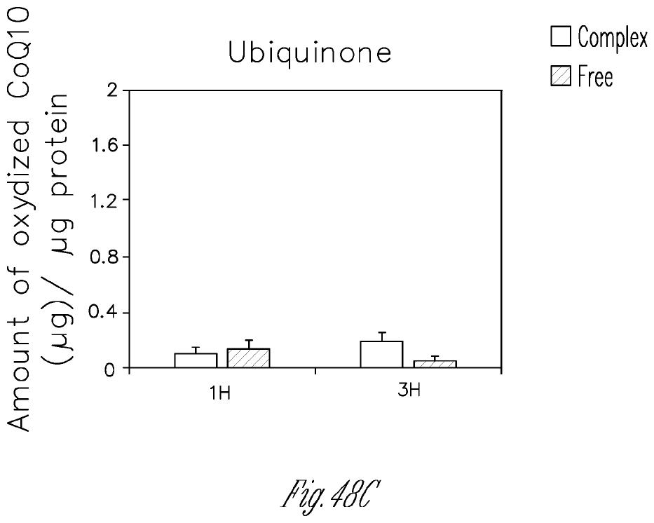

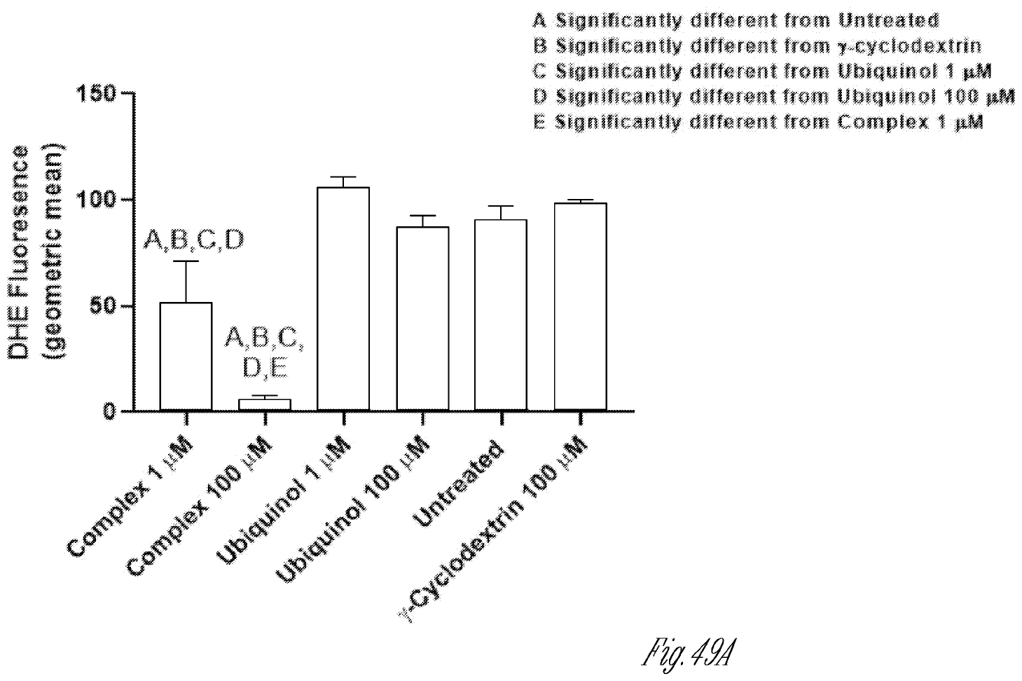

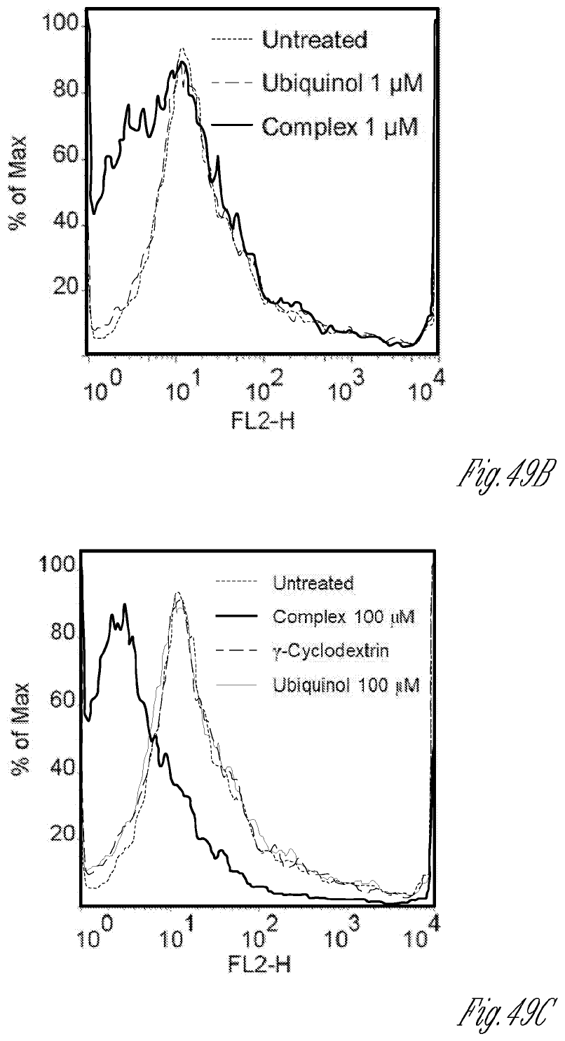

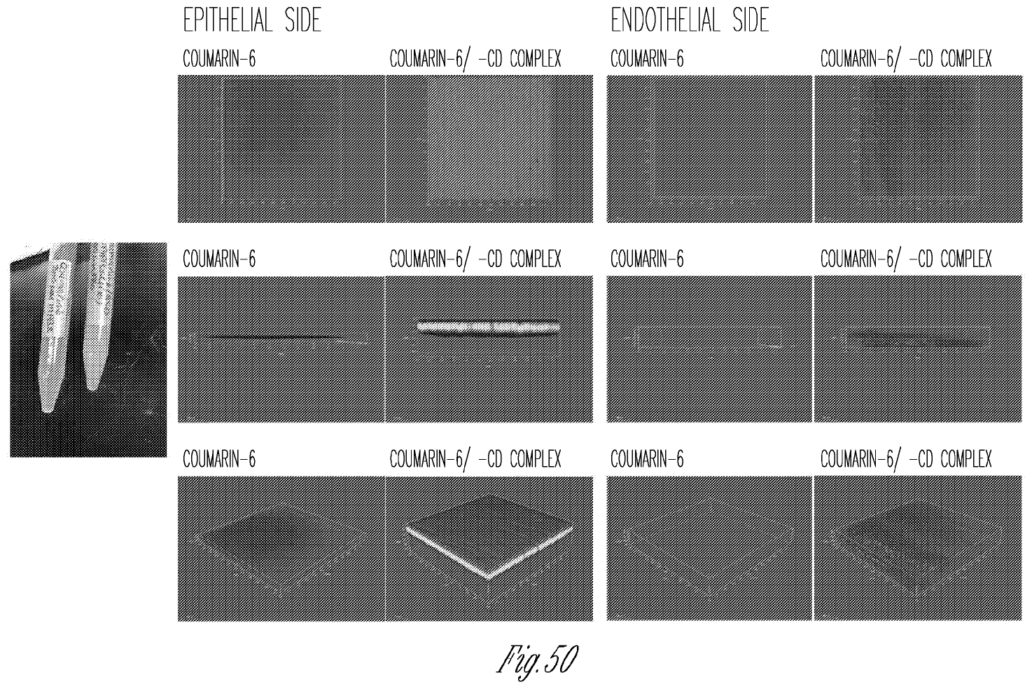

In addition, immortalized human corneal endothelial cells with cyclodextrin-coQ10 were tested or ROS and mitochondrial depolarization using plate reader quantification assays. Two concentrations of the complex were studied, 1 μM and 100 μM. It appears that low concentrations of cyclodextrin-coQ10 reduces mitochondrial ROS, however high doses did not (FIG. 8).

### Conclusions

Complexed coQ10 with cyclodextrin may be employed for different applications including transplant tissue storage medium supplementation, ophthalmic topical drops, and ophthalmic injections.

Coenzyme Q10 is not only a safe addition to cornea storage medium, but it enhances the function of the corneal endothelial cell mitochondria and decreases their overall death. Coenzyme Q10 is a supplement that may enhance transplant tissue and reduce graft failure overall in the future. Also, soluble coQ10 developed for clinical use for ROS affected conditions (diabetes, prior surgeries) in the form of topical drops and injections may reduce the need for transplants in general. Both applications will bolster corneal endothelial cell health by reducing susceptibility to ROS mediated dysfunction, altogether preventing cell loss, vision loss from corneal edema and improving transplant survival.

### Example 7

Ubiquinol is a very potent anti-oxidant, and is the reduced form (more active) of co-enzyme Q10. Due to its lipophilicity and water insolubility, the bioavailability of the drug is very poor. Gamma-cyclodextrin was used to prepare supramolecular inclusion complex with the drug to enhance its wettability and dispersability.

### Solution Method

γ-CD was dissolved in water at different concentrations, and ubiquinol was added to it while stirring (protected from light).
Stirring continues for 2-5 days. Finally, the formed precipitate (complex) was collected and dried.
Yellow discoloration, which indicates the oxidation of ubiquinol following the formation of the yellow oxidized form (ubiquinone), was noticeable at different degrees depending on the stirring time.

### Kneading Method

Ubiquinol and γ-CD (1:10 molar ratio) were mixed under the hood and under light protection, then triturated together using a mortar and pestle by the help of a hydro-alcoholic solution (water:ethanol 1:1) for up to 1 h.
The resulting paste was dried under vacuum for 12-24 h.
The resulting paste was white in color, with no slight yellowish discoloration.

Kneading method was used as the solution method resulted in ubiquinol oxidation in water.

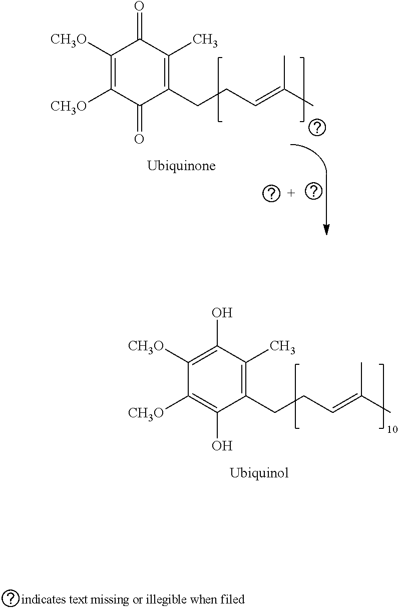

50 mg of kneaded complex added to 10 ml H₂O and shaken for 2 hours.

In FIG. 10, the image on the right is 5 mg of CoQ10 added to 10 ml H₂O and shaken for 2 hours. The complex has high dispersibility in water compared to the free drug. The free drug is very lipophilic and prefers to accumulate at the air-water interface or to adhere to the glass container. In FIG. 10, the image on the left shows 50 mg of kneaded complex added to 10 ml H₂O and shaken for 24 hours.

In FIG. 11, the image on the right is 5 mg of CoQ10 added to 10 ml H₂O and shaken for 24 hours. Even though the complex still retains remarkably higher dispersibility in water compared to the free drug, yellowish discoloration was noticeable in both vials. The instability of ubiquinol in water after both free drug or the complex was stirred in water for 24 h explains why the kneading method that involves the minimum amount of water was chosen form preparation.

Free ubiquinol, gamma cyclodextrin, a physical mixture of the two, and the complex were scanned using differential scanning calorimetry (DSC) and X-ray diffraction (XRD) (FIG. 12). The endothermic peak associated with the melting of ubiquinol exhibited a marked decrease in value and a slight shift towards a lower temperature, compared to free drug or the physical mixture. This may indicate incomplete interaction between the complex and cyclodextrin. This may be preferred because the uncomplexed drug becomes available for immediate anti-oxidant action, compared to the slowly released complexed drug. The indispersibility of the free drug prevents efficient and uniform anti-oxidant activity in aqueous based solutions.

XRD patterns show that the major crystallinity peak of ubiquinol at 2 theta value of 19 is still slightly retained in the physical mixture only.

### ROS Assay

A549 human lung cancer cells were seeded in 6-well plates at 200,000 cells/well for 40 hours, then the medium was removed, and treatments were added. Ubiquinol (coQ10) was dispersed in RPMI medium at three different concentrations (100, 50, and 10 μM) and added to wells (n=3 each), then the volume of each well was completed to 4 ml with medium. Ubiquinol-γ-cyclodextrin (1:10 molar ratio) complex dispersed in RPMI at three different concentrations (equivalent to 100, 50, and 10 μM of ubiquinol) and added to wells (n=3/each) and the volume of the each well was completed to 4 ml with medium._γ-cyclodextrin was added in amounts equivalent to those associated with 100, 50, and 10 μM of the complex to each well (n=3/each) and the volume of the each well was completed to 4 ml with medium._Six wells were left untreated._After 24 h, media were removed, and wells were washed with 5 mM sodium pyruvate in PBS, trypsinized, then collected in 15 ml tubes by centrifugation. Cells were washed with 5 mM sodium pyruvate in PBS, then re-suspended in 1 ml of the same solution._The reagents were added as described in FIG. 14, then incubated at 40 min._Cells were re-suspended and transferred to round-bottom tubes, and analyzed by flow Cytometry. See FIGS. 14-15.

### A549 Cellular Uptake

A549 cells seeded in 6-well plates at 150,000 cells/well. After 48 hours, the cells were treated with 100 μM of ubiquinol either as a complex (1:10 molar ratio of ubiquinol: γ-cyclodextrin) or as free drug. Serial dilutions of ubiquinol were made in RPMI media. After 1 and 3 hours of treatment, 1 ml of cell lysis solution was added to each well (1:1 mixture of 2% w/v SDS and 1% w/v Triton X-100) and the plates were incubated for 15 minutes at 37° C.

Cell lysate was collected and frozen under −80° C. Cell lysate was thawed in ice, then 0.5 ml of cell lysate was spiked with 10 μl of 1 mg/ml solution of coenzyme Q9 in acetonitrile:THF (62:38) as internal standard, and mixed. Two ml of ethyl acetate were added to each sample, and then the sample tube was vortexed for 5 minutes to extract the drug and IS, then centrifuged (21000×g, 5 min). The organic layer was separated in a glass tube. The extraction was repeated one more time. Four ml of ethyl acetate was then evaporated under nitrogen, then the residue was reconstituted in 87.5 μl of THF. It was centrifuged, then the supernatant was diluted with acetonitrile and water at a ratio of THF:acetonitrile:water of 35:60:5. The samples were injected into the HPLC for analysis.

### Example 8

Seahorse respiration assays were conducted with MitoQ with doses ranging from 0 to 10 μM. All experiments were performed on corneal endothelial cells in culture and analyzed using the XFe96 Extracellular Flux Analyzer.

Unlike the control formulation, there was a clear negative influence of the MitoQ formulation on mitochondrial respiration activity of cultured corneal endothelial cells. Specifically, a dose dependent decrease in ATP linked oxygen consumption that was significantly different between the control and highest dose tested was observed (P<0.001; FIG. 19A). Likewise, a similar dose dependent decrease in spare respiratory capacity that was significantly different between the control group and the highest dose tested was observed (P<0.001; FIG. 19B). Similar dose dependent reductions were observed for basal respiration and maximal respiration (data not shown) but proton leak and non-mitochondrial respiration were not influenced by MitoQ treatment (data not shown).

In summary, a dose dependent decrease in respiratory function was observed with MitoQ (FIG. 19).

In one embodiment, complexes may be prepared by kneading under conditions that include low light, low moisture, low oxygen, or a combination thereof, using a molar ration of 1:10 (anti-oxidant to carrier such as ubiquinol:gamma-cyclodextrin) which is kneaded in the presence of ethanol and water (e.g., 1:1) using a mortar, e.g., porcelain mortar, in the hood for about 45-60 minutes, then drying the kneaded mixture under vacuum, e.g., in a dessicator, for about 6 to 8 hours The product may be stored at −20° C., e.g., in amber Eppendorf tubes.

### Example 9

To determine if adding the anti-oxidant Coenzyme Q10 (CoQ10 or ubiquinol) to donor cornea storage media enhances the metabolic function of corneal endothelial cells (CECs) and/or decreases overall cell death in storage the following experiments were conducted.

### Methods

Human corneal tissue pairs were obtained by Iowa Lions Eye Bank (ILEB) from nondiabetic donors 60-75 years old and stored in Optisol GS (Bausch+Lomb, Irvine, Calif.) at 4° C. following procurement in accordance with Eye Bank Association of America and ILEB policies and procedures. For 5 days prior to testing, but within 9 days of procurement, one stored tissue from a corneal pair was treated with 10 μM CoQ10, while the mate tissue was treated with diluent only as a control. Descemet membrane and endothelial cell punches were collected and mounted onto the bottom of a Seahorse assay plate (cells facing upward). Mitochondrial respiration was assayed by measuring oxygen consumption using the Seahorse XFe24 Extracellular Flux Analyzer (Agilent Technologies, Santa Clara, Calif.) over 120 minutes (9-minute intervals). Punches were labeled with a nuclear counterstain (DAPI) and remaining tissues were mounted onto slides and labeled with 488A-Annexin V, Ethidium Homodimer III, and Hoechst 33342 (Apoptotic, Necrotic, and Healthy Cells Quantification Kit; Biotium, Fremont, Calif.) to assay cell health. Nuclei were counted in each punch to normalize respirometry data. Immunohistochemistry densitometry was measured using ImageJ software.

Next, the effects of lower doses (0.5, 1, 5, and 7.5 μM) on tissues in storage were examined using the same methods.

### Results

In total, 14 paired corneas were tested. Three different aspects of mitochondrial respiration were affected by CoQ10 treatment: proton leak was increased 34% (p=0.046), maximal respiration was increased 97% (p=0.003), and spare respiratory capacity was increased 133% (p<0.001). Corneal endothelial cell necrosis was not changed, however, apoptosis was reduced 29% in treated cells (p=0.09). Please refer to FIG. 20.

At lower doses, it was found that 1 μM protected not only against apoptosis, but also against necrosis. This concentration did not show a bolstering of mitochondrial spare respiratory capacity function as seen with the 10 μM dose. It did show the largest increase in non-mitochondrial respiration. Also, there was no increase in proton leak at 1 μM. This is a positive finding since proton leak may be an indicator of early depolarization.

### Conclusions

In this series, Coenzyme Q10 increased corneal endothelial cell mitochondrial respiration and prevented cells from dying in storage. Findings indicate that Optisol GS supplemented with CoQ10 may reduce presurgical cell death and functional decline related to tissue storage. Further studies determine the dosing strategy during storage as well as the cytoprotective effects on cell density after endothelial keratoplasty. At this point, it is indicated that coQ10 supplementation may provide two protective effects, at different concentrations. At the lower concentration, it appears to protect overall cell health, decreasing both apoptosis and necrosis, but not alter mitochondrial respiration. At the higher dose, apoptosis is reduced and mitochondrial spare capacity is bolstered. However, also at the higher dose, proton leak increases.

### Example 10

To determine if adding ascorbate-2-phosphate to donor cornea storage media enhances the metabolic function of corneal endothelial cells (CECs) the following experiments were conducted.

### Methods

Human donor whole globe eye pairs were obtained by Iowa Lions Eye Bank (ILEB) and the anterior portion of the eyes were removed. For 14 days, one cornea was stored from a corneal pair treated with 1 mM ascorbate-2-phosphate, while the mate tissue was treated with diluent only as a control. Descemet membrane and endothelial cell punches were collected and mounted onto the bottom of a Seahorse assay plate (cells facing upward). Mitochondrial respiration was assayed by measuring oxygen consumption using the Seahorse XFe24 Extracellular Flux Analyzer (Agilent Technologies, Santa Clara, Calif.) over 120 minutes (9-minute intervals). Punches were labeled with a nuclear counterstain (DAPI) and nuclei were counted in each punch to normalize respirometry data.

### Results

In total, 5 paired corneas were tested. The only change found was in non-mitochondrial respiration, with treated cells having a 21% higher level than untreated control cells (p-value=0.053). Please refer to FIG. 21.

### Conclusions

In this series, ascorbate-2-phosphate did not affect corneal endothelial cell mitochondrial respiration at the dose (1 mM) used. This indicates that ascorbate-2-phosphate is safe for use in corneal endothelial cell storage.

### Example 11

To determine if adding palmitate-BSA to donor cornea storage media enhances the metabolic function of corneal endothelial cells (CECs) and/or decreases overall cell death in storage the following experiments were conducted.

### Methods

Human corneal tissue pairs were obtained by Iowa Lions Eye Bank (ILEB) from nondiabetic donors 60-75 years old and stored in Optisol GS (Bausch+Lomb, Irvine, Calif.) at 4° C. following procurement in accordance with Eye Bank Association of America and ILEB policies and procedures. For 5 days prior to testing, but within 9 days of procurement, one stored tissue from a corneal pair was treated with 100 μM palmitate-BSA, while the mate tissue was treated with BSA only as a control. Descemet membrane and endothelial cell punches were collected and mounted onto the bottom of a Seahorse assay plate (cells facing upward). Mitochondrial respiration was assayed by measuring oxygen consumption using the Seahorse XFe24 Extracellular Flux Analyzer (Agilent Technologies, Santa Clara, Calif.) over 120 minutes (9-minute intervals). Punches were labeled with a nuclear counterstain (DAPI) and remaining tissues were mounted onto slides and labeled with 488A-Annexin V, Ethidium Homodimer III, and Hoechst 33342 (Apoptotic, Necrotic, and Healthy Cells Quantification Kit; Biotium, Fremont, Calif.) to assay cell health. Nuclei were counted in each punch to normalize respirometry data. Immunohistochemistry densitometry was measured using ImageJ software.

### Results

In total, 10 paired corneas were tested. The only significant respiration change found was in proton leak, with treated cells having a 45% higher proton leak than untreated control cells (p-value=0.031). Looking at the overall levels of apoptosis and necrosis however, it was found that the treated cells also had a 116% higher amount of necrosis (p-value=0.0353) and 67% higher amount of apoptosis (p-value=0.0286). Please refer to FIG. 22.

### Conclusions

In this series, palmitate-BSA did not enhance corneal endothelial cell mitochondrial respiration or prevent cells from dying in storage. On the contrary, palmitate-BSA increased apoptosis, necrosis, and proton leak and therefore may actually be toxic to the cells at the dose tested.

### Example 12

To determine if adding mitochondria specific coenzyme Q10 (MitoQ) to donor cornea storage media enhances the metabolic function of corneal endothelial cells (CECs) the following experiments were conducted.

### Methods

Human corneas were obtained by Iowa Lions Eye Bank (ILEB) from nondiabetic donors 60-75 years old and stored in Optisol GS (Bausch+Lomb, Irvine, Calif.) at 4° C. following procurement in accordance with Eye Bank Association of America and ILEB policies and procedures. Endothelial cells were isolated and cultured in Seahorse XFe96 well plates until they reached confluency. Purity of cell cultures were confirmed with anti-zonula occludens 1 (ZO-1) labeling of cellular tight junctions. Once confluent, cells were treated with MitoQ with doses ranging from 0 to 10 μM. Mitochondrial respiration was assayed by measuring oxygen consumption using the Seahorse XFe96 Extracellular Flux Analyzer (Agilent Technologies, Santa Clara, Calif.) over 120 minutes (9-minute intervals). Wells were labeled with a nuclear counterstain (DAPI) and nuclei were counted in each well to normalize respirometry data.

Unlike the control formulation, there was a clear negative influence of the MitoQ formulation on mitochondrial respiration activity of cultured corneal endothelial cells. Specifically, a dose dependent decrease in ATP linked oxygen consumption that was significantly different between the control and highest dose tested was observed (P<0.001; FIG. 34 left). Likewise, a similar dose dependent decrease in spare respiratory capacity that was significantly different between the control group and the highest dose tested was observed (P<0.001; FIG. 34 right). Similar dose dependent reductions were observed for basal respiration and maximal respiration (data not shown) but proton leak and non-mitochondrial respiration were not influenced by MitoQ treatment (data not shown).

### Conclusions

Escalating the dosage of MitoQ resulted decreased in respiratory function. It is therefore indicated that high doses of MitoQ do not protect corneal endothelial cells as well as ubiquinol and that further research is needed on dosage strategies and effects on cellular responses other than respiratory function to determine if there is any protective effect.

### Example 13

### Summary

To determine whether ubiquinol or palmitate improves mitochondrial function and cell viability in human donor corneal endothelial cells (CECs) during hypothermic cornea tissue storage. Endothelial cell-Descemet membrane (EDM) tissues were treated with 10 μM ubiquinol, the reduced form of the antioxidant coenzyme Q10, or 100 μM palmitate conjugated with bovine serum albumin (BSA), a fatty acid used in antioxidant formulations and as a preservative, for 5 days in Optisol-GS storage media prior to assaying for mitochondrial activity using extracellular flux analysis of oxygen consumption. Additionally, EDM tissues were analyzed for cell viability using apoptosis and necrosis assays. Control tissues from mate corneas were treated with diluent only and comparisons were analyzed for differences.

Ubiquinol treatment (N=13) increased spare respiratory capacity 174% (p=0.001), maximal respiration 93% (p=0.003), and proton leak 80% (p=0.047) compared to controls. In contrast, palmitate-BSA treatment (N=7) only increased proton leak by 64% (p=0.045) compared to controls. Cells treated with ubiquinol had no significant change in cell necrosis or apoptosis, but cells treated with palmitate-BSA had a 90% increase in necrosis (p=0.024) and 200% increase in apoptosis (p=0.028), indicating cytotoxicity.

Thus, ubiquinol may be an useful biocompatible additive to hypothermic corneal storage media that increases CEC mitochondrial function, whereas palmitate-BSA reduces CEC viability. Additional investigations are indicated to further investigate and optimize the dose and formulation of ubiquinol for use in preserving donor corneal tissue function during hypothermic storage.

### Introduction

In this study, the effects of supplementing hypothermic corneal storage media with ubiquinol—the reduced and active form of coenzyme Q10 (CoQ10) that lowers intracellular ROS and helps establish the proton force required for oxidative phosphorylation and ATP synthesis (Diaz-Casado et al., 2019; Ebadi et al., 2001;

Hirst et al., 2016; Mellors et al., 1966) were evaluated to determine the relative effects on mitochondrial respiration and cell viability compared to controls. The performance of ubiquinol was compared against diluent agent alone in the mate cornea to minimize physiologic variability. In addition, the performance of palmitate-BSA—a fatty acid metabolite that has been shown to increase mitochondrial spare respiratory capacity and reduce cell death following hypoxia in high energy demanding cardiac myocytes (Pfleger et al., 2015) was tested against BSA-only controls in similarly paired donor corneas in order to assess its effects on cell function compared to ubiquinol. This study determined whether supplementation of cold storage media with an agent that augments mitochondrial function may be a viable strategy in preventing donor tissue cell loss, particularly as the demand for donor corneal tissue continues to grow worldwide.

### Materials and Methods

All experimental procedures conformed to the Declaration of Helsinki. The Institutional Review Board at the University of Iowa has determined that approval was not required for this study and research consent was obtained for all donor corneas.

### Donor Corneas:

Corneoscleral tissues were obtained, inspected, and stored in Optisol-GS (Bausch+Lomb) at 4° C. in accordance with Eye Bank Association of America and Iowa Lions Eye Bank (ILEB) compliant protocols. All tissues were deemed suitable for cornea transplantation according to standard ILEB protocols, and all experimental testing was performed within 14 days of procurement. Prior to assays, tissues were analyzed via non-contact specular microscopy (KeratoAnalyzer EKA-10; Konan Medical USA, Irvine, Calif.) to quantify endothelial cell density (ECD), hexagonality (hex), and coefficient of variation (CV) from the average of 3 independent images obtained using a 50 cell, center count method. All tissues were also examined by slit lamp (BQ-900 LED; Haag-Streit Diagnostics, Mason, Ohio) to assess for tissue health according to standard protocols at ILEB. Corneas were excluded from testing if the review of medical records or postmortem serology results conducted by ILEB technicians revealed evidence of sepsis or infectious disease. Donor tissue characteristics collected for this study were donor age, ECD, CV, hex, death to preservation time (D/P), and preservation time to assay (P/A).

### Experimental Groups and Reagents:

Paired corneas were supplemented with 10 μM ubiquinol (Fuller et al., 2006) (USP analytical standard, Sigma Aldrich, St. Louis, Mo.), 100 μM palmitate-BSA (Pfleger et al., 2015) (Agilent, Santa Clara, Calif.), or diluent only for 5 days, such that one cornea from a donor received treatment while its mate from the same donor was a control. Concentrations were chosen based on previously published studies (Fuller et al., 2006; Pfleger et al., 2015). Diluents were chosen based on supplement solubility. Although several diluents were tried for ubiquinol (water, Optisol-GS, DMSO and ethanol), ethanol was chosen based on its ability to solubilize ubiquinol most successfully. No complexing agents were used to solubilize ubiquinol. Following the 5 days of storage with supplementation, tissues were processed for metabolic and cell viability assays as described below.

### Metabolic Assays:

Tissue preparation and extracellular flux assays for mitochondrial respiration were performed as described in Greiner et al. (2015). In brief, after pre-stripping the EDM, 3 mm diameter EDM punches were mounted in wells of a XF24 microplate (Agilent Technologies, Santa Clara, Calif.). After acclimation for one hour at 37° C. in non-buffered assay media, metabolic activity of the EDM samples was quantified using a commercial kit (XF Cell Mito Stress Test Kits; Agilent Technologies) on a Seahorse XFe24 extracellular flux analyzer (Agilent Technologies). Following extracellular flux analysis, tissues were labeled fluorescently using a 1:1000 Sytox Green nucleic acid stain in the microtiter plate (Life Technologies, Grand Island, N.Y.) and imaged on an Olympus IX-81 inverted microscope (Olympus America, Center Valley, Pa.) using a FITC filter. Cell counts were determined using Image J (https://imagej.nih.gov/ij/download.html) and used to compute the oxygen consumption rate per cell (OCR; pmole/min/cell). Raw OCR values were used to calculate several different key parameters of metabolic function per manufacturer's directions (Agilent Technologies), as in Aldrich et al. (2017). The parameters and calculations used in this study were the main outcome measures, including basal respiration, ATP-associated oxygen consumption, proton [H⁺] leak, maximal respiration, spare respiratory capacity, non-mitochondrial respiration, and coupling efficiency as described in Schneider et al. and Goldstein et al. (2018).

### Apoptosis and Necrosis Assays:

In brief, excess EDM tissues from surrounding the punches used for metabolic assays were incubated with 488A-Annexin V, Ethidium Homodimer III, and Hoechst 33342 (Apoptotic, Necrotic, and Healthy Cells Quantification Kit; Biotium, Fremont, Calif.) to detect the apoptotic, necrotic, and entire cell populations, respectively. Tissues were imaged on an Olympus IX-81 inverted microscope (Olympus America) and analyzed using Image J to calculate the percent apoptotic, necrotic, and viable cells for each sample.

### Statistical Analysis:

Treatment mean differences in the mitochondrial respiration parameters were compared using linear mixed model analysis for a randomized block design with post-hoc pairwise comparisons using a Tukey-Kramer test. Paired t-tests were used to test for differences in necrosis and apoptosis between treated and control tissues. Statistical significance was defined as p<0.05.

For the mitochondrial respiration assays, a linear mixed model analysis for a randomized block design with Tukey-Kramer post-hoc pairwise comparisons at the 0.05 significance level, assuming a correlation of r=0.50 between pairs, will be able to detect with 0.80 power an effect size of at least 0.90 standard deviations (SD) in pairwise treatment mean differences. For the apoptosis and necrosis assays, a paired t-test at the 0.05 significance level, assuming a correlation of r=0.50 between pairs, will be able to detect with 0.80 power an effect size of at least 0.66 SD.

### Results

7 different components of mitochondrial related respiratory events were analyzed (basal respiration, ATP production, proton leak, maximal respiration, spare respiratory capacity, non-mitochondrial respiration, and coupling efficiency) in transplant suitable donor EDM tissue punches, measured as the oxygen consumption rate and normalized to the cell density of each corneal endothelial tissue assayed. Assays to assess overall levels of apoptosis and necrosis were also performed. Characteristics of donor tissues used in all assays are summarized in Table 1.

TABLE 1
Donor characteristics of comeal tissue by experimental assay.

| | Mean (SEM) | | | |
|---|---|---|---|---|
| | Mitochondrial | | Apoptosis and Necrosis | |
| | Stress Test | | Assay | |
| | | Palmitate- | | Palmitate- |
| | Ubiquinol | BSA | Ubiquinol | BSA |
| Donor Age (years) | 64.2 (2.5) | 67.4 (1.7) | 63.3 (3.3) | 67.3 (1.6) |
| Death to Preservation | 14.3 (1.7) | 9.9 (1.4) | 13.9 (2.2) | 10.2 (1.1) |
| Time (hours) | | | | |
| Preservation Time to | 11.8 (0.4) | 13.4 (0.6) | 11.7 (0.4) | 13.0 (0.6) |
| Assay (days) | | | | |
| ECD (cells/mm²) | 2347.0 | 2651.0 | 2440.3 | 2528.9 |
| | (89.0) | (164.6) | (109.5) | (147.8) |
| Hexagonality (percent)^{#} | 55.4 (1.8) | 55.5 (2.7) | 55.0 (1.2) | 55.9 (1.8) |
| Coefficient of Variation^{#} | 34.3 (1.0) | 33.8 (1.5) | 32.5 (1.7) | 31.9 (0.6) |
| Cornea Pairs (n) | 13 | 7 | 9 | 8 |

^{#}Calculated for a subset of donors due to availability of data (8 of 13 for ubiquinol mitochondrial stress test, 4 of 7 donors for palmitate-BSA mitochondrial stress test, 3 of 9 donors for ubiquinol apoptosis and necrosis assay, and 4 of 8 donors for palmitate-BSA apoptosis and necrosis assay).

### Mitochondrial Respiration:

First, the effects of ubiquinol supplementation were analyzed. 13 paired corneas, one cornea treated with ubiquinol and the mate cornea treated with diluent only as a control, for 5 days, were tested. Three different aspects of mitochondrial respiration were affected by treatment (Table 2, FIG. 36): spare respiratory capacity increased 174% (p=0.001), maximal respiration increased 93% (p=0.003), and proton leak increased 80% (p=0.047) compared to controls. Next, the effects of palmitate-BSA supplementation were investigated. 7 paired corneas, one treated with palmitate-BSA and the mate cornea treated with BSA only as a control, for 5 days, were tested. The only significant respiration change found was in proton leak (Table 3, FIG. 38), which increased by 64% (p=0.045) compared to controls.

### Apoptosis and Necrosis:

Compared to controls, cells treated with ubiquinol had no change in cell necrosis (p=0.694) or apoptosis (p=0.517; Table 2, FIG. 36). In contrast, cells treated with palmitate-BSA had a 90% increase in necrosis (p=0.024) and 200% increase in apoptosis (p=0.028; Table 3, FIG. 38).

TABLE 2
Difference in mitochondrial metabolic parameters (n = 13 matched tissues) and % apoptotic and
% necrotic cells (n = 9 matched tissues) between ubiquinol treated and matched control tissues.

| | | | Mean or Median | |
|---|---|---|---|---|
| | | | Difference: Ubiquinol- | |
| | | | Control; | |
| | | | or Mean Ratio: | |
| Mitochondrial Metabolic | Mean (SD) or Median [IQR] | | Ubiquinol/Control | |
| Parameter | Ubiquinol | Control | (95% CI) | P-value* |
| Basal | 0.0202 (0.0087) | 0.0180 (0.0072) | Ratio: 1.12 (0.88, 1.43) | 0.324 |
| ATP Production | 0.0116 (0.0049) | 0.0117 (0.0062) | −0.0001 (−0.0033, 0.0032) | 0.962 |
| Proton Leak | 0.0091 (0.0049) | 0.0051 (0.0111) | Ratio: 1.80 (1.01, 3.21) | 0.047 |
| Maximal Respiration | 0.0998 (0.0799) | 0.0516 (0.0420) | Ratio: 1.93 (1.31, 2.85) | 0.003 |
| Spare Respiratory Capacity | 0.0781 (0.0734) | 0.0285 (0.0420) | Ratio: 2.74 (1.61, 4.66) | 0.001 |
| Non-mitochondrial Respiration | 0.00662 | 0.00832 | 0.000013^{#} (−0.00255, 0.00123) | 0.636 |
| | [0.00026- | [0.00025- | | |
| | 0.00790] | 0.00950] | | |
| Coupling Efficiency | 0.5303 (0.1141) | 0.5959 (0.1880) | −0.0655 (−0.1550, 0.0240) | 0.137 |
| % Necrotic | 0.0263 (0.0070) | 0.0257 (0.0076) | Ratio: 1.02 (0.91, 1.15) | 0.694 |
| % Apoptotic | 0.0040 (0.0047) | 0.0044 (0.0096) | Ratio: 0.90 (0.63, 1.29) | 0.517 |

*P-value from paired t-test for normally distributed differences and for difference with lognormal distribution (shown as mean ratio); and from Wilcoxon signed-rank test for differences that are not normally distributed (shown as ^{#}median difference).

TABLE 3
Difference in mitochondrial metabolic parameters (n = 7 matched tissues) and % apoptotic and
% necrotic cells (n = 8 matched tissues) between palmitate-BSA treated and matched control tissues.

| | | | Mean or Median | |
|---|---|---|---|---|
| | | | Difference: Palmitate- | |
| | | | Control; | |
| | | | or Mean Ratio: | |
| Mitochondrial Metabolic | Mean (SD) or Median [IQR] | | Palmitate/Control | |
| Parameter | Ubiquinol | Control | (95% CI) | P-value* |
| Basal | 0.0251 (0.0138) | 0.0207 (0.0017) | Ratio: 1.12 (0.74, 1.98) | 0.378 |
| ATP Production | 0.0155 (0.0090) | 0.0138 (0.0036) | 0.0017 (−0.0066, 0.0100) | 0.628 |
| Proton Leak | 0.0106 (0.0075) | 0.0064 (0.0034) | Ratio: 1.64 (1.02, 2.66) | 0.045 |
| Maximal Respiration | 0.1102 (0.0309) | 0.1023 (0.0321) | Ratio: 1.08 (0.72, 1.62) | 0.670 |
| Spare Respiratory Capacity | 0.0833 (0.0212) | 0.0808 (0.0323) | Ratio: 1.03 (0.66, 1.60) | 0.872 |
| Non-mitochondrial Respiration | 0.00809 | 0.00856 | −0.00075^{#} (−0.00928, 0.00644) | 0.688 |
| | [0.00412- | [0.00632- | | |
| | 0.01276] | 0.01132] | | |
| Coupling Efficiency | 0.5376 (0.1891) | 0.6521 (0.1442) | −0.1144 (−0.2602, 0.0313) | 0.103 |
| % Necrotic | 0.0628 (0.0532) | 0.0330 (0.0152) | Ratio: 1.90 (1.21, 3.23) | 0.024 |
| % Apoptotic | 0.00053 (0.00062) | 0.00018 (0.00022) | Ratio: 3.00 (1.18, 7.67) | 0.028 |

*P-value from paired t-test for normally distributed differences and for difference with lognormal distribution (shown as mean ratio); and from Wilcoxon signed-rank test for differences that are not normally distributed (shown as ^{#}median difference).

### Discussion

The study indicates that supplementation of hypothermic corneal storage media with 10 μM ubiquinol increases mitochondrial respiration in donor corneal endothelial tissue. Ubiquinol increased spare respiratory capacity and maximal respiration in CECs, and was not toxic as indicated by apoptosis and necrosis assay results that did not differ from controls. On the other hand, palmitate-BSA supplementation was toxic to donor CECs at the 100 μM dose tested and indicates the need for dose reduction in any future testing. Palmitate significantly increased both apoptosis and necrosis, but provided no mitochondrial enhancement. Additionally, bioenergetic plot profiles in the palmitate experimental controls (non-buffered assay media with BSA) were increased compared to ubiquinol controls (non-buffered assay media with ethanol), indicating that BSA may confer an enhancing effect and may have masked further negative effects of palmitate on CEC function. The findings for palmitate were the opposite of the expectation. Palmitate has been employed as an spare respiratory capacity enhancing agent in other systems (Pfleger et al., 2015). However, the data—in line with a recent study indicating that palmitate is toxic in mouse CECs (Bu et al., 2020) indicate instead that palmitate may instead be utilized as a positive disease control in future CEC studies. Overall, this study demonstrates that supplementing corneal storage media with ubiquinol may increase CEC mitochondrial function, and supports the need for further investigations into ubiquinol as an antioxidant with possible cytoprotective benefits for corneal endothelial cells.

Although the mechanisms of action for ubiquinol are well known—it is a component of the mitochondrial electron transport chain and ATP biosynthesis, and an effective fat-soluble antioxidant bound to cell and mitochondrial membranes that protects against reactive oxygen species mediated damage (Diaz-Casado et al., 2019; Ebadi et al., 2001; Hirst et al., 2016; Mellors et al., 1966) the precise mechanisms for its efficacy in donor tissue CECs require further investigation. Humans synthesize coenzyme Q10 and dietary ingestion generally is sufficient, making it unlikely that deficiency states are the reason for ubiquinol's efficacy in the experiments. The data, which indicate that supraphysiologic oxygen levels are present throughout the entirety of the conventional corneal storage period, suggest that oxygen mediated damage mechanisms may be contributing to relative cell dysfunction that is being rescued by ubiquinol. Recent findings from the Cornea Preservation Time Study demonstrated a decline in the 3-year DSAEK transplant survival rate with tissue preserved for >12 days compared to <11 days; thus, the duration of time that CECs spend in hypothermic storage has a significant clinical impact. It is hypothesize that ROS accumulation and oxidative damage may play an important role in donor CEC impairment related to presurgical hypothermic preservation. Elevated levels of ROS lead to macromolecule damage and cell death, and has been implicated in CEC dysfunction in vivo during Fuchs endothelial corneal dystrophy disease progression and postsurgically in an animal model (Benischke et al., 2017; Jurkunas et al., 2015; Jurkunas et al., 2010; Rahal et al., 2014; Wojcik et al., 2003; Zhao et al., 2016) Careful experimentation attuned to the presence of ROS in corneal storage conditions would be helpful in confirming this hypothesis, so that further studies regarding the effects of ubiquinol supplementation on donor corneal tissue performance and keratoplasty outcomes can be conducted.

Due to its long hydrocarbon side chain, ubiquinol is difficult to solubilize in biocompatible solvents. In this series, several attempts were made to bring this lipid-soluble molecule into aqueous solution. First, ubiquinol was attempted to be dissolved using polar organic solvents known to be biocompatible with CECs (Optisol-GS, water) based on the goal of achieving a high bioavailability for clinical applications. However, ubiquinol precipitated out of these solutions, even after heating. Next two organic polar aprotic solvents, DMSO and absolute ethanol, were tested. DMSO commonly is used as a solvent in cell biology and biochemistry, and both DMSO and ethanol can solubilize hydrocarbons. Despite its nonpolar moiety, ubiquinol precipitated in DMSO, also despite heating the solution. Ubiquinol was dissolved in absolute ethanol when heated to 37° C.; however, if this mixture was not poured immediately into the corneal storage media, the ubiquinol precipitated out of solution. Once dissolved in Optisol-GS storage media, ubiquinol appeared to remain in solution; however, the mechanism for its solubility in this solution remains unknown. Ubiquinol also precipitated out of solution in cell culture environments when attempting to perform additional assays in cell culture (data not shown). In addition to its lipophilicity, native ubiquinol is also unstable. Although not encountered in this series ubiquinol oxidizes in the presence of oxygen and light and turns yellow, indicating the formation of its oxidized form, ubiquinone. It is therefore necessary to improve the solubility and handleability of ubiquinol for future validations of its effects on oxidative stress related pathways.

There is an unclear significance related to mitochondrial proton leak demonstrated by the use of both supplements compared to controls; however, proton leak was not associated with reduced cell viability after ubiquinol supplementation. These findings do not raise significant concerns regarding ubiquinol toxicity at this dose presently. It was hypothesized that proton leak may be related to the concentration tested in this study.

In conclusion, testing in donor tissue at specified doses indicates ubiquinol may be a useful biocompatible additive to cornea storage media that increases CEC mitochondrial function in donor tissue, whereas palmitate-BSA reduces donor CEC viability. Ubiquinol, as an antioxidant with possible protective benefits for the corneal endothelium, may be studied and further developed for use in protecting donor CECs that are exposed to supraphysiologic concentrations of oxygen during hypothermic storage. Antioxidant supplementation of hypothermic corneal storage media may represent a viable strategy for improving the quality, availability, and surgical performance of donor corneal tissue used for keratoplasty.

### Example 14

The human corneal endothelium is made up of a single layer of hexagonal cells whose main function is to keep the cornea clear using ion pumping to counteract the passive leak of fluids into the stroma. Activity of these cells is energy dependent, requiring ATP, produced via aerobic mitochondrial metabolism under normoxic conditions. Overall, alterations in mitochondrial function may

impact the health of transplanted and native corneal tissue. In studies of Descemet stripping automated endothelial keratoplasty (DSAEK), mean endothelial cell density (ECD) drops by approximately 25% to 35% 6 months after surgery, which
represents a substantial decline compared to full thickness penetrating keratoplasty (PK) at the same time point (Terry et al., 2008; Price et al., 2008; Li et al., 2008). Data from the major Descemet membrane endothelial keratoplasty (DMEK)
surgical outcomes studies mirror the same trend, with mean 6-month postoperative ECD loss ranging from 27% to 37% (Rodriguez-Calvo; de-Mora et al., 2015; Feng et al., 2014; Hamzaoglu et al., 2015). Traditionally, this has been attributed to surgical technique and surgeon experience, but data from Bhogal et al. (2016) demonstrate a
14.5% ECD loss due to DMEK tissue preparation alone.

### Introduction

Experiments were conducted to determine if adding the antioxidant coenzyme Q10 (coQ10 or ubiquinol) to donor cornea storage media enhances the metabolic function of corneal endothelial cells (CECs) and/or decreases overall cell death in storage. The hypothesis was that a proportion of endothelial cells are predisposed to cell death before graft preparation and surgery so that adding antioxidant coenzyme Q10, e.g., to Optisol GS corneal storage medium, bolsters CEC function, health, and viability in storage.

### Materials and Methods

Tissue and Storage: Corneas used in this study were suitable for endothelial transplant, had consent for use in research, and were assayed within 14 days of preservation. All tissue experiments conformed to Declaration of Helsinki and UIowa IRB. Paired corneas were treated with mitochondrial enhancing compounds added to Optisol GS (Bausch & Lomb): 1 mM ascorbate-2-phosphate (24 hours), 10 mM palmitate-BSA (5 days), or 10 μM coenzyme Q10 (5 days). Treatments were only added to one cornea, while the cornea mates were treated with diluent only as the controls.

Mitochondrial Respiration Assay: 3 mm punches of central and peripheral endothelium-Descemet membrane complex (EDM) were secured to the bottom of cell culture microplate wells or CECs were grown directly onto microplate. Mitochondrial respiration was assayed on a Seahorse XFe24 extracellular flux analyzer (Seahorse Bioscience) following the manufacturer suggested protocols and Greiner et al. (2015) and Aldrich et al. (2017).

Apoptosis/Necrosis Assay: Remaining tissue was mounted onto slides and labeled with antibodies (anti-annexin IV, a marker for cell apoptosis) and counterstained with a nuclear stain (DAPI). Nuclei were counted for each punch to normalize respirometry data and immunohistochemistry densitometry using an Olympus IX81 inverted microscope with a UV filter.

### Results

Mitochondrial respiration results. (A) Seahorse XFe24 extracellular flux analysis output metrics diagram. Oxygen consumption rate per cell (OCR) of CECs treated with 1 mM ascorbate-2-phosphate (red) compared to controls (blue) (FIG. 38). OCR of CECs treated with 10 mM palmitate-BSA (red) compared to controls (blue) (FIG. 39). OCR of CECs from 14 paired corneas, one cornea was treated with 10 μM coenzyme Q10 (red) and the other cornea treated with diluent only as a control (blue) (FIG. 37). Dashed lines represent injections of oligomycin (O), carbonyl cyanide-p-trifluoromethoxy-phenylhydrazone (F), and antimycin A/rotenone (A/R).

Apoptosis/necrosis assay results. CHC necrosis did not change (P=0.85), but apoptosis was 29% lower in cells treated with coenzyme Q10 in storage (P=0.09) (FIG. 37). This indicates that not only did enzyme coQ10 boost the mitochondrial function of the endothelial cells, but may also prevent cells from dying in storage.

### Conclusions

Enzyme coQ10 is a safe additive to cornea storage media that enhances the function of the corneal endothelial cell mitochondria and decreases their overall death. On the other hand, palmitate-BSA proved to be toxic to corneal endothelial cells, increasing the amount of cell death in storage and ascorbate-2-phosphate did not appear to alter storage conditions at all. Thus, coenzyme Q10 is a supplement that may enhance transplant tissue and reduce graft failure overall in the future.

### REFERENCES

Aldrich et al., Invest. Opthalmol. Vis. Sci., 58:2130 (2017).
Awad et al., J. Thromb. Haemost., 11:1716 (2013).
Benischke et al., Sci. Rep., 7:6656 (2017).
Bhogal et al., Br. J. Ophthalmol., 12:e0184824 (2016).
Bu et al., American Journal of Pathology, (2020).
Diaz-Casado et al., Nutrients., 11 (2019).
Ebadi et al., Biol. Signals. Recept., 10:224 (2001).
Erdinest et al., Investig. Ophthalmol. Vis. Sci., 53:4396 (2012).
Esquenazi et al., Investig. Ophthalmol. Vis. Sci., 46:3121 (2005).
Feng et al., J. Cataract Refract. Surg., 40:1116 (2014).
Flynn et al., Free Radic Biol Med. 50(7):866 (2011).
Fuller et al., J. Cosmet. Dermatol., 5:30 (2006).
Goldstein et al., J. Ocul. Pharmacol. Ther., (2018).
Greiner et al., Invest. Ophthalmol. Vis. Sci., 56:2803 (2015).
Hajmousa et al., Biomaterials, 119:43 (2017).
Hamzaoglu et al., Ophthalmology, 122:2193 (2015)
Hirst and Roessler, Biochim. Biophys. Acta., 1857:872 (2016).
Huang et al., Invest Ophthalmol. Vis. Sci., 56:6483 (2015).
Jassem et al., J. Bioenerg. Biomembr., 38:49 (2006).
Jurkunas et al., Am. J. Pathol., 177:2278 (2010).
Jurkunas, Cornea., 37 Suppl 1:S50-S54 (2018).
Labate et al., Investigative ophthalmology & visual science, 58:179 (2017).
Lass et al., JAMA Ophthalmol., 135:1394 (2017).
Lass et al., JAMA Ophthalmol., doi: 10.1001/jamaophthalmol.2018.5669 (2018).
Li et al., Ophthalmology, 119:90 (2012).
Liu et al., J. Ocular Pharmacol., 10:587 (1994)).
Mellors and Tappel, J. Biol. Chem., 241:4353 (1966).
Miyai et al., Am. J. Pathol., (2019).
Onur et al., BioFactors, 40:346 (2014)).
Pfleger et al., Cell Death Dis., 6:e1835 (2015).
Price et al., Ophthalmology, 115:857 (2008).
Rahal et al., Biomed. Res. Int., 2014:761264 (2014).
Reddy et al., Br. J. Ophthalmol., 73:803 (1989).
Rodriguez-Calvo-de-Mora et al., Ophthalmology, 122:464 (2015).
Rosenwasser et al., JAMA Ophthalmol., 135:1401 (2017).
Saarinen-Savolainen et al., Pharm. Res., 15:1275 (1998).
Sahoo et al., BioMed. Res. Internat., 263604 (2014)).
Saokham et al., Molecules, 23(5) (2018).
Schipper et al., J. Cardiovasc. Transl. Res., 9:176 (2016).
Schneider et al., Nat. Methods, 9:671 (2012).
Skeie et al., PLoS One., 13(3):e0192287 (2018).
Tachibana et al., Jap. J. Ophthamol., 46:377 (2002).
Terry et al., Ophthalmology, 125:1700 (2018).
Terry et al., Cornea, 27:1131 (2008).
Wojcik et al., Int. J. Mol. Sci., 14:19294 (2013).
Zhao et al., BMC Ophthalmol., 16:16 (2016).
Zhao et al., RSC Advances, 7:28865 (2017).

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A corneal preservation composition comprising an effective amount of an anti-oxidant comprising one or more of ubiquinol, MitoQ, vitamin E, vitamin C, ascorbate-2-phosphate, idebenone, pyrroloquinoline quinone (PQQ), N-Acetyl-L-cysteine (NAC), palmitate, reduced glutathione, or a C14-C18 saturated fatty acid.

2-3. (canceled)

4. The composition of claim 1 wherein the fatty acid comprises palmitic acid, BSA-palmitate, docosahexaenoic acid (DHA), eicosapentaenoic acid (EPA) and/or alpha-linolenic acid.

5. The composition of claim 1 further comprising an amount of chondroitin sulfate or one or more omega 3 fatty acids.

6. (canceled)

7. The composition of claim 1 further comprising one or more carriers.

8. The composition of claim 7 wherein the carrier comprises cyclodextrin, polyethylene glycol (PEG), PEG dodecyl ether (Brij L4®), PEG hexadecyl ether (Brij 58®), lipid-based solubilizers like Labrafil® and Labrafac®, pluronics, e.g. Pluronic F68 (Poloxamer 188), polysorbate 80 and 20 or lipid nanoparticles.

9. The composition of claim 8 wherein the carrier comprises gamma-cyclodextrin.

10. The composition of claim 9 wherein the anti-oxidant comprises solubilized ubiquinol.

11-12. (canceled)

13. The composition of claim 1 further comprising a full thickness cornea, a partial thickness cornea or corneal endothelium.

14-17. (canceled)

18. The composition of claim 7 wherein the anti-oxidant and the carrier form complexes.

19. The composition of claim 18 wherein the complexes are about 200 to about 400 nm, about 100 to about 300 nm, about 300 to about 500 nm in diameter, or up to about 1000 nm in diameter.

20-21. (canceled)

22. The composition of claim 18 wherein the concentration of the complexes comprises about 0.1 μM to about 150 μM.

23-25. (canceled)

26. A method of making complexes of one or more anti-oxidants comprising ubiquinol, idebenone, vitamin A, vitamin C, PQQ, NAC, ascorbate-2-phosphate, reduced glutathione, vitamin E, or a C14-C18 saturated fatty acid, and a carrier, comprising: combining an amount of the one or more anti-oxidants and an amount of a carrier under low light and low oxygen conditions so as to form complexes of about 100 to about 500 nm in diameter or up to about 1000 nm in diameter.

27-28. (canceled)

29. A method of preserving a cornea, corneal tissue or corneal endothelium of a mammal, comprising:
providing a cornea, corneal tissue or corneal endothelium of a mammal; and combining the cornea, corneal tissue or corneal endothelium and the composition of claim 1.

30. The method of claim 29 wherein the cornea, corneal tissue or corneal endothelium is stored for up to 14 to 21 days at 2-40° C. prior to transplant.

31-34. (canceled)

35. A method of treating corneal endothelium, corneal epithelium, corneal keratocytes, corneal stroma, corneal nerves, conjunctival epithelium, conjunctival stroma, Tenon's capsule, trabecular meshwork, corneoscleral angle, lens epithelium, or lens tissue in a mammal, comprising administering to a mammal in need thereof an effective amount of the composition of claim 1.

36. (canceled)

37. The method of claim 35 wherein the mammal has diabetes or prediabetes, has an ocular disease or is a candidate for surgery.

38-39. (canceled)

40. The method of claim 37 wherein the ocular surgery includes cataract surgery, keratoplasty, removal of corneal tissue or lesions, ocular surface surgery including but not limited to pterygium surgery and lesion biopsies, vitreoretinal surgery, or glaucoma surgery.

41. (canceled)

42. The method of claim 35 wherein the composition is administered during, and/or after ocular surgery.

43. The method of claim 35 wherein the mammal has Fuchs endothelial corneal dystrophy.

44. An intraocular device for drug delivery comprising the composition of claim 1.

45. (canceled)
